(19) United States
(12) Patent Application Publication
(10) Pub. No.: US 2008/0300280 A1
(43) Pub. Date: Dec. 4, 2008

(54) Cyanobenzene Compounds for Combating Animal Pests

(76) Inventors: Matthias Pohlman, Heidelberg (DE); Wolfgang von Deyn, Neustadt (DE); Thomas Schmidt, Neustadt (DE); Florian Kaiser, Mannheim (DE); Douglas D. Anspaugh, Apex, NC (US); Deborah L. Culbertson, Fuquay Varina, NC (US); Henry Van Tuyl Cotter, Raleigh, NC (US)

Correspondence Address: BRINKS, HOFER, GILSON & LIONE, 2801 SLATER ROAD, SUITE 120, MORRISVILLE, NC, 27560, US

(21) Appl. No.: 12/094,942

(22) PCT Filed: Nov. 24, 2006
(86) PCT No.: PCT/EP2006/068880

§ 371 Date: May 23, 2008

Related U.S. Application Data
(60) Provisional application No. 60/739,642, filed on Nov. 25, 2005.

(51) Int. Cl. A01N 37/34; C07C 311/51; C07C 311/48; A01P 7/04; A01N 43/40; C07C 311/39

(52) U.S. Cl. 514/327; 558/413; 514/603

(57) ABSTRACT

The present invention relates to a method of combating animal pests which comprises contacting the animal pests, their habit, breeding ground, food supply, plant, seed, soil, area, material or environment in which the animal pests are growing or may grow, or the materials, plants, seeds, soils, surfaces or spaces to be protected from animal attack or infestation with a pesticidally effective amount of at least one cyanobenzene compound of the formula (I) and/or at least one agriculturally acceptable salt thereof: where m is 0, 1 or 2; is a radical of the formulae N═CR⁵R⁶, N═SR⁷R⁸, NR¹⁰—C(═X)—R⁹, where X is O, S A or NR¹¹, or A is a N-bound 5-, 6- or 7-membered heterocycle, which is ethylenically unsaturated or aromatic, and which additionally may contain 1, 2, or 3 further heteroatoms or heteroatom groups, selected from O, S, SO, SO₂, N, and NR¹², and/or 1, 2 or 3 carbonyl groups as ring members and which may carry 1, 2, 3 or 4 radicals R¹³, where the radicals R¹ to R¹³ are as defined in the claims or the description. The invention further relates to specific compounds I and to agricultural compositions comprising them.

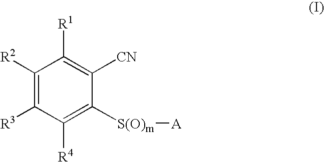

The present invention relates to a method of combating animal pests by using cyano-benzene compounds and/or agriculturally acceptable salts thereof. The invention also relates to certain cyanobenzene compounds and/or their agriculturally acceptable salts and also to compositions comprising them.

Animal pests destroy growing and harvested crops and attack wooden dwelling and commercial structures, causing large economic loss to the food supply and to property. While a large number of pesticidal agents are known, due to the ability of target pests to develop resistance to said agents, there is an ongoing need for new agents for combating animal pests. In particular, animal pests such as insects and acaridae are difficult to be effectively controlled.

EP 33984 discloses 2-cyanobenzene sulfonamides having aphicidal activity. Their activity, however, is not satisfactory. Similar compounds are described in WO 2005/035486.

EP-A-0303863 describes benzenesulfonyl-2-imidazolin-5-one compounds which are useful as herbicides. An activity against animal pests is not mentioned.

EP-A-0107624 describes a method for producing sulfonylurea compounds by reacting a sulfonylamidocarbonic diester with an amino-substituted pyrimidine or triazine. The sulfonylurea compounds are said to have a herbicidal activity.

D.-H. Kweon et al. describe in J. Heterocyclic Chem. 39, 2002, 203 the synthesis of certain pyridazinones. The compounds are supposed to be useful agrochemicals. An activity against animal pests is not specifically described.

N. Masuda et al. describe in Bioorganic and Medicinal Chemistry 13, 2005, 9492-cyano-N-(4,5-dialkylthiazol-2-yl)-benzenesulfonamides. These compounds are used as intermediates for the synthesis of 2-cyano-N-(3-methyl-4,5-dialkylthiazol-2(3H)-ylidene)-benzenesulfonamides which are said to have a potent anti-HIV-1 activity.

The object of the present invention is to provide compounds having a good pesticidal activity, especially against difficult to control insects and acaridae.

It has been shown that this object is achieved by a method of combating animal pests which comprises contacting the animal pests, their habit, breeding ground, food supply, plant, seed, soil, area, material or environment in which the animal pests are growing or may grow, or the materials, plants, seeds, soils, surfaces or spaces to be protected from animal attack or infestation with a pesticidally effective amount of at least one cyanobenzene compound of the formula I and/or at least one agriculturally acceptable salt thereof:

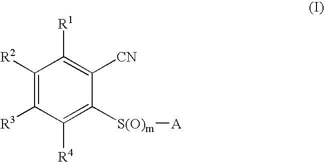

where
m is 0, 1 or 2;
A is a radical of the formulae
   N═CR⁵R⁶, N═SR⁷R⁸, NR¹⁰—C(═X)—R⁹,
   where X is O, S or NR¹¹,
   or A is a N-bound 5-, 6- or 7-membered heterocycle, which is ethylenically unsaturated or aromatic, and which additionally may contain 1, 2, or 3 further heteroatoms or heteroatom groups, selected from O, S, SO, SO₂, N, and NR¹², and/or 1, 2 or 3 carbonyl groups as ring members and which may carry 1, 2, 3 or 4 radicals R¹³,
R¹ is hydrogen, nitro, cyano, azido, amino, halogen, sulfenylamino, sulfinylamino, sulfonylamino, C(═O)R¹⁴, C₁-C₆-alkyl, C₂-C₆-alkenyl, C₂-C₆-alkynyl, C₃-C₈-cycloalkyl, C₁-C₆-alkoxy, (C₁-C₆-alkyl)amino, di(C₁-C₆-alkyl)amino, C₁-C₆-alkylthio, C₁-C₆-alkylsulfinyl or C₁-C₆-alkylsulfonyl, wherein the ten last-mentioned radicals may be unsubstituted, partially or fully halogenated and/or may carry 1, 2 or 3 radicals, selected from the group consisting of cyano, nitro, amino, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, (C₁-C₄-alkoxy)carbonyl, (C₁-C₄-alkyl)amino, di(C₁-C₄-alkyl)amino, C₃-C₈-cycloalkyl and phenyl, it being possible for phenyl to be unsubstituted, partially or fully halogenated and/or to carry 1, 2 or 3 substituents, independently of one another selected from the group consisting of C₁-C₄-alkyl, C₁-C₄-haloalkyl, C₁-C₄-alkoxy and C₁-C₄-haloalkoxy;
R², R³ and R⁴ are independently of one another selected from the group consisting of hydrogen, halogen, cyano, azido, nitro, C₁-C₆-alkyl, C₃-C₈-cycloalkyl, C₁-C₄-haloalkyl, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, C₂-C₆-alkenyl, C₂-C₆-alkynyl, amino, (C₁-C₄-alkyl)amino, di(C₁-C₄-alkyl)amino, sulfonylamino, sulfinylamino, sulfenylamino and C(═O)—R¹⁵;
R⁵ is H, OR^{5a}, NR^{5b}R^{5c}, aryl, aryl-C₁-C₄-alkyl, heteroaryl, heteroaryl-C₁-C₄-alkyl, heterocyclyl, heterocyclyl-C₁-C₄-alkyl, C₁-C₁₀-alkyl, C₃-C₁₀-cycloalkyl, C₁-C₁₀-alkylthio, C₁-C₁₀-alkylsulfonyl, C₁-C₁₀-alkylsulfinyl, wherein the five last-mentioned radicals may be unsubstituted, partially or fully halogenated and/or may carry 1, 2 or 3 radicals, independently of one another each selected from the group consisting of cyano, nitro, amino, C₁-C₁₀-alkoxy, C₁-C₁₀-alkylthio, C₁-C₁₀-alkylsulfinyl, C₁-C₁₀-alkylsulfonyl, C₁-C₁₀-haloalkoxy, C₁-C₁₀-haloalkylthio, C₁-C₁₀-alkoxycarbonyl, (C₁-C₁₀-alkyl)amino, di-(C₁-C₁₀-alkyl)amino, C₃-C₁₀-cycloalkyl and phenyl, it being possible for phenyl to be unsubstituted, partially or fully halogenated and/or to carry 1, 2 or 3 substituents, independently of one another selected from the group consisting of C₁-C₄-alkyl, C₁-C₄-haloalkyl, C₁-C₄-alkoxy and C₁-C₄-haloalkoxy;
   wherein
   R^{5a} is C₁-C₁₀-alkyl, C₃-C₁₀-cycloalkyl, C₂-C₁₀-alkenyl, C₂-C₁₀-alkynyl, aryl, aryl-C₁-C₄-alkyl, heteroaryl or heteroaryl-C₁-C₄-alkyl, heterocyclyl or heterocyclyl-C₁-C₄-alkyl;
   R^{5b}, R^{5c}, independently from each other, are selected from hydrogen, C₁-C₁₀-alkyl, C₃-C₁₀-cycloalkyl, C₂-C₁₀-alkenyl, C₂-C₁₀-alkynyl, aryl, aryl-C₁-C₄-alkyl, heteroaryl and heteroaryl-C₁-C₄-alkyl, heterocyclyl or heterocyclyl-C₁-C₄-alkyl;
   wherein C₁-C₁₀-alkyl, C₃-C₁₀-cycloalkyl, C₂-C₁₀-alkenyl and C₂-C₁₀-alkynyl in R^{5a}, R^{5b} and R^{5c} may be unsubstituted, partially or fully halogenated and/or may carry 1, 2 or 3 radicals, selected from the group consisting of cyano, nitro, amino, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, (C₁-C₄-alkoxy)carbonyl, (C₁-C₄-alkyl)amino, di(C₁-C₄-alkyl)amino and C₃-C₈-cycloalkyl;
   wherein the heteroaryl moiety in heteroaryl and heteroaryl-C₁-C₄-alkyl of R⁵, R^{5a}, R^{5b} and R^{5c} is 5- or 6 membered and contains 1, 2, 3 or 4 heteroatoms and/or heteroatom groups, selected from O, S, SO, SO₂, N or NR^{a}, as ring members, R^{a} being hydrogen or C₁-C₆-alkyl;
   wherein the heterocyclyl moiety in heterocyclyl and heterocyclyl-C₁-C₄-alkyl of R⁵, R^{5a}, R^{5b} and R^{5c} is 3- to 7 membered, is saturated or partly unsaturated and contains 1, 2, 3 or 4 heteroatoms and/or heteroatom groups, selected from O, S, SO, SO₂, N or NR^{b}, as ring members, R^{b} being hydrogen or C₁-C₆-alkyl, and additionally may contain 1, 2 or 3 CO groups as ring members;
   and wherein the carbon atoms of aryl, hetaryl, and heterocyclyl moieties in R⁵, R^{5a}, R^{5b} and R^{5c} may be unsubstituted or may carry 1, 2 or 3 substituents, independently of one another selected from the group consisting of halogen, C₁-C₄-alkyl, C₁-C₄-haloalkyl, C₁-C₄-alkoxy and C₁-C₄-haloalkoxy;
R⁶ independently has one of the meanings given for R⁵;
R⁷, R⁸, independently from each other, are selected from aryl, aryl-C₁-C₄-alkyl, heteroaryl, heteroaryl-C₁-C₄-alkyl, heterocyclyl, heterocyclyl-C₁-C₄-alkyl, C₁-C₁₀-alkyl, C₃-C₁₀-cycloalkyl, C₂-C₁₀-alkenyl and C₂-C₁₀-alkynyl, wherein the four last-mentioned radicals may be unsubstituted, partially or fully halogenated and/or may carry 1, 2 or 3 radicals, independently of one another each selected from the group consisting of cyano, nitro, amino, C₁-C₁₀-alkoxy, C₁-C₁₀-alkylthio, C₁-C₁₀-alkylsulfinyl, C₁-C₁₀-alkylsulfonyl, C₁-C₁₀-haloalkoxy, C₁-C₁₀-haloalkylthio, C₁-C₁₀-alkoxycarbonyl, (C₁-C₁₀-alkyl)amino, di-(C₁-C₁₀-alkyl)amino, C₃-C₁₀-cycloalkyl and phenyl, it being possible for phenyl to be unsubstituted, partially or fully halogenated and/or to carry 1, 2 or 3 substituents, independently of one another selected from the group consisting of C₁-C₄-alkyl, C₁-C₄-haloalkyl, C₁-C₄-alkoxy and C₁-C₄-haloalkoxy; or
R⁷ and R⁸ together with the sulfur atom they are bound to form a saturated or ethylenically unsaturated 5- to 10-membered ring, optionally substituted by 1, 2, 3 or 4 radicals selected from C₁-C₅-alkyl and halogen, wherein the ring may contain, in addition to the sulfur atom, 1, 2 or 3 heteroatoms and/or heteroatom-containing groups as ring members selected from the group consisting of nitrogen, oxygen, sulfur, CO, SO, SO₂ and N—R¹⁷;
   wherein the heteroaryl moiety in heteroaryl and heteroaryl-C₁-C₄-alkyl of R⁷ and R⁸ is 5- or 6 membered and contains 1, 2, 3 or 4 heteroatoms and/or heteroatom groups, selected from O, S, SO, SO₂, N or NR^{c}, as ring members, R^{c} being hydrogen or C₁-C₆-alkyl,
   wherein the heterocyclyl moiety in heterocyclyl and heterocyclyl-C₁-C₄-alkyl of R⁷ and R⁸ is 3- to 7-membered, is saturated or partly unsaturated and contains 1, 2, 3 or 4 heteroatoms and/or heteroatom groups, selected from O, S, SO, SO₂, N or NR^{d}, as ring members, R^{d} being hydrogen or C₁-C₆-alkyl, and additionally may contain 1, 2 or 3 CO groups as ring members,
   and wherein the carbon atoms of aryl, hetaryl, and heterocyclyl in R⁷ and R⁸ may be unsubstituted or may carry 1, 2 or 3 substituents, independently of one another selected from the group consisting of halogen, C₁-C₄-alkyl, C₁-C₄-haloalkyl, C₁-C₄-alkoxy and C₁-C₄-haloalkoxy;
R⁹ is selected from the group consisting of hydrogen, OR^{9a}, NR^{9b}R^{9c}, aryl, aryl-C₁-C₄-alkyl, heteroaryl, heteroaryl-C₁-C₄-alkyl, heterocyclyl, heterocyclyl-C₁-C₄-alkyl, C₁-C₁₀-alkyl, C₂-C₁₀-alkenyl, C₂-C₁₀-alkynyl and C₃-C₁₀-cycloalkyl, wherein the four last-mentioned radicals may be unsubstituted, partially or fully halogenated and/or may carry 1, 2 or 3 radicals, independently of one another each selected from the group consisting of cyano, nitro, amino, C₁-C₁₀-alkoxy, C₁-C₁₀-alkylthio, C₁-C₁₀-alkylsulfinyl, C₁-C₁₀-alkylsulfonyl, C₁-C₁₀-haloalkoxy, C₁-C₁₀-haloalkylthio, C₁-C₁₀-alkoxycarbonyl, (C₁-C₁₀-alkyl)amino, di-(C₁-C₁₀-alkyl)amino, C₃-C₁₀-cycloalkyl and phenyl, it being possible for phenyl to be unsubstituted, partially or fully halogenated and/or to carry 1, 2 or 3 substituents, independently of one another selected from the group consisting of C₁-C₄-alkyl, C₁-C₄-haloalkyl, C₁-C₄-alkoxy and C₁-C₄-haloalkoxy,
   and wherein
   R^{9a} is C₁-C₁₀-alkyl, C₃-C₁₀-cycloalkyl, C₂-C₁₀-alkenyl, C₂-C₁₀-alkynyl, aryl, aryl-C₁-C₄-alkyl, heteroaryl or heteroaryl-C₁-C₄-alkyl, heterocyclyl or heterocyclyl-C₁-C₄-alkyl,
   R^{9b}, R^{9c}, independently from each other, are selected from hydrogen, C₁-C₁₀-alkyl, C₃-C₁₀-cycloalkyl, C₂-C₁₀-alkenyl, C₂-C₁₀-alkynyl, aryl, aryl-C₁-C₄-alkyl, heteroaryl and heteroaryl-C₁-C₄-alkyl, heterocyclyl or heterocyclyl-C₁-C₄-alkyl,
      wherein C₁-C₁₀-alkyl, C₃-C₁₀-cycloalkyl, C₂-C₁₀-alkenyl and C₂-C₁₀-alkynyl in R^{9a}, R^{9b} and R^{9c} may be unsubstituted, partially or fully halogenated and/or may carry 1, 2 or 3 radicals, selected from the group consisting of cyano, nitro, amino, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, (C₁-C₄-alkoxy)carbonyl, (C₁-C₄-alkyl)amino, di(C₁-C₄-alkyl)amino and C₃-C₈-cycloalkyl,
      wherein the heteroaryl moiety in heteroaryl and heteroaryl-C₁-C₄-alkyl of R⁹, R^{9a}, R^{9b} and R^{9c} is 5- or 6-membered and contains 1, 2, 3 or 4 heteroatoms and/or heteroatom groups, selected from O, S, SO, SO₂, N or NR^{e}, as ring members, R^{e} being hydrogen or C₁-C₆-alkyl,
      wherein the heterocyclyl moiety in heterocyclyl and heterocyclyl-C₁-C₄-alkyl of R⁹, R^{9a}, R^{9b} and R^{9c} is 3- to 7-membered, is saturated or partly unsaturated and contains 1, 2, 3 or 4 heteroatoms and/or heteroatom groups, selected from O, S, SO, SO₂, N or NR^{f}, as ring members, R^{f} being hydrogen or C₁-C₆-alkyl, and additionally may contain 1, 2 or 3 CO groups as ring members,
      and wherein the carbon atoms of aryl, hetaryl and heterocyclyl moieties in R⁹, R^{9a}, R^{9b} and R^{9c} may be unsubstituted or may carry 1, 2 or 3 substituents, independently of one another selected from the group consisting of halogen, C₁-C₄-alkyl, C₁-C₄-haloalkyl, C₁-C₄-alkoxy and C₁-C₄-haloalkoxy;
R¹⁰ is selected from the group consisting of hydrogen, C(═O)—R¹⁶, C₁-C₁₀-alkyl, C₂-C₁₀-alkenyl, C₂-C₁₀-alkynyl, C₁-C₁₀-alkoxy and C₃-C₁₀-cycloalkyl, wherein the five last-mentioned radicals may be unsubstituted, partially or fully halogenated and/or may carry 1, 2 or 3 radicals, independently of one another each selected from the group consisting of cyano, nitro, amino, C₁-C₁₀-alkoxy, C₁-C₁₀-alkylthio, C₁-C₁₀-alkylsulfinyl, C₁-C₁₀-alkylsulfonyl, C₁-C₁₀-haloalkoxy, C₁-C₁₀-haloalkylthio, C₁-C₁₀-alkoxycarbonyl, (C₁-C₁₀-alkyl)amino, di-(C₁-C₁₀-alkyl)amino, C₃-C₁₀-cycloalkyl and phenyl, it being possible for phenyl to be unsubstituted, partially or fully halogenated and/or to carry 1, 2 or 3 substituents, independently of one another selected from the group consisting of C₁-C₄-alkyl, C₁-C₄-haloalkyl, C₁-C₄-alkoxy and C₁-C₄-haloalkoxy; or
R⁹ and R¹⁰ together with the adjacent nitrogen and carbon atoms form a saturated or ethylenically unsaturated 5 to 10-membered ring, optionally substituted by 1, 2, 3 or 4 radicals selected from C₁-C₅-alkyl and halogen, wherein the ring may contain, in addition to the nitrogen and carbon ring members, 1, 2 or 3 heteroatoms and/or heteroatom groups as ring members selected from the group consisting of nitrogen, oxygen, sulfur, CO, SO, SO₂ and N—R¹⁷;
R¹¹ is C₁-C₆-alkyl or C₁-C₆-alkoxy;
or
R¹¹ and R⁹ together with the adjacent nitrogen and carbon atoms form an ethylenically unsaturated aromatic or non-aromatic 5 to 10-membered ring, optionally substituted by 1, 2 or 3 C₁-C₅-alkyl radicals, wherein the ring may contain, in addition to the nitrogen and carbon ring members, 1, 2 or 3 heteroatoms and/or heteroatom groups as ring members selected from the group consisting of nitrogen, oxygen, sulfur, SO, SO₂ and N—R¹⁷;
or
R¹¹ and R¹⁰ together with the adjacent nitrogen and carbon atoms form an ethylenically unsaturated aromatic or non-aromatic 5 to 10-membered ring, optionally substituted by 1, 2 or 3 C₁-C₅-alkyl radicals, wherein the ring may contain, in addition to the nitrogen and carbon ring members, 1, 2 or 3 heteroatoms and/or heteroatom groups as ring members selected from the group consisting of nitrogen, oxygen, sulfur, SO, SO₂ and N—R¹⁷;
R¹² is hydrogen, C₁-C₆-alkyl, C₁-C₄-alkoxy-C₁-C₄-alkyl or C₁-C₆-alkoxy;
each R¹³ independently is selected from halogen, cyano, nitro, sulfenylamino, sulfinylamino, sulfonylamino, aryl, aryl-C₁-C₄-alkyl, heteroaryl, heteroaryl-C₁-C₄-alkyl, heterocyclyl, heterocyclyl-C₁-C₄-alkyl, C₁-C₆-alkyl, C₃-C₈-cycloalkyl, C₁-C₆-alkoxy, C₁-C₆-alkoxycarbonyl, C₁-C₆-alkylcarbonyl, (C₁-C₆-alkyl)amino, di(C₁-C₆-alkyl)amino, C₁-C₆-alkylthio, C₁-C₆-alkylsulfinyl or C₁-C₆-alkylsulfonyl, wherein the ten last-mentioned radicals may be unsubstituted, partially or fully halogenated and/or may carry 1, 2 or 3 radicals, selected from the group consisting of cyano, nitro, amino, OH, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, C₁-C₄-alkylcarbonyloxy, (C₁-C₄-alkoxy)carbonyl, (C₁-C₄-alkyl)amino, di(C₁-C₄-alkyl)amino, C₃-C₈-cycloalkyl and phenyl, it being possible for phenyl to be unsubstituted, partially or fully halogenated and/or to carry 1, 2 or 3 substituents, independently of one another selected from the group consisting of C₁-C₄-alkyl, C₁-C₄-haloalkyl, C₁-C₄-alkoxy and C₁-C₄-haloalkoxy,
   wherein the heteroaryl moiety in heteroaryl and heteroaryl-C₁-C₄-alkyl of R¹³ is 5- or 6 membered and contains 1, 2, 3 or 4 heteroatoms and/or heteroatom groups, selected from O, S, SO, SO₂, N or NR^{g}, as ring members, R^{g} being hydrogen or C₁-C₆-alkyl,
   wherein the heterocyclyl moiety in heterocyclyl and heterocyclyl-C₁-C₄-alkyl of R¹³ is 3- to 7-membered and contains 1, 2, 3 or 4 heteroatoms and/or heteroatom groups, selected from O, S, SO, SO₂, N or NR^{h}, as ring members, R^{h} being hydrogen or C₁-C₆-alkyl, and additionally may contain 1, 2 or 3 CO groups as ring members,
   and wherein the carbon atoms of aryl, hetaryl, and heterocyclyl in R¹³ may be unsubstituted or may carry 1, 2 or 3 substituents, independently of one another selected from the group consisting of halogen, C₁-C₄-alkyl, C₁-C₄-haloalkyl, C₁-C₄-alkoxy and C₁-C₄-haloalkoxy;
R¹⁴ and R¹⁵ independently of one another are selected from the group consisting of hydrogen, hydroxy, C₁-C₆-alkoxy, amino, C₁-C₄-alkylamino, di(C₁-C₄-alkyl)amino, aryl, aryl-C₁-C₄-alkyl, C₁-C₄-alkyl, where the alkyl moiety in the two last-mentioned radicals and the aryl moiety in aryl and aryl-C₁-C₄-alkyl may be partially or fully halogenated,
   3- to 7-membered heteroaryl or heteroaryl-C₁-C₄-alkyl, wherein the heteroaryl ring contains as ring members 1, 2 or 3 heteroatoms and/or heteroatom groups, selected from the group consisting of nitrogen, oxygen, sulfur, SO, SO₂ and N—Rⁿ, wherein Rⁿ is hydrogen or C₁-C₄-alkyl,
   3- to 7-membered heterocyclyl or heterocyclyl-C₁-C₄-alkyl, wherein the heterocyclic ring is saturated or partly unsaturated and contains 1, 2 or 3 heteroatoms and/or heteroatom groups, selected from the group consisting of nitrogen, oxygen, sulfur, SO, SO₂ and N—R^{o}, wherein R^{o} is hydrogen or C₁-C₄-alkyl,
   and wherein the carbon atoms of the heterocyclic rings may by unsubstituted or substituted by 1 or 2 radicals selected from halogen or C₁-C₄-alkyl;
R¹⁶ is C₁-C₆-alkyl, aryl, aryl-C₁-C₄-alkyl,
   5- to 7-membered heteroaryl or heteroaryl-C₁-C₄-alkyl, wherein the heteroaryl ring contains as ring members 1, 2 or 3 heteroatoms and/or heteroatom groups, selected from the group consisting of nitrogen, oxygen, sulfur, SO, SO₂ and N—R^{k}, wherein R^{k} is hydrogen or C₁-C₄-alkyl,
   3- to 7-membered heterocyclyl or heterocyclyl-C₁-C₄-alkyl, wherein the heterocyclic ring is saturated or partly unsaturated and contains as ring members 1, 2 or 3 heteroatoms and/or heteroatom groups, selected from the group consisting of nitrogen, oxygen, sulfur, SO, SO₂ and N—R^{m}, wherein R^{m} is hydrogen or C₁-C₄-alkyl,
   and wherein the carbon atoms of the heterocyclic rings may be unsubstituted or substituted by 1 or 2 radicals selected from halogen or C₁-C₄-alkyl;
or
R¹⁶ and R⁹ together with the adjacent nitrogen and carbon atoms form a saturated or ethylenically unsaturated 5 to 10-membered ring, where the ring may be partially or fully halogenated and/or may be substituted by 1, 2 or 3 substituents selected from C₁-C₅-alkyl and C₁-C₅-haloalkyl, wherein the ring may contain, in addition to the nitrogen and carbon ring members, 1, 2 or 3 heteroatoms and/or heteroatom groups as ring members selected from the group consisting of nitrogen, oxygen, sulfur, SO, SO₂ and N—R¹⁷;
each R¹⁷ independently is hydrogen, heteroaryl, heteroaryl-C₁-C₄-alkyl or C₁-C₆-alkyl, where the alkyl moiety in the two last-mentioned radicals may be partially or fully halogenated;
   and/or the agriculturally acceptable salts thereof.

The compounds of the formula I and their agriculturally acceptable salts are highly active against animal pest, i.e. harmful arthropodes and nematodes, especially against difficult to control insects and acaridae.

Moreover, the present invention relates to the use of compounds I and/or their salts for combating animal pests.

The invention is also related to seed comprising a compound of the formula I or an agriculturally useful salt of I, as defined above, in an amount of from 0.1 g to 10 kg per 100 kg of seed.

The invention further relates to compounds of the formula (I) and agriculturally acceptable salts thereof,

except for compounds of the formula I, wherein R¹ is H, NO₂ or NH₂ if R² is H, R³ is H, Cl or CO₂CH₃, R⁴ is H and A is a radical N═CH—N(CH₃)₂;
except for compounds of the formula I, wherein R¹ is H if R² is H or Cl, R³ is H, R⁴ is H and A is an optionally substituted pyridazin-6-on-1-yl radical or an optionally substituted imidazolin-5-on-1-yl radical;
except for compounds of the formula I, wherein R¹ is H if R² is H, R³ is H, R⁴ is H and A is a radical N═C(O-ethyl)₂; and
except for compounds of the formula I, wherein A is a radical of the formula

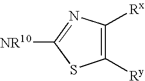

where R^{x} and R^{y} are, independently of each other, hydrogen or C₁-C₅-alkyl and R¹⁰ is H or C₁-C₁₀-alkyl;
in particular
except for compounds of the formula I, wherein R¹ is H, NO₂ or NH₂ if R² is H, R³ is H, Cl or CO₂CH₃, R⁴ is H and A is a radical N═CH—N(CH₃)₂;
except for compounds of the formula I, wherein R¹ and R² are both H;
except for compounds of the formula I, wherein R¹, R³ and R⁴ are H and R² is halogen; and
except for compounds of the formula I, wherein A is a radical of the formula

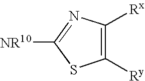

where R^{x} and R^{y} are, independently of each other, hydrogen or C₁-C₅-alkyl and R¹⁰ is H or C₁-C₁₀-alkyl.

Finally, the invention also relates to agricultural compositions comprising at least one cyanobenzene derivative of the formula I and/or at least one agriculturally useful salt of I and at least one inert liquid and/or solid agronomically acceptable carrier and, if desired, at least one surfactant. The compound I and/or its salt is of course comprised in such an amount that it has a pesticidal action.

The compounds of the general formula I may have one or more centers of chirality, in which case they are present as mixtures of enantiomers or diastereomers. The present invention provides both the pure enantiomes or diastereomers or mixtures thereof. The compounds of the general formula I may also exist in the form of different tautomers, e.g. if one of R^{5b} or R^{5c} is H or if X is NR¹¹ and R¹⁰ is H, but also in other cases. The invention comprises the single tautomers, if separable, as well as the tautomer mixtures. The compounds of formula I may further exist in the form of different stereoisomers, in particular E/Z-isomers, e.g. in case A is N═CR⁵R⁶, with R⁵ and R⁶ being different substituents, or in case A is N═SR⁷R⁸, with R⁷, R⁸ being different substituents. The invention comprises single stereoisomers as well as stereoisomer mixtures.

Salts of the compounds of the formula I are preferably agriculturally and veterinarily acceptable salts. They can be formed in a customary method, e.g. by reacting the compound with an acid of the anion in question if the compound of formula I has a basic functionality or by reacting an acidic compound of formula I with a suitable base.

Suitable agriculturally useful salts are especially the salts of those cations or the acid addition salts of those acids the cations and anions of which do not have any adverse effect on the action of the compounds according to the present invention. Suitable cations are in particular the ions of the alkali metals, preferably lithium, sodium and potassium, of the alkaline earth metals, preferably calcium, magnesium and barium, and of the transition metals, preferably manganese, copper, zinc and iron, and also ammonium (NH₄⁺) and substituted ammonium in which one to four of the hydrogen atoms are replaced by C₁-C₄-alkyl, C₁-C₄-hydroxyalkyl, C₁-C₄-alkoxy, C₁-C₄-alkoxy-C₁-C₄-alkyl, hydroxy-C₁-C₄-alkoxy-C₁-C₄-alkyl, phenyl or benzyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethylammonium, bis(2-hydroxyethyl)ammonium, benzyltrimethylammonium and benzyltriethylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogen sulfate, sulfate, dihydrogen phosphate, hydrogen phosphate, phosphate, nitrate, hydrogen carbonate, carbonate, hexafluorosilicate, hexafluorophosphate, benzoate, and the anions of C₁-C₄-alkanoic acids, preferably formate, acetate, propionate and butyrate. They can be formed by reacting the compounds of the formula I with an acid of the corresponding anion, preferably of hydrochloric acid, hydrobromic acid, sulfuric acid, phosphoric acid or nitric acid.

By the term “veterinarily acceptable salts” is meant salts of those cations or anions which are known and accepted in the art for the formation of salts for veterinary use. Suitable acid addition salts, e.g. formed by compounds of formula I containing a basic nitrogen atom, e.g. an amino group, include salts with inorganic acids, for example hydrochlorides, sulphates, phosphates, and nitrates and salts of organic acids for example acetic acid, maleic acid, dimaleic acid, fumaric acid, difumaric acid, methane sulfenic acid, methane sulfonic acid, and succinic acid.

The organic moieties mentioned in the above definitions of the variables are—like the term halogen—collective terms for individual listings of the individual group members. The prefix Cₙ-Cₘ indicates in each case the possible number of carbon atoms in the group.

“Halogen” will be taken to mean fluoro, chloro, bromo and iodo.

The term “partially or fully halogenated” will be taken to mean that 1 or more, e.g. 1, 2, 3, 4 or 5 or all of the hydrogen atoms of a given radical have been replaced by a halogen atom, in particular by fluorine or chlorine.

The term “Cₙ-Cₘ-alkyl” as used herein (and also in Cₙ-Cₘ-alkylamino, di-Cₙ-Cₘ-alkylamino, Cₙ-Cₘ-alkylaminocarbonyl, di-(Cₙ-Cₘ-alkylamino)carbonyl, Cₙ-Cₘ-alkylthio (synonymous with Cₙ-Cₘ-alkylsulfenyl), Cₙ-Cₘ-alkylsulfinyl and Cₙ-Cₘ-alkylsulfonyl) refers to a branched or unbranched saturated hydrocarbon group having n to m, e.g. 1 to 10 carbon atoms, preferably 1 to 6 carbon atoms, for example methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, heptyl, octyl, 2-ethylhexyl, nonyl and decyl and their isomers. C₁-C₄-alkyl means for example methyl, ethyl, propyl, 1-methylethyl, butyl, 1-methylpropyl, 2-methylpropyl or 1,1-dimethylethyl. C₁-C₅-alkyl additionally means pentyl, 1-methylbutyl, 2-methylbutyl, 3-methylbutyl, 1,1-dimethylpropyl, 2,2-dimethylpropyl, 1-ethylpropyl and constitutional isomers thereof. C₁-C₆-alkyl additionally means hexyl and constitutional isomers thereof. C₁-C₂-alkyl means methyl and ethyl.

The term “Cₙ-Cₘ-haloalkyl” as used herein refers to a straight-chain or branched alkyl group having n to m carbon atoms, e.g. 1 to 10 in particular 1 to 6 carbon atoms (as mentioned above), where some or all of the hydrogen atoms in these groups may be replaced by halogen atoms as mentioned above, for example C₁-C₄-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and the like. The term C₁-C₁₀-haloalkyl in particular comprises C₁-C₂-fluoroalkyl, which is synonym with methyl or ethyl, wherein 1, 2, 3, 4 or 5 hydrogen atoms are substituted by fluorine atoms, such as fluoromethyl, difluoromethyl, trifluoromethyl, 1-fluoroethyl, 2-fluoroethyl, 2,2-difluoroethyl, 2,2,2-trifluoroethyl and pentafluoromethyl.

Similarly, “Cₙ-Cₘ-alkoxy” and “Cₙ-Cₘ-alkylthio” (═Cₙ-Cₘ-alkylsulfenyl) (and also Cₙ-Cₘ-alkylsulfinyl or Cₙ-Cₘ-alkylsulfonyl) refer to straight-chain or branched alkyl groups having n to m carbon atoms, e.g. 1 to 10, in particular 1 to 6 or 1 to 4 carbon atoms (as mentioned above) bonded through oxygen or sulfur linkages (or SO (sulfinyl) or S(O)₂ linkages (sulfonyl)), respectively, at any bond in the alkyl group. Examples include C₁-C₄-alkoxy such as methoxy, ethoxy, propoxy, isopropoxy, butoxy, sec-butoxy, isobutoxy and tert-butoxy, further C₁-C₄-alkylthio such as methylthio, ethylthio, propylthio, isopropylthio, and n-butylthio further C₁-C₄-alkylsulfinyl such as methylsulfinyl, ethylsulfinyl, propylsulfinyl, isopropylsulfinyl, and n-butylsulfinyl, and further C₁-C₄-alkylsulfonyl such as methylsulfonyl, ethylsulfonyl, propylsulfonyl, isopropylsulfonyl, and n-butylsulfonyl.

Accordingly, the terms “Cₙ-Cₘ-haloalkoxy” and “Cₙ-Cₘ-haloalkylthio” (═Cₙ-Cₘ-haloalkylsulfenyl) (and also Cₙ-Cₘ-haloalkylsulfinyl and Cₙ-Cₘ-haloalkylsulfonyl) refer to straight-chain or branched alkyl groups having n to m carbon atoms, e.g. 1 to 10, in particular 1 to 6 or 1 to 4 carbon atoms (as mentioned above) bonded through oxygen or sulfur linkages (or SO (sulfinyl) or S(O)₂ (sulfonyl) linkages), respectively, at any bond in the alkyl group, where some or all of the hydrogen atoms in these groups may be replaced by halogen atoms as mentioned above, for example C₁-C₂-haloalkoxy, such as chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy and pentafluoroethoxy, further C₁-C₂-haloalkylthio, such as chloromethylthio, bromomethylthio, dichloromethylthio, trichloromethylthio, fluoromethylthio, difluoromethylthio, trifluoromethylthio, chlorofluoromethylthio, dichlorofluoromethylthio, chlorodifluoromethylthio, 1-chloroethylthio, 1-bromoethylthio, 1-fluoroethylthio, 2-fluoroethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio and pentafluoroethylthio and the like, further C₁-C₂-haloalkylsulfinyl, such as chloromethylsulfinyl, bromomethylsulfinyl, dichloromethylsulfinyl, trichloromethylsulfinyl, fluoromethylsulfinyl, difluoromethylsulfinyl, trifluoromethylsulfinyl, chlorofluoromethylsulfinyl, dichlorofluoromethylsulfinyl, chlorodifluoromethylsulfinyl, 1-chloroethylsulfinyl, 1-bromoethylsulfinyl, 1-fluoroethylsulfinyl, 2-fluoroethylsulfinyl, 2,2-difluoroethylsulfinyl, 2,2,2-trifluoroethylsulfinyl, 2-chloro-2-fluoroethylsulfinyl, 2-chloro-2,2-difluoroethylsulfinyl, 2,2-dichloro-2-fluoroethylsulfinyl, 2,2,2-trichloroethylsulfinyl and pentafluoroethylsulfinyl and the like, further C₁-C₂-haloalkylsulfonyl, such as chloromethylsulfonyl, bromomethylsulfonyl, dichloromethylsulfonyl, trichloromethylsulfonyl, fluoromethylsulfonyl, difluoromethylsulfonyl, trifluoromethylsulfonyl, chlorofluoromethylsulfonyl, dichlorofluoromethylsulfonyl, chlorodifluoromethylsulfonyl, 1-chloroethylsulfonyl, 1-bromoethylsulfonyl, 1-fluoroethylsulfonyl, 2-fluoroethylsulfonyl, 2,2-difluoroethylsulfonyl, 2,2,2-trifluoroethylsulfonyl, 2-chloro-2-fluoroethylsulfonyl, 2-chloro-2,2-difluoroethylsulfonyl, 2,2-dichloro-2-fluoroethylsulfonyl, 2,2,2-trichloroethylsulfonyl and pentafluoroethylsulfonyl and the like. Similarly the terms C₁-C₂-fluoroalkoxy and C₁-C₂-fluoroalkylthio (and also fluoroalkylsulfinyl and fluoroalkylsulfonyl) refer to C₁-C₂-fluoroalkyl which is bound to the remainder of the molecule via an oxygen atom or a sulfur atom (or a SO (sulfinyl) or a S(O)₂ (sulfonyl) linkage), respectively.

The term “C₂-Cₘ-alkenyl” as used herein intends a branched or unbranched unsaturated hydrocarbon group having 2 to m, e.g. 2 to 10 or 2 to 6 carbon atoms and a double bond in any position, such as ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl.

The term “C₂-Cₘ-alkynyl” as used herein refers to a branched or unbranched unsaturated hydrocarbon group having 2 to m, e.g. 2 to 10 or 2 to 6 carbon atoms and containing at least one triple bond, such as ethynyl, propynyl, 1-butynyl, 2-butynyl, and the like.

The term C₁-C₄-alkoxy-C₁-C₄-alkyl as used herein refers to alkyl having 1 to 4 carbon atoms, wherein one hydrogen atom of the alkyl radical is replaced by a C₁-C₄-alkoxy group.

The term C₁-C₄-alkylthio-C₁-C₄-alkyl as used herein refers to alkyl having 1 to 4 carbon atoms, wherein one hydrogen atom of the alkyl radical is replaced by a C₁-C₄-alkylthio group.

The term C₁-C₆-alkylcarbonyl as used herein refers to a carbonyl group carrying a C₁-C₆-alkyl group.

The term C₁-C₁₀-alkoxycarbonyl or C₁-C₄-alkoxycarbonyl as used herein refers to a carbonyl group carrying a C₁-C₁₀-alkyl group or a C₁-C₄-alkyl group.

The term C₁-C₄-alkylcarbonyloxy refers to a carbonyl group which is bound via an oxygen atom and which carries a C₁-C₄-alkyl group.

The term “C₃-Cₘ-cycloalkyl” as used herein refers to a monocyclic 3- to m-membered saturated cycloaliphatic radical, for example to a monocyclic 3- to 10-membered or 3- to 8-membered saturated cycloaliphatic radical such as cyclopropyl, cyclobutyl, cyclopentyl, cyclohexyl, cycloheptyl, cyclooctyl and cyclodecyl.

The term aryl as used herein refers to an aromatic hydrocarbon radical such as naphthyl or in particular phenyl.

The term aryl-C₁-C₄-alkyl as used herein refers to an aromatic hydrocarbon radical, which is bound to the remainder of the molecule via a C₁-C₄-alkylene group. Examples comprise benzyl, 1-phenylethyl and 2-phenylethyl.

The term “heterocyclyl” as used herein (and also in “heterocyclyl-C₁-C₄-alkyl”) refers to a saturated or partially unsaturated non-aromatic heterocyclic radical having preferably 3 to 7 ring members (“C₃-C₇-heterocyclyl” or “3- to 7-membered heterocyclyl”) and containing 1, 2, 3 or 4 heteroatoms selected from O, N and S and/or heteroatom groups, selected from S═O, S(O)₂ and N—R, with R being H or alkyl, e.g. C₁-C₆-alkyl, as ring members, and optionally additionally containing 1, 2 or 3 CO groups as ring members. Examples for such non-aromatic heterocyclyl rings include azetidinyl, pyrrolidinyl, pyrrolidinonyl, pyrrolidindionyl, pyrazolinyl, pyrazolinonyl, imidazolinyl, imidazolinonyl, imidazolindionyl, pyrrolinyl, pyrrolinonyl, pyrrolindionyl, pyrazolinyl, imidazolinyl, imidazolinonyl, tetrahydrofuranyl, dihydrofuranyl, 1,3-dioxolanyl, dioxolenyl, thiolanyl, dihydrothienyl, oxazolidinyl, isoxazolidinyl, oxazolinyl, isoxazolinyl, thiazolinyl, isothiazolinyl, thiazolidinyl, isothiazolidinyl, oxathiolanyl, piperidinyl, piperidinonyl, piperidindionyl, piperazinyl, pyridinonyl, pyridindionyl, pyridazinonyl, pyridazindionyl, pyrimidinonyl, pyridazindionyl, pyranyl, dihydropyranyl, tetrahydropyranyl, dioxanyl, thiopyranyl, dihydrothiopyranyl, tetrahydrothiopyranyl, morpholinyl, thiazinyl and the like.

The term “heteroaryl” as used herein (and also in “heteroaryl-C₁-C₄-alkyl”) refers to an aromatic heterocyclic radical having preferably 5 or 6 ring members (“C₅-C₆-heteroaryl” or “5- or 6-membered heteroaryl”), wherein 1, 2, 3 or 4 ring members are heteroatoms selected from O, N and S or heteroatom groups, selected from S═O, S(O)₂ or N—R, with R being H or alkyl. Examples for monocyclic 5- or 6-membered heteroaromatic rings include triazinyl, pyrazinyl, pyrimidyl, pyridazinyl, pyridyl, thienyl, furyl, pyrrolyl, pyrazolyl, imidazolyl, triazolyl, tetrazolyl, thiazolyl, oxazolyl, thiadiazolyl, oxadiazolyl, isothiazolyl and isoxazolyl.

The terms heterocyclyl-C₁-C₄-alkyl” and “heteroaryl-C₁-C₄-alkyl” as used herein refer to a non-aromatic or aromatic heterocyclic radical which is bound to the remainder of the molecule via a C₁-C₄-alkylene group, such as methylene, 1,2-ethylene, 1,2- or 1,3-propylene or 1,4-butylene. Typical examples for heteroaryl-C₁-C₄-alkyl are benzyl and 2-phenylethyl. Examples for heterocyclyl-C₁-C₄-alkyl are cyclopropylmethyl, 2-cyclopropylethyl, cyclopentylmethyl, 2-cyclopentylethyl cyclohexylmethyl and 2-cyclohexylethyl.

The term “ethylenically unsaturated ring” as used herein means that the ring contains at least one double bond which may be a C—C double bond but also a double bond containing at least one heteroatom, e.g. C═N or N═N.

With respect to the use according to the invention of the compounds of formula I, particular preference is given to the following meanings of the substituents and variables (R¹, R², R³, R⁴, A, m), in each case on their own or in combination:

In one preferred embodiment of the invention, at least one of the radicals R¹, R², R³ or R⁴ is not hydrogen.

In a more preferred embodiment of the invention, R¹ is not hydrogen.

Preferably, R¹ is selected from halogen, C₁-C₄-alkyl, C₁-C₄-haloalkyl, C₁-C₄-alkoxy and C₁-C₄-haloalkoxy. More preferably, R¹ is selected from C₁-C₂-alkyl, C₁-haloalkyl, C₁-C₂-alkoxy and C₁-haloalkoxy. Even more preferably, R¹ is selected from methyl, methoxy, trifluoromethyl, trichloromethyl, methoxy, trifluoromethoxy, difluoromethoxy, fluoromethoxy and chlorodifluoromethoxy and in particular from methyl, methoxy, and difluoromethoxy. In a specific embodiment of the invention, R¹ is selected from methyl, methoxy, trifluoromethoxy, difluoromethoxy, fluoromethoxy and chlorodifluoromethoxy and more specifically from methyl, methoxy and difluoromethoxy. In another specific embodiment of the invention R¹ is halogen and in particular chlorine. In another specific embodiment of the invention R¹ is selected from C₁-C₄-alkoxy and C₁-C₄-haloalkoxy and more specifically from C₁-C₂-alkoxy, such as methoxy and ethoxy, and C₁₋haloalkoxy, such as trifluoromethoxy, difluoromethoxy, fluoromethoxy and chlorodifluoromethoxy and in particular difluoromethoxy.

R², R³, and R⁴ are preferably selected, independently from each other, from H, C₁-C₄-alkyl, C₁-C₄-haloalkyl, C₁-C₄-alkoxy, C₁-C₄-haloalkoxy and halogen. More preferably R², R³ and R⁴ are selected, independently from each other, from hydrogen, halogen, C₁-C₄-alkyl and C₁-C₄-haloalkyl, especially from hydrogen, halogen, C₁-C₂-alkyl, and C₁-haloalkyl, most preferably, from H, F, Cl, Br, I, CH₃ and CF₃. In a specific embodiment of the invention, two of the radicals R², R³ or R⁴ are hydrogen and the remaining radical is selected from halogen, C₁-C₄-alkyl and C₁-C₄-haloalkyl and preferably from halogen. More specifically, R² and R³ are H and R⁴ is different from H and is preferably halogen. In another specific embodiment of the invention, all three radicals R², R³ and R⁴ are hydrogen.

Preferred embodiments of the radical A:

A. In one preferred embodiment, A is a radical N═CR⁵R⁶.

Preferably, R⁵ and R⁶ are not both hydrogen. More preferably, R⁵ is not hydrogen.

Preferably, R⁵ is OR^{5a}, NR^{5b}R^{5c}, aryl-C₁-C₄-alkyl or C₁-C₁₀-alkyl which may be unsubstituted, partially or fully halogenated and/or may carry 1, 2 or 3 radicals, independently of one another selected from the group consisting of C₁-C₁₀-alkoxy and phenyl, it being possible for phenyl to be unsubstituted, partially or fully halogenated or carry 1, 2 or 3 radicals, independently of one another selected from the group consisting of C₁-C₄-alkyl, C₁-C₄-haloalkyl, C₁-C₄-alkoxy and C₁-C₄-haloalkoxy, wherein R^{5a}, R^{5b} and R^{5c} are as defined above.

Preferably, R⁶ is hydrogen, OR^{6a}, NR^{6b}R^{6c}, aryl-C₁-C₄-alkyl or C₁-C₁₀-alkyl which may be unsubstituted, partially or fully halogenated and/or may carry 1, 2 or 3 radicals, independently of one another selected from the group consisting of C₁-C₁₀-alkoxy and phenyl, it being possible for phenyl to be unsubstituted, partially or fully halogenated or carry 1, 2 or 3 radicals, independently of one another selected from the group consisting of C₁-C₄-alkyl, C₁-C₄-haloalkyl, C₁-C₄-alkoxy and C₁-C₄-haloalkoxy, wherein R^{6a}, R^{6b} and R^{6c} have one of the meanings given above for R⁵, R^{5b} and R^{5c}.

However, following meanings of R^{5a}, R^{5b}, R^{5c}, R^{6a}, R^{6b} and R^{6c} are preferred:

Preferably, R^{5a} is C₁-C₁₀-alkyl which may be unsubstituted, partially or fully halogenated and/or may carry 1, 2 or 3 radicals, independently of one another selected from C₁-C₄-alkoxy, or is C₂-C₁₀-alkenyl or C₂-C₁₀-alkynyl. More preferably, R^{5a} is C₁-C₁₀-alkyl which may be unsubstituted, partially or fully halogenated and/or may carry 1, 2 or 3 radicals, independently of one another selected from C₁-C₄-alkoxy. In particular, R^{5a} is unsubstituted C₁-C₁₀-alkyl.

Preferably, R^{5b} and R^{5c} are, independently from each other, hydrogen, C₁-C₁₀-alkyl which may be unsubstituted, partially or fully halogenated and/or may carry 1, 2 or 3 radicals, independently of one another selected from C₁-C₄-alkoxy, or are C₂-C₁₀-alkenyl or C₂-C₁₀-alkynyl. More preferably, R^{5b} and R^{5c} are, independently from each other, hydrogen or C₁-C₁₀-alkyl which may be unsubstituted, partially or fully halogenated and/or may carry 1, 2 or 3 radicals, independently of one another selected from C₁-C₄-alkoxy. In particular, R^{5b} and R^{5c} are, independently from each other, hydrogen or unsubstituted C₁-C₁₀-alkyl. Preferably, at least one of R^{5b} and R^{5c} is not hydrogen.

Preferably, R^{6a} is C₁-C₁₀-alkyl which may be unsubstituted, partially or fully halogenated and/or may carry 1, 2 or 3 radicals, independently of one another selected from C₁-C₄-alkoxy, or is C₂-C₁₀-alkenyl or C₂-C₁₀-alkynyl. More preferably, R^{6a} is C₁-C₁₀-alkyl which may be unsubstituted, partially or fully halogenated and/or may carry 1, 2 or 3 radicals, independently of one another selected from C₁-C₄-alkoxy. In particular, R^{6a} is unsubstituted C₁-C₁₀-alkyl.

Preferably, R^{6b} and R^{6c} are independently from each other, hydrogen, C₁-C₁₀-alkyl which may be unsubstituted, partially or fully halogenated and/or may carry 1, 2 or 3 radicals, independently of one another selected from C₁-C₄-alkoxy, or is C₂-C₁₀-alkenyl or C₂-C₁₀-alkynyl. More preferably, R^{6b} and R^{6c} are, independently from each other, hydrogen or C₁-C₁₀-alkyl which may be unsubstituted, partially or fully halogenated and/or may carry 1, 2 or 3 radicals, independently of one another selected from C₁-C₄-alkoxy. In particular, R^{6b} and R^{6c} are, independently from each other, hydrogen or un-substituted C₁-C₁₀-alkyl. Preferably, at least one of R^{6b} and R^{6c} is not hydrogen.

A.1 In a more preferred embodiment, A is a radical N═CR⁵R⁶, wherein

R⁵ is C₁-C₁₀-alkyl, preferably C₁-C₆-alkyl, or a radical OR^{5a}, and
R⁶ is a radical OR^{6a},
wherein R^{5a} and R^{6a} have, independently of each other, one of the meanings of R^{5a} given above.

Preferably, R^{5a} and R^{6a} are, independently of each other, C₁-C₁₀-alkyl which may be unsubstituted, partially or fully halogenated and/or may carry 1, 2 or 3 radicals selected from C₁-C₄-alkoxy, or are C₂-C₁₀-alkenyl or C₂-C₁₀-alkynyl.

More preferably, R^{5a} and R^{6a} are, independently of each other, C₁-C₆-alkyl, C₂-C₆-alkenyl, C₂-C₆-alkynyl, C₁-C₄-alkoxy-C₁-C₄-alkyl or C₁-C₄-haloalkyl. Even more preferably, R^{5a} and R^{6a} are, independently of each other, C₁-C₄-alkyl, in particular C₁-C₂-alkyl, C₂-C₆-alkenyl, in particular C₂-C₄-alkenyl, C₂-C₆-alkynyl, in particular C₂-C₄-alkynyl, C₁-C₄-alkoxy-C₁-C₄-alkyl, in particular C₁-C₂-alkoxy-C₁-C₂-alkyl, or C₁-C₄-haloalkyl, in particular C₁-C₂-haloalkyl. In a specific embodiment R^{5a} and R^{6a} are, independently of each other, C₁-C₄-alkyl, in particular C₁-C₂-alkyl.

A.2 Alternatively, in a more preferred embodiment, A is a radical N═CR⁵R⁶, wherein
R⁵ is C₁-C₆-alkyl, which may be unsubstituted, partially or fully halogenated and/or may carry 1, 2 or 3 radicals, selected from the group consisting of cyano, nitro, amino, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, (C₁-C₄-alkoxy)carbonyl, (C₁-C₄-alkyl)amino, di(C₁-C₄-alkyl)amino and C₃-C₈-cycloalkyl, or is phenyl or phenyl-C₁-C₄-alkyl, wherein the phenyl group in the two last-mentioned radicals may be unsubstituted or may carry 1, 2 or 3 substituents, independently of one another selected from the group consisting of halogen, C₁-C₄-alkyl, C₁-C₄-haloalkyl, C₁-C₄-alkoxy and C₁-C₄-haloalkoxy; and
R⁶ is hydrogen, C₁-C₆-alkyl, which may be unsubstituted, partially or fully halogenated and/or may carry 1, 2 or 3 radicals, selected from the group consisting of cyano, nitro, amino, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, (C₁-C₄-alkoxy)carbonyl, (C₁-C₄-alkyl)amino, di(C₁-C₄-alkyl)amino and C₃-C₈-cycloalkyl, or is phenyl or phenyl-C₁-C₄-alkyl, wherein the phenyl group in the two last-mentioned radicals may be unsubstituted or may carry 1, 2 or 3 substituents, independently of one another selected from the group consisting of halogen, C₁-C₄-alkyl, C₁-C₄-haloalkyl, C₁-C₄-alkoxy and C₁-C₄-haloalkoxy.

Preferably,
R⁵ is C₁-C₆-alkyl, C₁-C₄-alkoxy-C₁-C₄-alkyl, C₁-C₄-haloalkyl or phenyl-C₁-C₄-alkyl, wherein phenyl may be unsubstituted, partially or fully halogenated and/or carry 1, 2 or 3 substituents, independently of one another selected from the group consisting of C₁-C₄-alkyl, C₁-C₄-haloalkyl, C₁-C₄-alkoxy and C₁-C₄-haloalkoxy; and
R⁶ is hydrogen, C₁-C₆-alkyl, C₁-C₄-alkoxy-C₁-C₄-alkyl, C₁-C₄-haloalkyl or phenyl-C₁-C₄-alkyl, wherein phenyl may be unsubstituted, partially or fully halogenated and/or carry 1, 2 or 3 substituents, independently of one another selected from the group consisting of C₁-C₄-alkyl, C₁-C₄-haloalkyl, C₁-C₄-alkoxy and C₁-C₄-haloalkoxy.

Specifically, R⁵ and R⁶ are, independently of each other, C₁-C₆-alkyl, in particular C₁-C₄-alkyl, or phenyl-C₁-C₄-alkyl, in particular benzyl. More specifically, R⁵ and R⁶ are, independently of each other, C₁-C₆-alkyl, in particular C₁-C₄-alkyl.

A.3 Alternatively, in a more preferred embodiment, A is a radical N═CR⁵R⁶, wherein
R⁵ is a radical NR^{5b}R^{5c}; and
R⁶ is hydrogen, C₁-C₆-alkyl, which may be unsubstituted, partially or fully halogenated and/or may carry 1, 2 or 3 radicals, selected from the group consisting of cyano, nitro, amino, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, (C₁-C₄-alkoxy)carbonyl, (C₁-C₄-alkyl)amino, di(C₁-C₄-alkyl)amino and C₃-C₈-cycloalkyl, or is phenyl or phenyl-C₁-C₄-alkyl, wherein the phenyl group in the two last-mentioned radicals may by unsubstituted or may carry 1, 2 or 3 substituents, independently of one another selected from the group consisting of halogen, C₁-C₄-alkyl, C₁-C₄-haloalkyl, C₁-C₄-alkoxy and C₁-C₄-haloalkoxy, or is a radical OR^{6a},
   wherein R^{5a} and R^{5b} are, independently of each other, as defined above, and R^{6a} has one of the meanings given above for R^{5a}.

Preferably, R^{5b} and R^{5c} are, independently of each other, hydrogen, C₁-C₁₀-alkyl which may be unsubstituted, partially or fully halogenated and/or may carry 1, 2 or 3 radicals selected from C₁-C₄-alkoxy, or are C₂-C₁₀-alkenyl or C₂-C₁₀-alkynyl.

More preferably, R^{5b} and R^{5c} are, independently of each other, hydrogen, C₁-C₆-alkyl, C₂-C₆-alkenyl, C₂-C₆-alkynyl, C₁-C₄-alkoxy-C₁-C₄-alkyl or C₁-C₄-haloalkyl. Even more preferably, R^{5b} and R^{5c} are, independently of each other, hydrogen, C₁-C₄-alkyl, C₂-C₆-alkenyl, in particular C₂-C₄-alkenyl, C₂-C₆-alkynyl, in particular C₂-C₄-alkynyl, C₁-C₄-alkoxy-C₁-C₄-alkyl, in particular C₁-C₂-alkoxy-C₁-C₂-alkyl, or C₁-C₄-haloalkyl, in particular C₁-C₂-haloalkyl. In a specific embodiment R^{5b} and R^{5c} are, independently of each other, hydrogen or C₁-C₆-alkyl and more specifically hydrogen or C₁-C₄-alkyl.

It is preferred that at least one of the radicals R^{5b} and R^{5c} is not hydrogen.

Preferably, R^{6a} is C₁-C₁₀-alkyl which may be unsubstituted, partially or fully halogenated and/or may carry 1, 2 or 3 radicals selected from C₁-C₄-alkoxy, or is C₂-C₁₀-alkenyl or C₂-C₁₀-alkynyl.

More preferably, R^{6a} is C₁-C₆-alkyl, C₂-C₆-alkenyl, C₂-C₆-alkynyl, C₁-C₄-alkoxy-C₁-C₄-alkyl or C₁-C₄-haloalkyl. Even more preferably, R^{6a} is C₁-C₄-alkyl, in particular C₁-C₂-alkyl, C₂-C₆-alkenyl, in particular C₂-C₄-alkenyl, C₂-C₆-alkynyl, in particular C₂-C₄-alkynyl, C₁-C₄-alkoxy-C₁-C₄-alkyl, in particular C₁-C₂-alkoxy-C₁-C₂-alkyl, or C₁-C₄-haloalkyl, in particular C₁-C₂-haloalkyl. In a specific embodiment R^{6a} is C₁-C₄-alkyl, in particular C₁-C₂-alkyl.

Preferably, R⁶ is hydrogen; C₁-C₆-alkyl, which may be unsubstituted, partially or fully halogenated and/or may carry 1, 2 or 3 radicals, selected from C₁-C₄-alkoxy; phenyl-C₁-C₄-alkyl, wherein the phenyl group may by unsubstituted or may carry 1, 2 or 3 substituents, independently of one another selected from the group consisting of halogen, C₁-C₄-alkyl, C₁-C₄-haloalkyl, C₁-C₄-alkoxy and C₁-C₄-haloalkoxy; or a radical OR^{6a}.

More preferably, R⁶ is hydrogen, C₁-C₆-alkyl or a radical OR^{6a}. Specifically, R⁶ is hydrogen, C₁-C₄-alkyl or a radical OR^{6a}. The preferred embodiments of R^{6a} are as defined above.

B. In another preferred embodiment, A is a radical N═SR⁷R⁸.

Preferably, R⁷ and R⁸ are, independently of each other, aryl, aryl-C₁-C₄-alkyl, C₂-C₆-alkenyl, C₂-C₆-alkynyl, C₁-C₆-alkyl which may be unsubstituted, partially or fully halogenated and/or may carry 1, 2 or 3 radicals, selected from the group consisting of C₁-C₄-alkoxy, or R⁷ and R⁸, together with the sulfur atom they are bound to, form a 5-, 6- or 7-membered saturated or ethylenically unsaturated ring which may be unsubstituted or may carry 1, 2, 3 or 4 substituents selected from halogen and C₁-C₄-alkyl, and which may contain 1 or 2 carbonyl groups and/or heteroatoms selected from N, O and S and/or heteroatom groups selected from SO, SO₂ and NR^{#}, R^{#} being H or C₁-C₄-alkyl, as ring members.

More preferably, R⁷ and R⁸ are, independently of each other, C₁-C₆-alkyl, C₂-C₆-alkenyl, C₂-C₆-alkynyl, C₁-C₄-alkoxy-C₁-C₄-alkyl, C₁-C₄-haloalkyl, phenyl or phenyl-C₁-C₄-alkyl, wherein phenyl in the last two radicals may be unsubstituted, partially or fully halogenated and/or carry 1, 2 or 3 substituents, independently of one another selected from the group consisting of C₁-C₄-alkyl, C₁-C₄-haloalkyl, C₁-C₄-alkoxy and C₁-C₄-haloalkoxy,

or R⁷ and R⁸ together form a moiety (CH₂)ₖ, wherein k is 4, 5 or 6 and wherein 1, 2, 3 or 4 hydrogen atoms may be replaced by C₁-C₄-alkyl or halogen and wherein 1 or 2 non-adjacent CH₂ moieties may be replaced by a carbonyl group, a heteroatom or a heteroatom group, selected from O, S, SO₂ and N—R^{#} with R^{#} being H or C₁-C₄-alkyl.

Even more preferably, R⁷ and R⁸ are, independently of each other, C₁-C₆-alkyl, in particular C₁-C₄-alkyl, C₁-C₄-alkoxy-C₁-C₄-alkyl, in particular C₁-C₂-alkoxy-C₁-C₂-alkyl, C₁-C₄-haloalkyl, phenyl, or phenyl-C₁-C₄-alkyl, in particular benzyl, or R⁷ and R⁸ together form a moiety (CH₂)ₖ, wherein k is 4 or 5. Specifically, R⁷ and R⁸ are C₁-C₆-alkyl, in particular C₁-C₄-alkyl, or together form a moiety (CH₂)ₖ, wherein k is 4 or 5, in particular 4.

C. In another preferred embodiment, A is a radical of the formula NR¹⁰—C(═X)—R⁹, wherein X, R⁹ and R¹⁰ are as defined above.

C.1 In a more preferred embodiment, A is a radical of the formula NR¹⁰—CO—R⁹, wherein R⁹ and R¹⁰ are as defined above.

Preferably, R⁹ is selected from the group consisting of hydrogen, OR^{9a}, NR^{9b}R^{9c}, aryl, aryl-C₁-C₄-alkyl, C₁-C₁₀-alkyl, C₂-C₁₀-alkenyl and C₂-C₁₀-alkynyl, wherein C₁-C₁₀-alkyl, C₂-C₁₀-alkenyl and C₂-C₁₀-alkynyl may be unsubstituted, partially or fully halogenated and/or may carry 1, 2 or 3 radicals selected from C₁-C₁₀-alkoxy,

and wherein
R^{9a} is C₁-C₁₀-alkyl, C₃-C₁₀-cycloalkyl, C₂-C₁₀-alkenyl, C₂-C₁₀-alkynyl, aryl, aryl-C₁-C₄-alkyl, heteroaryl or heteroaryl-C₁-C₄-alkyl, heterocyclyl or heterocyclyl-C₁-C₄-alkyl,
R^{9b}, R^{9c}, independently from each other, are selected from hydrogen, C₁-C₁₀-alkyl, C₃-C₁₀-cycloalkyl, C₂-C₁₀-alkenyl, C₂-C₁₀-alkynyl, aryl and aryl-C₁-C₄-alkyl,
   wherein C₁-C₁₀-alkyl, C₃-C₁₀-cycloalkyl, C₂-C₁₀-alkenyl and C₂-C₁₀-alkynyl in R^{9a}, R^{9b} and R^{9c} may be unsubstituted, partially or fully halogenated and/or may carry 1, 2 or 3 radicals selected from C₁-C₄-alkoxy,
   and wherein the carbon atoms of aryl, heteroaryl and heterocyclyl moieties in R⁹, R^{9a}, R^{9b} and R^{9c} may be unsubstituted or may carry 1, 2 or 3 substituents, independently of one another selected from the group consisting of halogen, C₁-C₄-alkyl, C₁-C₄-haloalkyl, C₁-C₄-alkoxy and C₁-C₄-haloalkoxy.

Preferably, R¹⁰ is selected from the group consisting of hydrogen, C(═O)—R¹⁶, C₁-C₁₀-alkyl, C₂-C₆-alkenyl, C₂-C₁₀-alkynyl and C₁-C₁₀-alkoxy, wherein the four last-mentioned radicals may be unsubstituted, partially or fully halogenated and/or may carry 1, 2 or 3 radicals, independently of one another each selected from C₁-C₁₀-alkoxy.

Alternatively, it is preferred that R⁹ and R¹⁰ together with the adjacent nitrogen and carbon atoms form a saturated or ethylenically unsaturated 5 to 10-membered ring, optionally substituted by 1, 2, 3 or 4 radicals selected from C₁-C₅-alkyl and halogen, wherein the ring may contain, in addition to the nitrogen and carbon ring members, 1, 2 or 3 heteroatoms or heteroatom-containing groups as ring members selected from the group consisting of nitrogen, oxygen, sulfur, a group CO, SO, SO₂ or N—R¹⁷.

C.1.1 In an even more preferred embodiment, A is a radical of the formula NR¹⁰—C(═O)—R⁹, wherein
R¹⁰ is selected from hydrogen, C₁-C₆-alkyl, C₂-C₆-alkenyl, C₂-C₆-alkynyl, C₁-C₆-alkoxy, C₁-C₄-alkoxy-C₁-C₄-alkyl, C₁-C₄-haloalkyl and acetyl; and
R⁹ is selected from C₁-C₆-alkyl, C₂-C₆-alkenyl, C₂-C₆-alkynyl, C₁-C₄-alkoxy-C₁-C₄-alkyl, C₁-C₄-haloalkyl, phenyl and phenyl-C₁-C₄-alkyl, wherein phenyl in the last two radicals may be unsubstituted, partially or fully halogenated and/or carry 1, 2 or 3 substituents, independently of one another selected from the group consisting of C₁-C₄-alkyl, C₁-C₄-haloalkyl, C₁-C₄-alkoxy and C₁-C₄-haloalkoxy, or
R¹⁰ and R⁹ together form a moiety of the (CH₂)ₚ, wherein p is 3, 4 or 5 and wherein 1, 2, 3 or 4 hydrogen atoms may be replaced by C₁-C₄-alkyl or halogen and wherein 1 or 2 non-adjacent CH₂ moieties may be replaced by a carbonyl group, a heteroatom or a heteroatom group, selected from O, S, SO₂ and N—R^{#} with R^{#} being H or C₁-C₄-alkyl.

In a specific embodiment,
R¹⁰ is selected from hydrogen, C₁-C₄-alkyl, C₂-C₆-alkynyl and C₁-C₄-alkoxy and in particular from C₁-C₄-alkyl; and
R⁹ is selected from C₁-C₄-alkyl, C₂-C₆-alkenyl, C₁-C₄-haloalkyl and phenyl, or
R¹⁰ and R⁹ together form a moiety of the (CH₂)ₚ, wherein p is 3 or 4.

In an alternative specific embodiment,
R¹⁰ is selected from hydrogen and C₁-C₆-alkyl; and
R⁹ is selected from C₁-C₆-alkyl and C₁-C₄-haloalkyl.

C.1.2 In another even more preferred embodiment, A is a radical of the formula NR¹⁰—C(═O)—R⁹, wherein
R¹⁰ is selected from hydrogen, C₁-C₆-alkyl, C₂-C₆-alkenyl, C₂-C₆-alkynyl, C₁-C₆-alkoxy, C₁-C₄-alkoxy-C₁-C₄-alkyl and C₁-C₄-haloalkyl; and
R⁹ is OR^{9a} or a radical NR^{9b}R^{9c},
   wherein R^{9a}, R^{9b}, and R^{9c} are as defined above.

Preferably, R^{9a} is C₁-C₁₀-alkyl which may be unsubstituted, partially or fully halogenated and/or may carry 1, 2 or 3 radicals selected from C₁-C₄-alkoxy, or is C₂-C₁₀-alkenyl, C₂-C₁₀-alkynyl, aryl or aryl-C₁-C₄-alkyl. More preferably, R^{9a} is C₁-C₆-alkyl, C₂-C₆-alkenyl, C₂-C₆-alkynyl, C₁-C₄-alkoxy-C₁-C₄-alkyl, C₁-C₄-haloalkyl, phenyl or phenyl-C₁-C₄-alkyl. Specifically, R^{9a} is C₂-C₆-alkenyl, in particular vinyl or allyl, phenyl or phenyl-C₁-C₄-alkyl, in particular benzyl.

Preferably, R^{9b} and R^{9c} are, independently of each other, hydrogen, C₁-C₁₀-alkyl which may be unsubstituted, partially or fully halogenated and/or may carry 1, 2 or 3 radicals selected from C₁-C₄-alkoxy, or are C₂-C₁₀-alkenyl, C₂-C₁₀-alkynyl, aryl or aryl-C₁-C₄-alkyl. More preferably, R^{9b} and R^{9c} are, independently of each other, hydrogen, C₁-C₆-alkyl, C₂-C₆-alkenyl, C₂-C₆-alkynyl, C₁-C₄-alkoxy-C₁-C₄-alkyl, C₁-C₄-haloalkyl, phenyl or phenyl-C₁-C₄-alkyl. It is preferred that R^{9b} and R^{9c} are not both hydrogen. Specifically, one of R^{9b} and R^{9c} is hydrogen and the other radical is C₁-C₆-alkyl or phenyl.

In a specific embodiment, R¹⁰ is selected from hydrogen and C₁-C₄-alkyl, more specifically from hydrogen and C₁-C₂-alkyl.

D. In another preferred embodiment, A is an N-bound 5-, 6- or 7-membered aromatic or non-aromatic unsaturated heterocycle and which additionally may contain 1, 2, or 3 further heteroatoms or heteroatom groups, selected from O, S, SO, SO₂, N, and NR¹², and/or 1, 2 or 3 carbonyl groups as ring members and which may carry 1, 2, 3 or 4 radicals R¹³, where R¹³ is as defined above. Preferably, at least one double bond is positioned α to the nitrogen atom through which the radical A is bound. In case the heterocycle contains 1, 2 or 3 further heteroatoms or heteroatom groups as ring members, it is preferred that these are selected from N and NR¹².

In a more preferred embodiment, A is an N-bound 5-membered aromatic heterocycle which additionally may contain 1, 2, or 3 further nitrogen atoms as ring members and which may carry 1, 2, 3 or 4 radicals R¹³, where R¹³ is as defined above.

Preferably, R¹³ is independently selected from halogen and C₁-C₄-alkyl which may be partially or fully halogenated and/or may be substituted by 1, 2 or 3 radicals selected from OH, C₁-C₄-alkoxy and C₁-C₄-alkylcarbonyloxy.

Even more preferably, A is 1-pyrrolyl, 1-pyrazolyl, 1-imidazolyl or [1,2,4]-triazol-1-yl, where the heterocycle may be unsubstituted or may carry 1, 2 or 3 substituents, specifically 1 substituent, selected from halogen and C₁-C₄-alkyl, wherein C₁-C₄-alkyl may be unsubstituted or may be substituted by hydroxy or acetyloxy. In particular, A is 1-pyrrolyl which may be unsubstituted or may carry 1, 2 or 3 substituents, specifically 1 substituent, selected from halogen and C₁-C₄-alkyl, wherein C₁-C₄-alkyl may be unsubstituted or may be substituted by hydroxy or acetyloxy.

In a particularly preferred embodiment, A is a radical of the formula NR¹⁰—CO—R⁹, wherein R¹⁰ is selected from hydrogen, C₁-C₄-alkyl, C₂-C₆-alkynyl and C₁-C₄-alkoxy and R⁹ is selected from C₁-C₄-alkyl, C₂-C₆-alkenyl, C₁-C₄-haloalkyl and phenyl; or

A is a radical of the formula NR¹⁰—CO—R⁹, wherein R¹⁰ and R⁹ together form a moiety of the (CH₂)ₚ, wherein p is 3 or 4; or
A is a radical of the formula NR¹⁰—CO—OR^{9a}, wherein R^{9a} is C₁-C₆-alkyl, C₂-C₆-alkenyl, C₂-C₆-alkynyl, C₁-C₄-alkoxy-C₁-C₄-alkyl, C₁-C₄-haloalkyl, phenyl or phenyl-C₁-C₄-alkyl and R¹⁰ is hydrogen or C₁-C₄-alkyl; or
A is a radical of the formula NR¹⁰—CO—NR^{9b}R^{9c}, wherein one of R^{9b} and R^{9c} is hydrogen and the other radical is C₁-C₆-alkyl or phenyl and R¹⁰ is hydrogen or C₁-C₄-alkyl; or
A is a radical of the formula N═CR⁵R⁶, wherein R⁵ is C₁-C₆-alkyl, C₁-C₄-alkoxy-C₁-C₄-alkyl, C₁-C₄-haloalkyl or phenyl-C₁-C₄-alkyl and R⁶ is hydrogen, C₁-C₆-alkyl, C₁-C₄-alkoxy-C₁-C₄-alkyl, C₁-C₄-haloalkyl or phenyl-C₁-C₄-alkyl; or
A is a radical of the formula N═CR⁵OR^{6a}, wherein R⁵ is C₁-C₆-alkyl and R^{6a} is C₁-C₆-alkyl, C₂-C₆-alkenyl, C₂-C₆-alkynyl, C₁-C₄-alkoxy-C₁-C₄-alkyl or C₁-C₄-haloalkyl; or
A is a radical of the formula N═C(OR^{5a})₂, wherein R^{5a} is C₁-C₆-alkyl, C₂-C₆-alkenyl, C₂-C₆-alkynyl, C₁-C₄-alkoxy-C₁-C₄-alkyl or C₁-C₄-haloalkyl; or
A is a radical of the formula N═C(NR^{5b}R^{5c})(OR^{6a}), wherein R^{5b} and R^{5c} are, independently of each other, hydrogen, C₁-C₆-alkyl, C₂-C₆-alkenyl, C₂-C₆-alkynyl, C₁-C₄-alkoxy-C₁-C₄-alkyl or C₁-C₄-haloalkyl and R^{6a} is C₁-C₆-alkyl, C₂-C₆-alkenyl, C₂-C₆-alkynyl, C₁-C₄-alkoxy-C₁-C₄-alkyl or C₁-C₄-haloalkyl; or
A is a radical of the formula N═SR⁷R⁸, wherein R⁷ and R⁸ are, independently of each other, C₁-C₆-alkyl, C₁-C₄-alkoxy-C₁-C₄-alkyl, C₁-C₄-haloalkyl, phenyl, phenyl-C₁-C₄-alkyl or R⁷ and R⁸ together form a moiety (CH₂)ₖ, wherein k is 4, 5 or 6; or
A is an N-bound 5-membered aromatic heterocycle which additionally may contain 1, 2, or 3 further nitrogen atoms as ring members and which may carry 1, 2, 3 or 4 radicals R¹³, where R¹³ is as defined above.

In a specific embodiment of the invention, A is a radical of the formula NR¹⁰—CO—R⁹, wherein R¹⁰ is selected from hydrogen, C₁-C₄-alkyl and C₂-C₆-alkynyl; and R⁹ is selected from C₁-C₄-alkyl, C₂-C₆-alkenyl, C₁-C₄-haloalkyl and phenyl, or R¹⁰ and R⁹ together form a moiety of the (CH₂)ₚ, wherein p is 3 or 4; or A is an N-bound 5-membered aromatic heterocycle which additionally may contain 1, 2, or 3 further nitrogen atoms as ring members and which may carry 1, 2, 3 or 4 radicals R¹³, where R¹³ is as defined above. Preferred embodiments of R¹⁰, R⁹, R¹³ and of the N-bound 5-membered aromatic heterocycle are as defined above.

In formula I the variable m is preferably 2. These compounds are also referred to as compounds Ia. Compounds I, where m is 0 are also referred to as compounds Ib, while compounds I, where m is 1, are also referred to as compounds Ic.

Examples of preferred compounds which are represented by the formulae Ia, Ib and Ic are the individual compounds compiled in the tables 1 to 264 below, where the variable A has the meanings given in one row of table A and the variables R¹, R², R³ and R⁴ have the meanings given in the respective table.

TABLE A

| No. | A |
|---|---|
| A-1 | N═C(OCH₃)(NH₂) |
| A-2 | N═C(OCH₃)(NH(CH₃)) |
| A-3 | N═C(OCH₃)(NH(C₂H₅)) |
| A-4 | N═C(OCH₃)(N(CH₃)₂) |
| A-5 | N═C(OCH₃)(N(CH₃)(C₂H₅)) |
| A-6 | N═C(OCH₃)(N(C₂H₅)₂) |
| A-7 | N═C(OCH₃)(NH(CH₂CH₂CH₃)) |
| A-8 | N═C(OCH₃)(N(CH₃)(CH₂CH₂CH₃)) |
| A-9 | N═C(OCH₃)(N(C₂H₅)(CH₂CH₂CH₃)) |
| A-10 | N═C(OCH₃)(NH(CH(CH₃)₂)) |
| A-11 | N═C(OCH₃)(N(CH₃)(CH(CH₃)₂)) |
| A-12 | N═C(OCH₃)(N(C₂H₅)(CH(CH₃)₂)) |
| A-13 | N═C(OCH₃)(NH(C(CH₃)₃)) |
| A-14 | N═C(OCH₃)(N(CH₃)(C(CH₃)₃)) |
| A-15 | N═C(OCH₃)(N(C₂H₅)(C(CH₃)₃)) |
| A-16 | N═C(OCH₃)(NH(propargyl)) |
| A-17 | N═C(OCH₃)(N(CH₃)(propargyl)) |
| A-18 | N═C(OCH₃)(N(C₂H₅)(propargyl)) |
| A-19 | N═C(OCH₃)(NH(CH₂CH═CH₂)) |
| A-20 | N═C(OCH₃)(N(CH₃)(CH₂CH═CH₂)) |
| A-21 | N═C(OCH₃)(N(C₂H₅)(CH₂CH═CH₂)) |
| A-22 | N═C(OC₂H₅)(NH₂) |
| A-23 | N═C(OC₂H₅)(NH(CH₃)) |
| A-24 | N═C(OC₂H₅)(NH(C₂H₅)) |
| A-25 | N═C(OC₂H₅)(N(CH₃)₂) |
| A-26 | N═C(OC₂H₅)(N(CH₃)(C₂H₅)) |
| A-27 | N═C(OC₂H₅)(N(C₂H₅)₂) |
| A-28 | N═C(OC₂H₅)(NH(CH₂CH₂CH₃)) |
| A-29 | N═C(OC₂H₅)(N(CH₃)(CH₂CH₂CH₃)) |
| A-30 | N═C(OC₂H₅)(N(C₂H₅)(CH₂CH₂CH₃)) |
| A-31 | N═C(OC₂H₅)(NH(CH(CH₃)₂)) |
| A-32 | N═C(OC₂H₅)(N(CH₃)(CH(CH₃)₂)) |
| A-33 | N═C(OC₂H₅)(N(C₂H₅)(CH(CH₃)₂)) |
| A-34 | N═C(OC₂H₅)(NH(C(CH₃)₃)) |
| A-35 | N═C(OC₂H₅)(N(CH₃)(C(CH₃)₃)) |
| A-36 | N═C(OC₂H₅)(N(C₂H₅)(C(CH₃)₃)) |
| A-37 | N═C(OC₂H₅)(NH(propargyl)) |
| A-38 | N═C(OC₂H₅)(N(CH₃)(propargyl)) |
| A-39 | N═C(OC₂H₅)(N(C₂H₅)(propargyl)) |
| A-40 | N═C(OC₂H₅)(NH(CH₂CH═CH₂)) |
| A-41 | N═C(OC₂H₅)(N(CH₃)(CH₂CH═CH₂)) |
| A-42 | N═C(OC₂H₅)(N(C₂H₅)(CH₂CH═CH₂)) |
| A-43 | N═C(O-propargyl)(NH₂) |
| A-44 | N═C(O-propargyl)(NH(CH₃)) |
| A-45 | N═C(O-propargyl)(NH(C₂H₅)) |
| A-46 | N═C(O-propargyl)(N(CH₃)₂) |
| A-47 | N═C(O-propargyl)(N(CH₃)(C₂H₅)) |
| A-48 | N═C(O-propargyl)(N(C₂H₅)₂) |
| A-49 | N═C(O-propargyl)(NH(CH₂CH₂CH₃)) |
| A-50 | N═C(O-propargyl)(N(CH₃)(CH₂CH₂CH₃)) |
| A-51 | N═C(O-propargyl)(N(C₂H₅)(CH₂CH₂CH₃)) |
| A-52 | N═C(O-propargyl)(NH(CH(CH₃)₂)) |
| A-53 | N═C(O-propargyl)(N(CH₃)(CH(CH₃)₂)) |
| A-54 | N═C(O-propargyl)(N(C₂H₅)(CH(CH₃)₂)) |
| A-55 | N═C(O-propargyl)(NH(C(CH₃)₃)) |
| A-56 | N═C(O-propargyl)(N(CH₃)(C(CH₃)₃)) |
| A-57 | N═C(O-propargyl)(N(C₂H₅)(C(CH₃)₃)) |
| A-58 | N═C(O-propargyl)(NH(propargyl)) |
| A-59 | N═C(O-propargyl)(N(CH₃)(propargyl)) |
| A-60 | N═C(O-propargyl)(N(C₂H₅)(propargyl)) |
| A-61 | N═C(O-propargyl)(NH(CH₂CH═CH₂)) |
| A-62 | N═C(O-propargyl)(N(CH₃)(CH₂CH═CH₂)) |
| A-63 | N═C(O-propargyl)(N(C₂H₅)(CH₂CH═CH₂)) |
| A-64 | N═C(OCH₃)(CH₃) |
| A-65 | N═C(OCH₃)(C₂H₅) |
| A-66 | N═C(OCH₃)(CH₂CH₂CH₃) |
| A-67 | N═C(OCH₃)(CH(CH₃)₂) |
| A-68 | N═C(OC₂H₅)(CH₃) |
| A-69 | N═C(OC₂H₅)(C₂H₅) |
| A-70 | N═C(OC₂H₅)(CH₂CH₂CH₃) |
| A-71 | N═C(OC₂H₅)(CH(CH₃)₂) |
| A-72 | N═C(OCH₂CH₂CH₃)(CH₃) |
| A-73 | N═C(OCH₂CH₂CH₃)(C₂H₅) |
| A-74 | N═C(OCH₂CH₂CH₃)(CH₂CH₂CH₃) |
| A-75 | N═C(OCH₂CH₂CH₃)(CH(CH₃)₂) |
| A-76 | N═C(OCH(CH₃)₂)(CH₃) |
| A-77 | N═C(OCH(CH₃)₂)(C₂H₅) |
| A-78 | N═C(OCH(CH₃)₂)(CH₂CH₂CH₃) |
| A-79 | N═C(OCH(CH₃)₂)(CH(CH₃)₂) |
| A-80 | N═C(O-propargyl)(CH₃) |
| A-81 | N═C(O-propargyl)(C₂H₅) |
| A-82 | N═C(O-propargyl)(CH₂CH₂CH₃) |
| A-83 | N═C(O-propargyl)(CH(CH₃)₂) |
| A-84 | N═C(O—CH₂CH═CH₂)(CH₃) |
| A-85 | N═C(O—CH₂CH═CH₂)(C₂H₅) |
| A-86 | N═C(O—CH₂CH═CH₂)(CH₂CH₂CH₃) |
| A-87 | N═C(O—CH₂CH═CH₂)(CH(CH₃)₂) |
| A-88 | N═C(OCH₃)₂ |
| A-89 | N═C(OC₂H₅)₂ |
| A-90 | N═C(OCH₂CH₂CH₃)₂ |
| A-91 | N═C(OCH(CH₃)₂)₂ |
| A-92 | N═C(OCH₂CH₂CH₂CH₃)₂ |
| A-93 | N═C(OCH(CH₃)C₂H₅)₂ |
| A-94 | N═C(OCH₂CH(CH₃)₂)₂ |
| A-95 | N═C(OC(CH₃)₃)₂ |
| A-96 | N═C(O-propargyl)₂ |
| A-97 | N═C(O—CH₂CH═CH₂)₂ |
| A-98 | N═C(O—CH₂OCH₃)₂ |
| A-99 | N═C(O—C₂H₄OCH₃)₂ |
| A-100 | N═C(O—CH₂CF₃)₂ |
| A-101 | N═C(O—C₂H₄CF₃)₂ |
| A-102 | N═C(O—CH₂CCl₃)₂ |
| A-103 | N═C(O—C₂H₄CCl₃)₂ |
| A-104 | N═C(CH₃)(CH₃) |
| A-105 | N═C(CH₃)(C₂H₅) |
| A-106 | N═C(CH₃)(CH₂CH₂CH₃) |
| A-107 | N═C(CH₃)(CH(CH₃)₂) |
| A-108 | N═C(CH₃)(C(CH₃)₃) |
| A-109 | N═C(C₂H₅)(C₂H₅) |
| A-110 | N═C(C₂H₅)(CH₂CH₂CH₃) |
| A-111 | N═C(C₂H₅)(CH(CH₃)₂) |
| A-112 | N═C(C₂H₅)(C(CH₃)₃) |
| A-113 | N═C(CH₂CH₂CH₃)(CH₂CH₂CH₃) |
| A-114 | N═C(CH₂CH₂CH₃)(CH(CH₃)₂) |
| A-115 | N═C(CH₂CH₂CH₃)(C (CH₃)₃) |
| A-116 | N═C(CH(CH₃)₂)(CH(CH₃)₂) |
| A-117 | N═C(CH(CH₃)₂)(C(CH₃)₃) |
| A-118 | N═(C(CH₃)₃)₂ |
| A-119 | N═C(NH₂)H |
| A-120 | N═C(N(CH₃)₂)H |
| A-121 | N═C(N(C₂H₅)₂)H |
| A-122 | N═C(N(CH₂CH₂CH₃)₂)H |
| A-123 | N═C(N(CH(CH₃)₂)₂)H |
| A-124 | N═C(N(propargyl)₂)H |
| A-125 | N═C(N(CH₂CH═CH₂)₂)H |
| A-126 | N═C(NH₂)(CH₃) |
| A-127 | N═C(N(CH₃)₂)CH₃ |
| A-128 | N═C(N(C₂H₅)₂)(CH₃) |
| A-129 | N═C(N(CH₂CH₂CH₃)₂)(CH₃) |
| A-130 | N═C(N(CH(CH₃)₂)₂)(CH₃) |
| A-131 | N═C(N(propargyl)₂)(CH₃) |
| A-132 | N═C(N(CH₂CH═CH₂)₂)(CH₃) |
| A-133 | N═C(NH₂)(C₂H₅) |
| A-134 | N═C(N(CH₃)₂)(C₂H₅) |
| A-135 | N═C(N(C₂H₅)₂)(C₂H₅) |
| A-136 | N═C(N(CH₂CH₂CH₃)₂)(C₂H₅) |
| A-137 | N═C(N(CH(CH₃)₂)₂)(C₂H₅) |
| A-138 | N═C(N(propargyl)₂)(C₂H₅) |
| A-139 | N═C(N(CH₂CH═CH₂)₂)(C₂H₅) |
| A-140 | N═C(NH₂)(C(CH₃)₃) |
| A-141 | N═C(N(CH₃)₂)(C(CH₃)₃) |
| A-142 | N═C(N(C₂H₅)₂)(C(CH₃)₃) |
| A-143 | N═C(N(CH₂CH₂CH₃)₂)(C(CH₃)₃) |
| A-144 | N═C(N(CH(CH₃)₂)₂)(C(CH₃)₃) |
| A-145 | N═C(N(propargyl)₂)(C(CH₃)₃) |
| A-146 | N═C(N(CH₂CH═CH₂)₂)(C(CH₃)₃) |
| A-147 | N═S(CH₃)₂ |
| A-148 | N═S(C₂H₅)(CH₃) |
| A-149 | N═S(CH₂CH₂CH₃)(CH₃) |
| A-150 | N═S(CH(CH₃)₂)(CH₃) |
| A-151 | N═S(CF₃)(CH₃) |
| A-152 | N═S(C₂H₅)₂ |
| A-153 | N═S(CH₂CH₂CH₃)(C₂H₅) |
| A-154 | N═S(CH(CH₃)₂)(C₂H₅) |
| A-155 | N═S(CF₃)(C₂H₅) |
| A-156 | N═S(CH₂CH₂CH₃)₂ |
| A-157 | N═S(CH(CH₃)₂)(CH₂CH₂CH₃) |
| A-158 | N═S(CF₃)(CH₂CH₂CH₃) |
| A-159 | N═S(CH(CH₃)₂)₂ |
| A-160 | N═S(CF₃)(CH(CH₃)₂) |
| A-161 | N═S(CF₃)₂ |
| A-162 | 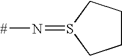 |
| A-163 | 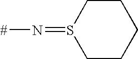 |
| A-164 | N(H)C(O)CH₃ |
| A-165 | N(H)C(O)C₂H₅ |
| A-166 | N(H)C(O)CH₂CH₂CH₃ |
| A-167 | N(H)C(O)CH(CH₃)₂ |
| A-168 | N(H)C(O)C(CH₃)₃ |
| A-169 | N(H)C(O)CF₃ |
| A-170 | N(H)C(O)CH═CH₂ |
| A-171 | N(H)C(O)C₆H₅ |
| A-172 | N(CH₃)C(O)CH₃ |
| A-173 | N(CH₃)C(O)C₂H₅ |
| A-174 | N(CH₃)C(O)CH₂CH₂CH₃ |
| A-175 | N(CH₃)C(O)CH(CH₃)₂ |
| A-176 | N(CH₃)C(O)C(CH₃)₃ |
| A-177 | N(CH₃)C(O)CF₃ |
| A-178 | N(CH₃)C(O)CH═CH₂ |
| A-179 | N(CH₃)C(O)C₆H₅ |
| A-180 | N(C₂H₅)C(O)CH₃ |
| A-181 | N(C₂H₅)C(O)C₂H₅ |
| A-182 | N(C₂H₅)C(O)CH₂CH₂CH₃ |
| A-183 | N(C₂H₅)C(O)CH(CH₃)₂ |
| A-184 | N(C₂H₅)C(O)C(CH₃)₃ |
| A-185 | N(C₂H₅)C(O)CF₃ |
| A-186 | N(C₂H₅)C(O)CH═CH₂ |
| A-187 | N(C₂H₅)C(O)C₆H₅ |
| A-188 | N(CH(CH₃)₂)C(O)CH₃ |
| A-189 | N(CH(CH₃)₂)C(O)C₂H₅ |
| A-190 | N(CH(CH₃)₂)C(O)CH₂CH₂CH₃ |
| A-191 | N(CH(CH₃)₂)C(O)CH(CH₃)₂ |
| A-192 | N(CH(CH₃)₂)C(O)C(CH₃)₃ |
| A-193 | N(CH(CH₃)₂)C(O)CF₃ |
| A-194 | N(CH(CH₃)₂)C(O)CH═CH₂ |
| A-195 | N(CH(CH₃)₂)C(O)C₆H₅ |
| A-196 | N(propargyl)C(O)CH₃ |
| A-197 | N(propargyl)C(O)C₂H₅ |
| A-198 | N(propargyl)C(O)CH₂CH₂CH₃ |
| A-199 | N(propargyl)C(O)CH(CH₃)₂ |
| A-200 | N(propargyl)C(O)C(CH₃)₃ |
| A-201 | N(propargyl)C(O)CF₃ |
| A-202 | N(propargyl)C(O)CH═CH₂ |
| A-203 | N(propargyl)C(O)C₆H₅ |
| A-204 | N(C(O)CH₃)C(O)CH₃ |
| A-205 | N(C(O)CH₃)C(O)C₂H₅ |
| A-206 | N(C(O)CH₃)C(O)CH₂CH₂CH₃ |
| A-207 | N(C(O)CH₃)C(O)CH(CH₃)₂ |
| A-208 | N(C(O)CH₃)C(O)C(CH₃)₃ |
| A-209 | N(C(O)CH₃)C(O)CF₃ |
| A-210 | N(C(O)CH₃)C(O)CH═CH₂ |
| A-211 | N(C(O)CH₃)C(O)C₆H₅ |
| A-212 | N(OCH₃)C(O)CH₃ |
| A-213 | N(OCH₃)C(O)C₂H₅ |
| A-214 | N(OCH₃)C(O)CH₂CH₂CH₃ |
| A-215 | N(OCH₃)C(O)CH(CH₃)₂ |
| A-216 | N(OCH₃)C(O)C(CH₃)₃ |
| A-217 | N(OCH₃)C(O)CF₃ |
| A-218 | N(OCH₃)C(O)CH═CH₂ |
| A-219 | N(OCH₃)C(O)C₆H₅ |
| A-220 | 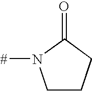 |
| A-221 | 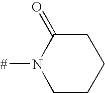 |
| A-222 | N(H)C(O)OCH₃ |
| A-223 | N(CH₃)C(O)OCH₃ |
| A-224 | N(C₂H₅)C(O)OCH₃ |
| A-225 | N(propargyl)C(O)OCH₃ |
| A-226 | N(H)C(O)OC₂H₅ |
| A-227 | N(CH₃)C(O)OC₂H₅ |
| A-228 | N(C₂H₅)C(O)OC₂H₅ |
| A-229 | N(propargyl)C(O)OC₂H₅ |
| A-230 | N(H)C(O)OCH₂CH₂CH₃ |
| A-231 | N(CH₃)C(O)OCH₂CH₂CH₃ |
| A-232 | N(C₂H₅)C(O)OCH₂CH₂CH₃ |
| A-233 | N(propargyl)C(O)OCH₂CH₂CH₃ |
| A-234 | N(H)C(O)OCH(CH₃)₂ |
| A-235 | N(CH₃)C(O)OCH(CH₃)₂ |
| A-236 | N(C₂H₅)C(O)OCH(CH₃)₂ |
| A-237 | N(propargyl)C(O)OCH(CH₃)₂ |
| A-238 | N(H)C(O)OCH₂CH═CH₂ |
| A-239 | N(CH₃)C(O)OCH₂CH═CH₂ |
| A-240 | N(C₂H₅)C(O)OCH₂CH═CH₂ |
| A-241 | N(propargyl)C(O)OCH₂CH═CH₂ |
| A-242 | N(H)C(O)OC₆H₅ |
| A-243 | N(CH₃)C(O)OC₆H₅ |
| A-244 | N(C₂H₅)C(O)OC₆H₅ |
| A-245 | N(propargyl)C(O)OC₆H₅ |
| A-246 | N(H)C(O)OCH₂C₆H₅ |
| A-247 | N(CH₃)C(O)OCH₂C₆H₅ |
| A-248 | N(C₂H₅)C(O)OCH₂C₆H₅ |
| A-249 | N(propargyl)C(O)OCH₂C₆H₅ |
| A-250 | N(H)C(O)NH₂ |
| A-251 | N(H)C(O)NH(CH₃) |
| A-252 | N(H)C(O)NH(C₂H₅) |
| A-253 | N(H)C(O)N(CH₃)₂ |
| A-254 | N(H)C(O)N(CH₃)(C₂H₅) |
| A-255 | N(H)C(O)N(C₂H₅)₂ |
| A-256 | N(H)C(O)NH(CH₂CH₂CH₃) |
| A-257 | N(H)C(O)N(CH₃)(CH₂CH₂CH₃) |
| A-258 | N(H)C(O)N(C₂H₅)(CH₂CH₂CH₃) |
| A-259 | N(H)C(O)NH(CH(CH₃)₂) |
| A-260 | N(H)C(O)N(CH₃)(CH(CH₃)₂) |
| A-261 | N(H)C(O)N(C₂H₅)(CH(CH₃)₂) |
| A-262 | N(H)C(O)NH(CH(CH₃)(C₂H₅)) |
| A-263 | N(H)C(O)N(CH₃)(CH(CH₃)(C₂H₅)) |
| A-264 | N(H)C(O)N(C₂H₅)(CH(CH₃)(C₂H₅)) |
| A-265 | N(H)C(O)NH(propargyl) |
| A-266 | N(H)C(O)N(CH₃)(propargyl) |
| A-267 | N(H)C(O)N(C₂H₅)(propargyl) |
| A-268 | N(H)C(O)NH(CH₂CH═CH₂) |
| A-269 | N(H)C(O)N(CH₃)(CH₂CH═CH₂) |
| A-270 | N(H)C(O)N(C₂H₅)(CH₂CH═CH₂) |
| A-271 | N(H)C(O)NH(C₆H₅) |
| A-272 | N(H)C(O)N(CH₃)(C₆H₅) |
| A-273 | N(H)C(O)N(C₂H₅)(C₆H₅) |
| A-274 | N(CH₃)C(O)NH₂ |
| A-275 | N(CH₃)C(O)NH(CH₃) |
| A-276 | N(CH₃)C(O)NH(C₂H₅) |
| A-277 | N(CH₃)C(O)N(CH₃)₂ |
| A-278 | N(CH₃)C(O)N(CH₃)(C₂H₅) |
| A-279 | N(CH₃)C(O)N(C₂H₅)₂ |
| A-280 | N(CH₃)C(O)NH(CH₂CH₂CH₃) |
| A-281 | N(CH₃)C(O)N(CH₃)(CH₂CH₂CH₃) |
| A-282 | N(CH₃)C(O)N(C₂H₅)(CH₂CH₂CH₃) |
| A-283 | N(CH₃)C(O)NH(CH(CH₃)₂) |
| A-284 | N(CH₃)C(O)N(CH₃)(CH(CH₃)₂) |
| A-285 | N(CH₃)C(O)N(C₂H₅)(CH(CH₃)₂) |
| A-286 | N(CH₃)C(O)NH(CH(CH₃)(C₂H₅)) |
| A-287 | N(CH₃)C(O)N(CH₃)(CH(CH₃)(C₂H₅)) |
| A-288 | N(CH₃)C(O)N(C₂H₅)(CH(CH₃)(C₂H₅)) |
| A-289 | N(CH₃)C(O)NH(propargyl) |
| A-290 | N(CH₃)C(O)N(CH₃)(propargyl) |
| A-291 | N(CH₃)C(O)N(C₂H₅)(propargyl) |
| A-292 | N(CH₃)C(O)NH(CH₂CH═CH₂) |
| A-293 | N(CH₃)C(O)N(CH₃)(CH₂CH═CH₂) |
| A-294 | N(CH₃)C(O)N(C₂H₅)(CH₂CH═CH₂) |
| A-295 | N(CH₃)C(O)NH(C₆H₅) |
| A-296 | N(CH₃)C(O)N(CH₃)(C₆H₅) |
| A-297 | N(CH₃)C(O)N(C₂H₅)(C₆H₅) |
| A-298 | N(C₂H₅)C(O)NH₂ |
| A-299 | N(C₂H₅)C(O)NH(CH₃) |
| A-300 | N(C₂H₅)C(O)NH(C₂H₅) |
| A-301 | N(C₂H₅)C(O)N(CH₃)₂ |
| A-302 | N(C₂H₅)C(O)N(CH₃)(C₂H₅) |
| A-303 | N(C₂H₅)C(O)N(C₂H₅)₂ |
| A-304 | N(C₂H₅)C(O)NH(CH₂CH₂CH₃) |
| A-305 | N(C₂H₅)C(O)N(CH₃)(CH₂CH₂CH₃) |
| A-306 | N(C₂H₅)C(O)N(C₂H₅)(CH₂CH₂CH₃) |
| A-307 | N(C₂H₅)C(O)NH(CH(CH₃)₂) |
| A-308 | N(C₂H₅)C(O)N(CH₃)(CH(CH₃)₂) |
| A-309 | N(C₂H₅)C(O)N(C₂H₅)(CH(CH₃)₂) |
| A-310 | N(C₂H₅)C(O)NH(CH(CH₃)(C₂H₅)) |
| A-311 | N(C₂H₅)C(O)N(CH₃)(CH(CH₃)(C₂H₅)) |
| A-312 | N(C₂H₅)C(O)N(C₂H₅)(CH(CH₃)(C₂H₅)) |
| A-313 | N(C₂H₅)C(O)NH(propargyl) |
| A-314 | N(C₂H₅)C(O)N(CH₃)(propargyl) |
| A-315 | N(C₂H₅)C(O)N(C₂H₅)(propargyl) |
| A-316 | N(C₂H₅)C(O)NH(CH₂CH═CH₂) |
| A-317 | N(C₂H₅)C(O)N(CH₃)(CH₂CH═CH₂) |
| A-318 | N(C₂H₅)C(O)N(C₂H₅)(CH₂CH═CH₂) |
| A-319 | N(C₂H₅)C(O)NH(C₆H₅) |
| A-320 | N(C₂H₅)C(O)N(CH₃)(C₆H₅) |
| A-321 | N(C₂H₅)C(O)N(C₂H₅)(C₆H₅) |
| A-322 | N(propargyl)C(O)NH₂ |
| A-323 | N(propargyl)C(O)NH(CH₃) |
| A-324 | N(propargyl)C(O)NH(C₂H₅) |
| A-325 | N(propargyl)C(O)N(CH₃)₂ |
| A-326 | N(propargyl)C(O)N(CH₃)(C₂H₅) |
| A-327 | N(propargyl)C(O)N(C₂H₅)₂ |
| A-328 | N(propargyl)C(O)NH(CH₂CH₂CH₃) |
| A-329 | N(propargyl)C(O)N(CH₃)(CH₂CH₂CH₃) |
| A-330 | N(propargyl)C(O)N(C₂H₅)(CH₂CH₂CH₃) |
| A-331 | N(propargyl)C(O)NH(CH(CH₃)₂) |
| A-332 | N(propargyl)C(O)N(CH₃)(CH(CH₃)₂) |
| A-333 | N(propargyl)C(O)N(C₂H₅)(CH(CH₃)₂) |
| A-334 | N(propargyl)C(O)NH(CH(CH₃)(C₂H₅)) |
| A-335 | N(propargyl)C(O)N(CH₃)(CH(CH₃)(C₂H₅)) |
| A-336 | N(propargyl)C(O)N(C₂H₅)(CH(CH₃)(C₂H₅)) |
| A-337 | N(propargyl )C(O)NH(propargyl) |
| A-338 | N(propargyl)C(O)N(CH₃)(propargyl) |
| A-339 | N(propargyl)C(O)N(C₂H₅)(propargyl) |
| A-340 | N(propargyl)C(O)NH(CH₂CH═CH₂) |
| A-341 | N(propargyl)C(O)N(CH₃)(CH₂CH═CH₂) |
| A-342 | N(propargyl)C(O)N(C₂H₅)(CH₂CH═CH₂) |
| A-343 | N(propargyl)C(O)NH(C₆H₅) |
| A-344 | N(propargyl)C(O)N(CH₃)(C₆H₅) |
| A-345 | N(propargyl)C(O)N(C₂H₅)(C₆H₅) |
| A-346 | pyrrol-1-yl |
| A-347 | 2-methyl-pyrrol-1-yl |
| A-348 | 2,5-dimethyl-pyrrol-1-yl |
| A-349 | 2,4-dimethyl-3-ethylpyrrol-1-yl |
| A-350 | 3,4-dimethyl-pyrrol-1-yl |
| A-351 | 3-methyl-pyrrol-1-yl |
| A-352 | 3-chloro-pyrrol-1-yl |
| A-353 | 3-bromo-pyrrol-1-yl |
| A-354 | 3-acetoxymethyl-pyrrol-1-yl |
| A-355 | 3-hydroxymethyl-pyrrol-1-yl |
| A-356 | imidazol-1-yl |
| A-357 | pyrazol-1-yl |
| A-358 | [1,2,4]-triazol-1-yl |

propargyl—CH₂C≡CH
#point of attachment to S(O)ₘ

Table 1: Compounds of the formulae Ia, Ib and Ic in which R¹ is CH₃, R² is H, R³ and R⁴ are H and A for each individual compound corresponds in each case to one row of table A;
Table 2: Compounds of the formulae Ia, Ib and Ic in which R¹ is CH₃, R² is F, R³ and R⁴ are H and A for each individual compound corresponds in each case to one row of table A;
Table 3: Compounds of the formulae Ia, Ib and Ic in which R¹ is CH₃, R² is Cl, R³ and R⁴ are H and A for each individual compound corresponds in each case to one row of table A;
Table 4: Compounds of the formulae Ia, Ib and Ic in which R¹ is CH₃, R² is Br, R³ and R⁴ are H and A for each individual compound corresponds in each case to one row of table A;
Table 5: Compounds of the formulae Ia, Ib and Ic in which R¹ is CH₃, R² is I, R³ and R⁴ are H and A for each individual compound corresponds in each case to one row of table A;
Table 6: Compounds of the formulae Ia, Ib and Ic in which R¹ is CH₃, R² is CH₃, R³ and R⁴ are H and A for each individual compound corresponds in each case to one row of table A;
Table 7: Compounds of the formulae Ia, Ib and Ic in which R¹ is CH₃, R² is CF₃, R³ and R⁴ are H and A for each individual compound corresponds in each case to one row of table A;
Table 8: Compounds of the formulae Ia, Ib and Ic in which R¹ is CH₃, R² is CCl₃, R³ and R⁴ are H and A for each individual compound corresponds in each case to one row of table A;
Table 9: Compounds of the formulae Ia, Ib and Ic in which R¹ is CH₃, R³ is F, R² and R⁴ are H and A for each individual compound corresponds in each case to one row of table A;
Table 10: Compounds of the formulae Ia, Ib and Ic in which R¹ is CH₃, R³ is Cl, R² and R⁴ are H and A for each individual compound corresponds in each case to one row of table A;
Table 11: Compounds of the formulae Ia, Ib and Ic in which R¹ is CH₃, R³ is Br, R² and R⁴ are H and A for each individual compound corresponds in each case to one row of table A;
Table 12: Compounds of the formulae Ia, Ib and Ic in which R¹ is CH₃, R³ is I, R² and R⁴ are H and A for each individual compound corresponds in each case to one row of table A;
Table 13: Compounds of the formulae Ia, Ib and Ic in which R¹ is CH₃, R³ is CH₃, R² and R⁴ are H and A for each individual compound corresponds in each case to one row of table A;
Table 14: Compounds of the formulae Ia, Ib and Ic in which R¹ is CH₃, R³ is CF₃, R² and R⁴ are H and A for each individual compound corresponds in each case to one row of table A;
Table 15: Compounds of the formulae Ia, Ib and Ic in which R¹ is CH₃, R³ is CCl₃, R² and R⁴ are H and A for each individual compound corresponds in each case to one row of table A;
Table 16: Compounds of the formulae Ia, Ib and Ic in which R¹ is CH₃, R⁴ is F, R² and R³ are H and A for each individual compound corresponds in each case to one row of table A;
Table 17: Compounds of the formulae Ia, Ib and Ic in which R¹ is CH₃, R⁴ is Cl, R² and R³ are H and A for each individual compound corresponds in each case to one row of table A;
Table 18: Compounds of the formulae Ia, Ib and Ic in which R¹ is CH₃, R⁴ is Br, R² and R³ are H and A for each individual compound corresponds in each case to one row of table A;
Table 19: Compounds of the formulae Ia, Ib and Ic in which R¹ is CH₃, R⁴ is I, R² and R³ are H and A for each individual compound corresponds in each case to one row of table A;
Table 20: Compounds of the formulae Ia, Ib and Ic in which R¹ is CH₃, R⁴ is CH₃, R² and R³ are H and A for each individual compound corresponds in each case to one row of table A;
Table 21: Compounds of the formulae Ia, Ib and Ic in which R¹ is CH₃, R⁴ is CF₃, R² and R³ are H and A for each individual compound corresponds in each case to one row of table A;
Table 22: Compounds of the formulae Ia, Ib and Ic in which R¹ is CH₃, R⁴ is CCl₃, R² and R³ are H and A for each individual compound corresponds in each case to one row of table A;
Table 23: Compounds of the formulae Ia, Ib and Ic in which R¹ is C₂H₅, R² is H, R³ and R⁴ are H and A for each individual compound corresponds in each case to one row of table A;
Table 24: Compounds of the formulae Ia, Ib and Ic in which R¹ is C₂H₅, R² is F, R³ and R⁴ are H and A for each individual compound corresponds in each case to one row of table A;
Table 25: Compounds of the formulae Ia, Ib and Ic in which R¹ is C₂H₅, R² is Cl, R³ and R⁴ are H and A for each individual compound corresponds in each case to one row of table A;
Table 26: Compounds of the formulae Ia, Ib and Ic in which R¹ is C₂H₅, R² is Br, R³ and R⁴ are H and A for each individual compound corresponds in each case to one row of table A;
Table 27: Compounds of the formulae Ia, Ib and Ic in which R¹ is C₂H₅, R² is I, R³ and R⁴ are H and A for each individual compound corresponds in each case to one row of table A;
Table 28: Compounds of the formulae Ia, Ib and Ic in which R¹ is C₂H₅, R² is CH₃, R³ and R⁴ are H and A for each individual compound corresponds in each case to one row of table A;
Table 29: Compounds of the formulae Ia, Ib and Ic in which R¹ is C₂H₅, R² is CF₃, R³ and R⁴ are H and A for each individual compound corresponds in each case to one row of table A;
Table 30: Compounds of the formulae Ia, Ib and Ic in which R¹ is C₂H₅, R² is CCl₃, R³ and R⁴ are H and A for each individual compound corresponds in each case to one row of table A;
Table 31: Compounds of the formulae Ia, Ib and Ic in which R¹ is C₂H₅, R³ is F, R² and R⁴ are H and A for each individual compound corresponds in each case to one row of table A;
Table 32: Compounds of the formulae Ia, Ib and Ic in which R¹ is C₂H₅, R³ is Cl, R² and R⁴ are H and A for each individual compound corresponds in each case to one row of table A;
Table 33: Compounds of the formulae Ia, Ib and Ic in which R¹ is C₂H₅, R³ is Br, R² and R⁴ are H and A for each individual compound corresponds in each case to one row of table A;
Table 34: Compounds of the formulae Ia, Ib and Ic in which R¹ is C₂H₅, R³ is I, R² and R⁴ are H and A for each individual compound corresponds in each case to one row of table A;
Table 35: Compounds of the formulae Ia, Ib and Ic in which R¹ is C₂H₅, R³ is CH₃, R² and R⁴ are H and A for each individual compound corresponds in each case to one row of table A;
Table 36: Compounds of the formulae Ia, Ib and Ic in which R¹ is C₂H₅, R³ is CF₃, R² and R⁴ are H and A for each individual compound corresponds in each case to one row of table A;
Table 37: Compounds of the formulae Ia, Ib and Ic in which R¹ is C₂H₅, R³ is CCl₃, R² and R⁴ are H and A for each individual compound corresponds in each case to one row of table A;
Table 38: Compounds of the formulae Ia, Ib and Ic in which R¹ is C₂H₅, R⁴ is F, R² and R³ are H and A for each individual compound corresponds in each case to one row of table A;
Table 39: Compounds of the formulae Ia, Ib and Ic in which R¹ is C₂H₅, R⁴ is Cl, R² and R³ are H and A for each individual compound corresponds in each case to one row of table A;
Table 40: Compounds of the formulae Ia, Ib and Ic in which R¹ is C₂H₅, R⁴ is Br, R² and R³ are H and A for each individual compound corresponds in each case to one row of table A;
Table 41: Compounds of the formulae Ia, Ib and Ic in which R¹ is C₂H₅, R⁴ is 1, R² and R³ are H and A for each individual compound corresponds in each case to one row of table A;
Table 42: Compounds of the formulae Ia, Ib and Ic in which R¹ is C₂H₅, R⁴ is CH₃, R² and R³ are H and A for each individual compound corresponds in each case to one row of table A;
Table 43: Compounds of the formulae Ia, Ib and Ic in which R¹ is C₂H₅, R⁴ is CF₃, R² and R³ are H and A for each individual compound corresponds in each case to one row of table A;
Table 44: Compounds of the formulae Ia, Ib and Ic in which R¹ is C₂H₅, R⁴ is CCl₃, R² and R³ are H and A for each individual compound corresponds in each case to one row of table A;
Table 45: Compounds of the formulae Ia, Ib and Ic in which R¹ is F, R² is H, R³ and R⁴ are H and A for each individual compound corresponds in each case to one row of table A;
Table 46: Compounds of the formulae Ia, Ib and Ic in which R¹ is F, R² is F, R³ and R⁴ are H and A for each individual compound corresponds in each case to one row of table A;
Table 47: Compounds of the formulae Ia, Ib and Ic in which R¹ is F, R² is Cl, R³ and R⁴ are H and A for each individual compound corresponds in each case to one row of table A;
Table 48: Compounds of the formulae Ia, Ib and Ic in which R¹ is F, R² is Br, R³ and R⁴ are H and A for each individual compound corresponds in each case to one row of table A;
Table 49: Compounds of the formulae Ia, Ib and Ic in which R¹ is F, R² is I, R³ and R⁴ are H and A for each individual compound corresponds in each case to one row of table A;
Table 50: Compounds of the formulae Ia, Ib and Ic in which R¹ is F, R² is CH₃, R³ and R⁴ are H and A for each individual compound corresponds in each case to one row of table A;
Table 51: Compounds of the formulae Ia, Ib and Ic in which R¹ is F, R² is CF₃, R³ and R⁴ are H and A for each individual compound corresponds in each case to one row of table A;
Table 52: Compounds of the formulae Ia, Ib and Ic in which R¹ is F, R² is CCl₃, R³ and R⁴ are H and A for each individual compound corresponds in each case to one row of table A;
Table 53: Compounds of the formulae Ia, Ib and Ic in which R¹ is F, R³ is F, R² and R⁴ are H and A for each individual compound corresponds in each case to one row of table A;
Table 54: Compounds of the formulae Ia, Ib and Ic in which R¹ is F, R³ is Cl, R² and R⁴ are H and A for each individual compound corresponds in each case to one row of table A;
Table 55: Compounds of the formulae Ia, Ib and Ic in which R¹ is F, R³ is Br, R² and R⁴ are H and A for each individual compound corresponds in each case to one row of table A;
Table 56: Compounds of the formulae Ia, Ib and Ic in which R¹ is F, R³ is I, R² and R⁴ are H and A for each individual compound corresponds in each case to one row of table A;
Table 57: Compounds of the formulae Ia, Ib and Ic in which R¹ is F, R³ is CH₃, R² and R⁴ are H and A for each individual compound corresponds in each case to one row of table A;
Table 58: Compounds of the formulae Ia, Ib and Ic in which R¹ is F, R³ is CF₃, R² and R⁴ are H and A for each individual compound corresponds in each case to one row of table A;
Table 59: Compounds of the formulae Ia, Ib and Ic in which R¹ is F, R³ is CCl₃, R² and R⁴ are H and A for each individual compound corresponds in each case to one row of table A;
Table 60: Compounds of the formulae Ia, Ib and Ic in which R¹ is F, R⁴ is F, R² and R³ are H and A for each individual compound corresponds in each case to one row of table A;
Table 61: Compounds of the formulae Ia, Ib and Ic in which R¹ is F, R⁴ is Cl, R² and R³ are H and A for each individual compound corresponds in each case to one row of table A;
Table 62: Compounds of the formulae Ia, Ib and Ic in which R¹ is F, R⁴ is Br, R² and R³ are H and A for each individual compound corresponds in each case to one row of table A;
Table 63: Compounds of the formulae Ia, Ib and Ic in which R¹ is F, R⁴ is 1, R² and R³ are H and A for each individual compound corresponds in each case to one row of table A;
Table 64: Compounds of the formulae Ia, Ib and Ic in which R¹ is F, R⁴ is CH₃, R² and R³ are H and A for each individual compound corresponds in each case to one row of table A;
Table 65: Compounds of the formulae Ia, Ib and Ic in which R¹ is F, R⁴ is CF₃, R² and R³ are H and A for each individual compound corresponds in each case to one row of table A;
Table 66: Compounds of the formulae Ia, Ib and Ic in which R¹ is F, R⁴ is CCl₃, R² and R³ are H and A for each individual compound corresponds in each case to one row of table A;
Table 67: Compounds of the formulae Ia, Ib and Ic in which R¹ is Cl, R² is H, R³ and R⁴ are H and A for each individual compound corresponds in each case to one row of table A;
Table 68: Compounds of the formulae Ia, Ib and Ic in which R¹ is Cl, R² is F, R³ and R⁴ are H and A for each individual compound corresponds in each case to one row of table A;
Table 69: Compounds of the formulae Ia, Ib and Ic in which R¹ is Cl, R² is Cl, R³ and R⁴ are H and A for each individual compound corresponds in each case to one row of table A;
Table 70: Compounds of the formulae Ia, Ib and Ic in which R¹ is Cl, R² is Br, R³ and R⁴ are H and A for each individual compound corresponds in each case to one row of table A;
Table 71: Compounds of the formulae Ia, Ib and Ic in which R¹ is Cl, R² is 1, R³ and R⁴ are H and A for each individual compound corresponds in each case to one row of table A;
Table 72: Compounds of the formulae Ia, Ib and Ic in which R¹ is Cl, R² is CH₃, R³ and R⁴ are H and A for each individual compound corresponds in each case to one row of table A;
Table 73: Compounds of the formulae Ia, Ib and Ic in which R¹ is Cl, R² is CF₃, R³ and R⁴ are H and A for each individual compound corresponds in each case to one row of table A;
Table 74: Compounds of the formulae Ia, Ib and Ic in which R¹ is Cl, R² is CCl₃, R³ and R⁴ are H and A for each individual compound corresponds in each case to one row of table A;
Table 75: Compounds of the formulae Ia, Ib and Ic in which R¹ is Cl, R³ is F, R² and R⁴ are H and A for each individual compound corresponds in each case to one row of table A;
Table 76: Compounds of the formulae Ia, Ib and Ic in which R¹ is Cl, R³ is Cl, R² and R⁴ are H and A for each individual compound corresponds in each case to one row of table A;
Table 77: Compounds of the formulae Ia, Ib and Ic in which R¹ is Cl, R³ is Br, R² and R⁴ are H and A for each individual compound corresponds in each case to one row of table A;
Table 78: Compounds of the formulae Ia, Ib and Ic in which R¹ is Cl, R³ is I, R² and R⁴ are H and A for each individual compound corresponds in each case to one row of table A;
Table 79: Compounds of the formulae Ia, Ib and Ic in which R¹ is Cl, R³ is CH₃, R² and R⁴ are H and A for each individual compound corresponds in each case to one row of table A;
Table 80: Compounds of the formulae Ia, Ib and Ic in which R¹ is Cl, R³ is CF₃, R² and R⁴ are H and A for each individual compound corresponds in each case to one row of table A;
Table 81: Compounds of the formulae Ia, Ib and Ic in which R¹ is Cl, R³ is CCl₃, R² and R⁴ are H and A for each individual compound corresponds in each case to one row of table A;
Table 82: Compounds of the formulae Ia, Ib and Ic in which R¹ is Cl, R⁴ is F, R² and R³ are H and A for each individual compound corresponds in each case to one row of table A;
Table 83: Compounds of the formulae Ia, Ib and Ic in which R¹ is Cl, R⁴ is Cl, R² and R³ are H and A for each individual compound corresponds in each case to one row of table A;
Table 84: Compounds of the formulae Ia, Ib and Ic in which R¹ is Cl, R⁴ is Br, R² and R³ are H and A for each individual compound corresponds in each case to one row of table A;
Table 85: Compounds of the formulae Ia, Ib and Ic in which R¹ is Cl, R⁴ is 1, R² and R³ are H and A for each individual compound corresponds in each case to one row of table A;
Table 86: Compounds of the formulae Ia, Ib and Ic in which R¹ is Cl, R⁴ is CH₃, R² and R³ are H and A for each individual compound corresponds in each case to one row of table A;
Table 87: Compounds of the formulae Ia, Ib and Ic in which R¹ is Cl, R⁴ is CF₃, R² and R³ are H and A for each individual compound corresponds in each case to one row of table A;
Table 88: Compounds of the formulae Ia, Ib and Ic in which R¹ is Cl, R⁴ is CCl₃, R² and R³ are H and A for each individual compound corresponds in each case to one row of table A;
Table 89: Compounds of the formulae Ia, Ib and Ic in which R¹ is Br, R² is H, R³ and R⁴ are H and A for each individual compound corresponds in each case to one row of table A;
Table 90: Compounds of the formulae Ia, Ib and Ic in which R¹ is Br, R² is F, R³ and R⁴ are H and A for each individual compound corresponds in each case to one row of table A;
Table 91: Compounds of the formulae Ia, Ib and Ic in which R¹ is Br, R² is Cl, R³ and R⁴ are H and A for each individual compound corresponds in each case to one row of table A;
Table 92: Compounds of the formulae Ia, Ib and Ic in which R¹ is Br, R² is Br, R³ and R⁴ are H and A for each individual compound corresponds in each case to one row of table A;
Table 93: Compounds of the formulae Ia, Ib and Ic in which R¹ is Br, R² is 1, R³ and R⁴ are H and A for each individual compound corresponds in each case to one row of table A;
Table 94: Compounds of the formulae Ia, Ib and Ic in which R¹ is Br, R² is CH₃, R³ and R⁴ are H and A for each individual compound corresponds in each case to one row of table A;
Table 95: Compounds of the formulae Ia, Ib and Ic in which R¹ is Br, R² is CF₃, R³ and R⁴ are H and A for each individual compound corresponds in each case to one row of table A;
Table 96: Compounds of the formulae Ia, Ib and Ic in which R¹ is Br, R² is CCl₃, R³ and R⁴ are H and A for each individual compound corresponds in each case to one row of table A;
Table 97: Compounds of the formulae Ia, Ib and Ic in which R¹ is Br, R³ is F, R² and R⁴ are H and A for each individual compound corresponds in each case to one row of table A;
Table 98: Compounds of the formulae Ia, Ib and Ic in which R¹ is Br, R³ is Cl, R² and R⁴ are H and A for each individual compound corresponds in each case to one row of table A;
Table 99: Compounds of the formulae Ia, Ib and Ic in which R¹ is Br, R³ is Br, R² and R⁴ are H and A for each individual compound corresponds in each case to one row of table A;
Table 100: Compounds of the formulae Ia, Ib and Ic in which R¹ is Br, R³ is 1, R² and R⁴ are H and A for each individual compound corresponds in each case to one row of table A;
Table 101: Compounds of the formulae Ia, Ib and Ic in which R¹Br, R³ is CH₃, R² and R⁴ are H and A for each individual compound corresponds in each case to one row of table A;
Table 102: Compounds of the formulae Ia, Ib and Ic in which R¹ is CH₃, R³ is CF₃, R² and R⁴ are H and A for each individual compound corresponds in each case to one row of table A;
Table 103: Compounds of the formulae Ia, Ib and Ic in which R¹ is Br, R³ is CCl₃, R² and R⁴ are H and A for each individual compound corresponds in each case to one row of table A;
Table 104: Compounds of the formulae Ia, Ib and Ic in which R¹ is Br, R⁴ is F, R² and R³ are H and A for each individual compound corresponds in each case to one row of table A;
Table 105: Compounds of the formulae Ia, Ib and Ic in which R¹ is Br, R⁴ is Cl, R² and R³ are H and A for each individual compound corresponds in each case to one row of table A;
Table 106: Compounds of the formulae Ia, Ib and Ic in which R¹ is Br, R⁴ is Br, R² and R³ are H and A for each individual compound corresponds in each case to one row of table A;
Table 107: Compounds of the formulae Ia, Ib and Ic in which R¹ is Br, R⁴ is 1, R² and R³ are H and A for each individual compound corresponds in each case to one row of table A;
Table 108: Compounds of the formulae Ia, Ib and Ic in which R¹ is Br, R⁴ is CH₃, R² and R³ are H and A for each individual compound corresponds in each case to one row of table A;
Table 109: Compounds of the formulae Ia, Ib and Ic in which R¹ is Br, R⁴ is CF₃, R² and R³ are H and A for each individual compound corresponds in each case to one row of table A;
Table 110: Compounds of the formulae Ia, Ib and Ic in which R¹ is Br, R⁴ is CCl₃, R² and R³ are H and A for each individual compound corresponds in each case to one row of table A;
Table 111: Compounds of the formulae Ia, Ib and Ic in which R¹ is 1, R² is H, R³ and R⁴ are H and A for each individual compound corresponds in each case to one row of table A;
Table 112: Compounds of the formulae Ia, Ib and Ic in which R¹ is I, R² is F, R³ and R⁴ are H and A for each individual compound corresponds in each case to one row of table A;
Table 113: Compounds of the formulae Ia, Ib and Ic in which R¹ is 1, R² is Cl, R³ and R⁴ are H and A for each individual compound corresponds in each case to one row of table A;
Table 114: Compounds of the formulae Ia, Ib and Ic in which R¹ is 1, R² is Br, R³ and R⁴ are H and A for each individual compound corresponds in each case to one row of table A;
Table 115: Compounds of the formulae Ia, Ib and Ic in which R¹ is 1, R² is I, R³ and R⁴ are H and A for each individual compound corresponds in each case to one row of table A;
Table 116: Compounds of the formulae Ia, Ib and Ic in which R¹ is 1, R² is CH₃, R³ and R⁴ are H and A for each individual compound corresponds in each case to one row of table A;
Table 117: Compounds of the formulae Ia, Ib and Ic in which R¹ is 1, R² is CF₃, R³ and R⁴ are H and A for each individual compound corresponds in each case to one row of table A;
Table 118: Compounds of the formulae Ia, Ib and Ic in which R¹ is 1, R² is CCl₃, R³ and R⁴ are H and A for each individual compound corresponds in each case to one row of table A;
Table 119: Compounds of the formulae Ia, Ib and Ic in which R¹ is 1, R³ is F, R² and R⁴ are H and A for each individual compound corresponds in each case to one row of table A;
Table 120: Compounds of the formulae Ia, Ib and Ic in which R¹ is 1, R³ is Cl, R² and R⁴ are H and A for each individual compound corresponds in each case to one row of table A;
Table 121: Compounds of the formulae Ia, Ib and Ic in which R¹ is 1, R³ is Br, R² and R⁴ are H and A for each individual compound corresponds in each case to one row of table A;
Table 122: Compounds of the formulae Ia, Ib and Ic in which R¹ is 1, R³ is I, R² and R⁴ are H and A for each individual compound corresponds in each case to one row of table A;
Table 123: Compounds of the formulae Ia, Ib and Ic in which R¹ is 1, R³ is CH₃, R² and R⁴ are H and A for each individual compound corresponds in each case to one row of table A;
Table 124: Compounds of the formulae Ia, Ib and Ic in which R¹ is 1, R³ is CF₃, R² and R⁴ are H and A for each individual compound corresponds in each case to one row of table A;
Table 125: Compounds of the formulae Ia, Ib and Ic in which R¹ is 1, R³ is CCl₃, R² and R⁴ are H and A for each individual compound corresponds in each case to one row of table A;
Table 126: Compounds of the formulae Ia, Ib and Ic in which R¹ is 1, R⁴ is F, R² and R³ are H and A for each individual compound corresponds in each case to one row of table A;
Table 127: Compounds of the formulae Ia, Ib and Ic in which R¹ is 1, R⁴ is Cl, R² and R³ are H and A for each individual compound corresponds in each case to one row of table A;
Table 128: Compounds of the formulae Ia, Ib and Ic in which R¹ is 1, R⁴ is Br, R² and R³ are H and A for each individual compound corresponds in each case to one row of table A;
Table 129: Compounds of the formulae Ia, Ib and Ic in which R¹ is 1, R⁴ is 1, R² and R³ are H and A for each individual compound corresponds in each case to one row of table A;
Table 130: Compounds of the formulae Ia, Ib and Ic in which R¹ is 1, R⁴ is CH₃, R² and R³ are H and A for each individual compound corresponds in each case to one row of table A;
Table 131: Compounds of the formulae Ia, Ib and Ic in which R¹ is 1, R⁴ is CF₃, R² and R³ are H and A for each individual compound corresponds in each case to one row of table A;
Table 132: Compounds of the formulae Ia, Ib and Ic in which R¹ is 1, R⁴ is CCl₃, R² and R³ are H and A for each individual compound corresponds in each case to one row of table A;
Table 133: Compounds of the formulae Ia, Ib and Ic in which R¹ is CF₃, R² is H, R³ and R⁴ are H and A for each individual compound corresponds in each case to one row of table A;
Table 134: Compounds of the formulae Ia, Ib and Ic in which R¹ is CF₃, R² is F, R³ and R⁴ are H and A for each individual compound corresponds in each case to one row of table A;
Table 135: Compounds of the formulae Ia, Ib and Ic in which R¹ is CF₃, R² is Cl, R³ and R⁴ are H and A for each individual compound corresponds in each case to one row of table A;
Table 136: Compounds of the formulae Ia, Ib and Ic in which R¹ is CF₃, R² is Br, R³ and R⁴ are H and A for each individual compound corresponds in each case to one row of table A;
Table 137: Compounds of the formulae Ia, Ib and Ic in which R¹ is CF₃, R² is I, R³ and R⁴ are H and A for each individual compound corresponds in each case to one row of table A;
Table 138: Compounds of the formulae Ia, Ib and Ic in which R¹ is CF₃, R² is CH₃, R³ and R⁴ are H and A for each individual compound corresponds in each case to one row of table A;
Table 139: Compounds of the formulae Ia, Ib and Ic in which R¹ is CF₃, R² is CF₃, R³ and R⁴ are H and A for each individual compound corresponds in each case to one row of table A;
Table 140: Compounds of the formulae Ia, Ib and Ic in which R¹ is CF₃, R² is CCl₃, R³ and R⁴ are H and A for each individual compound corresponds in each case to one row of table A;
Table 141: Compounds of the formulae Ia, Ib and Ic in which R¹ is CF₃, R³ is F, R² and R⁴ are H and A for each individual compound corresponds in each case to one row of table A;
Table 142: Compounds of the formulae Ia, Ib and Ic in which R¹ is CF₃, R³ is Cl, R² and R⁴ are H and A for each individual compound corresponds in each case to one row of table A;
Table 143: Compounds of the formulae Ia, Ib and Ic in which R¹ is CF₃, R³ is Br, R² and R⁴ are H and A for each individual compound corresponds in each case to one row of table A;
Table 144: Compounds of the formulae Ia, Ib and Ic in which R¹ is CF₃, R³ is I, R² and R⁴ are H and A for each individual compound corresponds in each case to one row of table A;
Table 145: Compounds of the formulae Ia, Ib and Ic in which R¹ is CF₃, R³ is CH₃, R² and R⁴ are H and A for each individual compound corresponds in each case to one row of table A;
Table 146: Compounds of the formulae Ia, Ib and Ic in which R¹ is CF₃, R³ is CF₃, R² and R⁴ are H and A for each individual compound corresponds in each case to one row of table A;
Table 147: Compounds of the formulae Ia, Ib and Ic in which R¹ is CF₃, R³ is CCl₃, R² and R⁴ are H and A for each individual compound corresponds in each case to one row of table A;
Table 148: Compounds of the formulae Ia, Ib and Ic in which R¹ is CF₃, R⁴ is F, R² and R³ are H and A for each individual compound corresponds in each case to one row of table A;
Table 149: Compounds of the formulae Ia, Ib and Ic in which R¹ is CF₃, R⁴ is Cl, R² and R³ are H and A for each individual compound corresponds in each case to one row of table A;
Table 150: Compounds of the formulae Ia, Ib and Ic in which R¹ is CF₃, R⁴ is Br, R² and R³ are H and A for each individual compound corresponds in each case to one row of table A;
Table 151: Compounds of the formulae Ia, Ib and Ic in which R¹ is CF₃, R⁴ is 1, R² and R³ are H and A for each individual compound corresponds in each case to one row of table A;
Table 152: Compounds of the formulae Ia, Ib and Ic in which R¹ is CF₃, R⁴ is CH₃, R² and R³ are H and A for each individual compound corresponds in each case to one row of table A;
Table 153: Compounds of the formulae Ia, Ib and Ic in which R¹ is CF₃, R⁴ is CF₃, R² and R³ are H and A for each individual compound corresponds in each case to one row of table A;
Table 154: Compounds of the formulae Ia, Ib and Ic in which R¹ is CF₃, R⁴ is CCl₃, R² and R³ are H and A for each individual compound corresponds in each case to one row of table A;
Table 155: Compounds of the formulae Ia, Ib and Ic in which R¹ is OCH₃, R² is H, R³ and R⁴ are H and A for each individual compound corresponds in each case to one row of table A;
Table 156: Compounds of the formulae Ia, Ib and Ic in which R¹ is OCH₃, R² is F, R³ and R⁴ are H and A for each individual compound corresponds in each case to one row of table A;
Table 157: Compounds of the formulae Ia, Ib and Ic in which R¹ is OCH₃, R² is Cl, R³ and R⁴ are H and A for each individual compound corresponds in each case to one row of table A;
Table 158: Compounds of the formulae Ia, Ib and Ic in which R¹ is OCH₃, R² is Br, R³ and R⁴ are H and A for each individual compound corresponds in each case to one row of table A;
Table 159: Compounds of the formulae Ia, Ib and Ic in which R¹ is OCH₃, R² is I, R³ and R⁴ are H and A for each individual compound corresponds in each case to one row of table A;
Table 160: Compounds of the formulae Ia, Ib and Ic in which R¹ is OCH₃, R² is CH₃, R³ and R⁴ are H and A for each individual compound corresponds in each case to one row of table A;
Table 161: Compounds of the formulae Ia, Ib and Ic in which R¹ is OCH₃, R² is CF₃, R³ and R⁴ are H and A for each individual compound corresponds in each case to one row of table A;
Table 162: Compounds of the formulae Ia, Ib and Ic in which R¹ is OCH₃, R² is CCl₃, R³ and R⁴ are H and A for each individual compound corresponds in each case to one row of table A;
Table 163: Compounds of the formulae Ia, Ib and Ic in which R¹ is OCH₃, R³ is F, R² and R⁴ are H and A for each individual compound corresponds in each case to one row of table A;
Table 164: Compounds of the formulae Ia, Ib and Ic in which R¹ is OCH₃, R³ is Cl, R² and R⁴ are H and A for each individual compound corresponds in each case to one row of table A;
Table 165: Compounds of the formulae Ia, Ib and Ic in which R¹ is OCH₃, R³ is Br, R² and R⁴ are H and A for each individual compound corresponds in each case to one row of table A;
Table 166: Compounds of the formulae Ia, Ib and Ic in which R¹ is OCH₃, R³ is I, R² and R⁴ are H and A for each individual compound corresponds in each case to one row of table A;
Table 167: Compounds of the formulae Ia, Ib and Ic in which R¹ is OCH₃, R³ is CH₃, R² and R⁴ are H and A for each individual compound corresponds in each case to one row of table A;
Table 168: Compounds of the formulae Ia, Ib and Ic in which R¹ is OCH₃, R³ is CF₃, R² and R⁴ are H and A for each individual compound corresponds in each case to one row of table A;
Table 169: Compounds of the formulae Ia, Ib and Ic in which R¹ is OCH₃, R³ is CCl₃, R² and R⁴ are H and A for each individual compound corresponds in each case to one row of table A;
Table 170: Compounds of the formulae Ia, Ib and Ic in which R¹ is OCH₃, R⁴ is F, R² and R³ are H and A for each individual compound corresponds in each case to one row of table A;
Table 171: Compounds of the formulae Ia, Ib and Ic in which R¹ is OCH₃, R⁴ is Cl, R² and R³ are H and A for each individual compound corresponds in each case to one row of table A;
Table 172: Compounds of the formulae Ia, Ib and Ic in which R¹ is OCH₃, R⁴ is Br, R² and R³ are H and A for each individual compound corresponds in each case to one row of table A;
Table 173: Compounds of the formulae Ia, Ib and Ic in which R¹ is OCH₃, R⁴ is I, R² and R³ are H and A for each individual compound corresponds in each case to one row of table A;
Table 174: Compounds of the formulae Ia, Ib and Ic in which R¹ is OCH₃, R⁴ is CH₃, R² and R³ are H and A for each individual compound corresponds in each case to one row of table A;
Table 175: Compounds of the formulae Ia, Ib and Ic in which R¹ is OCH₃, R⁴ is CF₃, R² and R³ are H and A for each individual compound corresponds in each case to one row of table A;
Table 176: Compounds of the formulae Ia, Ib and Ic in which R¹ is OCH₃, R⁴ is CCl₃, R² and R³ are H and A for each individual compound corresponds in each case to one row of table A;
Table 177: Compounds of the formulae Ia, Ib and Ic in which R¹ is OC₂H₅, R² is H, R³ and R⁴ are H and A for each individual compound corresponds in each case to one row of table A;
Table 178: Compounds of the formulae Ia, Ib and Ic in which R¹ is OC₂H₅, R² is F, R³ and R⁴ are H and A for each individual compound corresponds in each case to one row of table A;
Table 179: Compounds of the formulae Ia, Ib and Ic in which R¹ is OC₂H₅, R² is Cl, R³ and R⁴ are H and A for each individual compound corresponds in each case to one row of table A;
Table 180: Compounds of the formulae Ia, Ib and Ic in which R¹ is OC₂H₅, R² is Br, R³ and R⁴ are H and A for each individual compound corresponds in each case to one row of table A;
Table 181: Compounds of the formulae Ia, Ib and Ic in which R¹ is OC₂H₅, R² is I, R³ and R⁴ are H and A for each individual compound corresponds in each case to one row of table A;
Table 182: Compounds of the formulae Ia, Ib and Ic in which R¹ is OC₂H₅, R² is CH₃, R³ and R⁴ are H and A for each individual compound corresponds in each case to one row of table A;
Table 183: Compounds of the formulae Ia, Ib and Ic in which R¹ is OC₂H₅, R² is CF₃, R³ and R⁴ are H and A for each individual compound corresponds in each case to one row of table A;
Table 184: Compounds of the formulae Ia, Ib and Ic in which R¹ is OC₂H₅, R² is CCl₃, R³ and R⁴ are H and A for each individual compound corresponds in each case to one row of table A;
Table 185: Compounds of the formulae Ia, Ib and Ic in which R¹ is OC₂H₅, R³ is F, R² and R⁴ are H and A for each individual compound corresponds in each case to one row of table A;
Table 186: Compounds of the formulae Ia, Ib and Ic in which R¹ is OC₂H₅, R³ is Cl, R² and R⁴ are H and A for each individual compound corresponds in each case to one row of table A;
Table 187: Compounds of the formulae Ia, Ib and Ic in which R¹ is OC₂H₅, R³ is Br, R² and R⁴ are H and A for each individual compound corresponds in each case to one row of table A;
Table 188: Compounds of the formulae Ia, Ib and Ic in which R¹ is OC₂H₅, R³ is I, R² and R⁴ are H and A for each individual compound corresponds in each case to one row of table A;
Table 189: Compounds of the formulae Ia, Ib and Ic in which R¹ is OC₂H₅, R³ is CH₃, R² and R⁴ are H and A for each individual compound corresponds in each case to one row of table A;
Table 190: Compounds of the formulae Ia, Ib and Ic in which R¹ is OC₂H₅, R³ is CF₃, R² and R⁴ are H and A for each individual compound corresponds in each case to one row of table A;
Table 191: Compounds of the formulae Ia, Ib and Ic in which R¹ is OC₂H₅, R³ is CCl₃, R² and R⁴ are H and A for each individual compound corresponds in each case to one row of table A;
Table 192: Compounds of the formulae Ia, Ib and Ic in which R¹ is OC₂H₅, R⁴ is F, R² and R³ are H and A for each individual compound corresponds in each case to one row of table A;
Table 193: Compounds of the formulae Ia, Ib and Ic in which R¹ is OC₂H₅, R⁴ is Cl, R² and R³ are H and A for each individual compound corresponds in each case to one row of table A;
Table 194: Compounds of the formulae Ia, Ib and Ic in which R¹ is OC₂H₅, R⁴ is Br, R² and R³ are H and A for each individual compound corresponds in each case to one row of table A;
Table 195: Compounds of the formulae Ia, Ib and Ic in which R¹ is OC₂H₅, R⁴ is 1, R² and R³ are H and A for each individual compound corresponds in each case to one row of table A;
Table 196: Compounds of the formulae Ia, Ib and Ic in which R¹ is OC₂H₅, R⁴ is CH₃, R² and R³ are H and A for each individual compound corresponds in each case to one row of table A;
Table 197: Compounds of the formulae Ia, Ib and Ic in which R¹ is OC₂H₅, R⁴ is CF₃, R² and R³ are H and A for each individual compound corresponds in each case to one row of table A;
Table 198: Compounds of the formulae Ia, Ib and Ic in which R¹ is OC₂H₅, R⁴ is CCl₃, R² and R³ are H and A for each individual compound corresponds in each case to one row of table A;
Table 199: Compounds of the formulae Ia, Ib and Ic in which R¹ is OCF₃, R² is H, R³ and R⁴ are H and A for each individual compound corresponds in each case to one row of table A;
Table 200: Compounds of the formulae Ia, Ib and Ic in which R¹ is OCF₃, R² is F, R³ and R⁴ are H and A for each individual compound corresponds in each case to one row of table A;
Table 201: Compounds of the formulae Ia, Ib and Ic in which R¹ is OCF₃, R² is Cl, R³ and R⁴ are H and A for each individual compound corresponds in each case to one row of table A;
Table 202: Compounds of the formulae Ia, Ib and Ic in which R¹ is OCF₃, R² is Br, R³ and R⁴ are H and A for each individual compound corresponds in each case to one row of table A;
Table 203: Compounds of the formulae Ia, Ib and Ic in which R¹ is OCF₃, R² is I, R³ and R⁴ are H and A for each individual compound corresponds in each case to one row of table A;
Table 204: Compounds of the formulae Ia, Ib and Ic in which R¹ is OCF₃, R² is CH₃, R³ and R⁴ are H and A for each individual compound corresponds in each case to one row of table A;
Table 205: Compounds of the formulae Ia, Ib and Ic in which R¹ is OCF₃, R² is CF₃, R³ and R⁴ are H and A for each individual compound corresponds in each case to one row of table A;
Table 206: Compounds of the formulae Ia, Ib and Ic in which R¹ is OCF₃, R² is CCl₃, R³ and R⁴ are H and A for each individual compound corresponds in each case to one row of table A;
Table 207: Compounds of the formulae Ia, Ib and Ic in which R¹ is OCF₃, R³ is F, R² and R⁴ are H and A for each individual compound corresponds in each case to one row of table A;
Table 208: Compounds of the formulae Ia, Ib and Ic in which R¹ is OCF₃, R³ is Cl, R² and R⁴ are H and A for each individual compound corresponds in each case to one row of table A;
Table 209: Compounds of the formulae Ia, Ib and Ic in which R¹ is OCF₃, R³ is Br, R² and R⁴ are H and A for each individual compound corresponds in each case to one row of table A;
Table 210: Compounds of the formulae Ia, Ib and Ic in which R¹ is OCF₃, R³ is I, R² and R⁴ are H and A for each individual compound corresponds in each case to one row of table A;
Table 211: Compounds of the formulae Ia, Ib and Ic in which R¹ is OCF₃, R³ is CH₃, R² and R⁴ are H and A for each individual compound corresponds in each case to one row of table A;
Table 212: Compounds of the formulae Ia, Ib and Ic in which R¹ is OCF₃, R³ is CF₃, R² and R⁴ are H and A for each individual compound corresponds in each case to one row of table A;
Table 213: Compounds of the formulae Ia, Ib and Ic in which R¹ is OCF₃, R³ is CCl₃, R² and R⁴ are H and A for each individual compound corresponds in each case to one row of table A;
Table 214: Compounds of the formulae Ia, Ib and Ic in which R¹ is OCF₃, R⁴ is F, R² and R³ are H and A for each individual compound corresponds in each case to one row of table A;
Table 215: Compounds of the formulae Ia, Ib and Ic in which R¹ is OCF₃, R⁴ is Cl, R² and R³ are H and A for each individual compound corresponds in each case to one row of table A;
Table 216: Compounds of the formulae Ia, Ib and Ic in which R¹ is OCF₃, R⁴ is Br, R² and R³ are H and A for each individual compound corresponds in each case to one row of table A;
Table 217: Compounds of the formulae Ia, Ib and Ic in which R¹ is OCF₃, R⁴ is 1, R² and R³ are H and A for each individual compound corresponds in each case to one row of table A;
Table 218: Compounds of the formulae Ia, Ib and Ic in which R¹ is OCF₃, R⁴ is CH₃, R² and R³ are H and A for each individual compound corresponds in each case to one row of table A;
Table 219: Compounds of the formulae Ia, Ib and Ic in which R¹ is OCF₃, R⁴ is CF₃, R² and R³ are H and A for each individual compound corresponds in each case to one row of table A;
Table 220: Compounds of the formulae Ia, Ib and Ic in which R¹ is OCF₃, R⁴ is CCl₃, R² and R³ are H and A for each individual compound corresponds in each case to one row of table A;
Table 221: Compounds of the formulae Ia, Ib and Ic in which R¹ is OCHF₂, R² is H, R³ and R⁴ are H and A for each individual compound corresponds in each case to one row of table A;
Table 222: Compounds of the formulae Ia, Ib and Ic in which R¹ is OCHF₂, R² is F, R³ and R⁴ are H and A for each individual compound corresponds in each case to one row of table A;
Table 223: Compounds of the formulae Ia, Ib and Ic in which R¹ is OCHF₂, R² is Cl, R³ and R⁴ are H and A for each individual compound corresponds in each case to one row of table A;
Table 224: Compounds of the formulae Ia, Ib and Ic in which R¹ is OCHF₂, R² is Br, R³ and R⁴ are H and A for each individual compound corresponds in each case to one row of table A;
Table 225: Compounds of the formulae Ia, Ib and Ic in which R¹ is OCHF₂, R² is I, R³ and R⁴ are H and A for each individual compound corresponds in each case to one row of table A;
Table 226: Compounds of the formulae Ia, Ib and Ic in which R¹ is OCHF₂, R² is CH₃, R³ and R⁴ are H and A for each individual compound corresponds in each case to one row of table A;
Table 227: Compounds of the formulae Ia, Ib and Ic in which R¹ is OCHF₂, R² is CF₃, R³ and R⁴ are H and A for each individual compound corresponds in each case to one row of table A;
Table 228: Compounds of the formulae Ia, Ib and Ic in which R¹ is OCHF₂, R² is CCl₃, R³ and R⁴ are H and A for each individual compound corresponds in each case to one row of table A;
Table 229: Compounds of the formulae Ia, Ib and Ic in which R¹ is OCHF₂, R³ is F, R² and R⁴ are H and A for each individual compound corresponds in each case to one row of table A;
Table 230: Compounds of the formulae Ia, Ib and Ic in which R¹ is OCHF₂, R³ is Cl, R² and R⁴ are H and A for each individual compound corresponds in each case to one row of table A;
Table 231: Compounds of the formulae Ia, Ib and Ic in which R¹ is OCHF₂, R³ is Br, R² and R⁴ are H and A for each individual compound corresponds in each case to one row of table A;
Table 232: Compounds of the formulae Ia, Ib and Ic in which R¹ is OCHF₂, R³ is I, R² and R⁴ are H and A for each individual compound corresponds in each case to one row of table A;
Table 233: Compounds of the formulae Ia, Ib and Ic in which R¹ is OCHF₂, R³ is CH₃, R² and R⁴ are H and A for each individual compound corresponds in each case to one row of table A;
Table 234: Compounds of the formulae Ia, Ib and Ic in which R¹ is OCHF₂, R³ is CF₃, R² and R⁴ are H and A for each individual compound corresponds in each case to one row of table A;
Table 235: Compounds of the formulae Ia, Ib and Ic in which R¹ is OCHF₂, R³ is CCl₃, R² and R⁴ are H and A for each individual compound corresponds in each case to one row of table A;
Table 236: Compounds of the formulae Ia, Ib and Ic in which R¹ is OCHF₂, R⁴ is F, R² and R³ are H and A for each individual compound corresponds in each case to one row of table A;
Table 237: Compounds of the formulae Ia, Ib and Ic in which R¹ is OCHF₂, R⁴ is Cl, R² and R³ are H and A for each individual compound corresponds in each case to one row of table A;
Table 238: Compounds of the formulae Ia, Ib and Ic in which R¹ is OCHF₂, R⁴ is Br, R² and R³ are H and A for each individual compound corresponds in each case to one row of table A;
Table 239: Compounds of the formulae Ia, Ib and Ic in which R¹ is OCHF₂, R⁴ is I, R² and R³ are H and A for each individual compound corresponds in each case to one row of table A;
Table 240: Compounds of the formulae Ia, Ib and Ic in which R¹ is OCHF₂, R⁴ is CH₃, R² and R³ are H and A for each individual compound corresponds in each case to one row of table A;
Table 241: Compounds of the formulae Ia, Ib and Ic in which R¹ is OCHF₂, R⁴ is CF₃, R² and R³ are H and A for each individual compound corresponds in each case to one row of table A;
Table 242: Compounds of the formulae Ia, Ib and Ic in which R¹ is OCHF₂, R⁴ is CCl₃, R² and R³ are H and A for each individual compound corresponds in each case to one row of table A;
Table 243: Compounds of the formulae Ia, Ib and Ic in which R¹ is OCClF₂, R² is H, R³ and R⁴ are H and A for each individual compound corresponds in each case to one row of table A;
Table 244: Compounds of the formulae Ia, Ib and Ic in which R¹ is OCClF₂, R² is F, R³ and R⁴ are H and A for each individual compound corresponds in each case to one row of table A;
Table 245: Compounds of the formulae Ia, Ib and Ic in which R¹ is OCClF₂, R² is Cl, R³ and R⁴ are H and A for each individual compound corresponds in each case to one row of table A;
Table 246: Compounds of the formulae Ia, Ib and Ic in which R¹ is OCClF₂, R² is Br, R³ and R⁴ are H and A for each individual compound corresponds in each case to one row of table A;
Table 247: Compounds of the formulae Ia, Ib and Ic in which R¹ is OCClF₂, R² is I, R³ and R⁴ are H and A for each individual compound corresponds in each case to one row of table A;
Table 248: Compounds of the formulae Ia, Ib and Ic in which R¹ is OCClF₂, R² is CH₃, R³ and R⁴ are H and A for each individual compound corresponds in each case to one row of table A;
Table 249: Compounds of the formulae Ia, Ib and Ic in which R¹ is OCClF₂, R² is CF₃, R³ and R⁴ are H and A for each individual compound corresponds in each case to one row of table A;
Table 250: Compounds of the formulae Ia, Ib and Ic in which R¹ is OCClF₂, R² is CCl₃, R³ and R⁴ are H and A for each individual compound corresponds in each case to one row of table A;
Table 251: Compounds of the formulae Ia, Ib and Ic in which R¹ is OCClF₂, R³ is F, R² and R⁴ are H and A for each individual compound corresponds in each case to one row of table A;
Table 252: Compounds of the formulae Ia, Ib and Ic in which R¹ is OCClF₂, R³ is Cl, R² and R⁴ are H and A for each individual compound corresponds in each case to one row of table A;
Table 253: Compounds of the formulae Ia, Ib and Ic in which R¹ is OCClF₂, R³ is Br, R² and R⁴ are H and A for each individual compound corresponds in each case to one row of table A;
Table 254: Compounds of the formulae Ia, Ib and Ic in which R¹ is OCClF₂, R³ is I, R² and R⁴ are H and A for each individual compound corresponds in each case to one row of table A;
Table 255: Compounds of the formulae Ia, Ib and Ic in which R¹ is OCClF₂, R³ is CH₃, R² and R⁴ are H and A for each individual compound corresponds in each case to one row of table A;
Table 256: Compounds of the formulae Ia, Ib and Ic in which R¹ is OCClF₂, R³ is CF₃, R² and R⁴ are H and A for each individual compound corresponds in each case to one row of table A;
Table 257: Compounds of the formulae Ia, Ib and Ic in which R¹ is OCClF₂, R³ is CCl₃, R² and R⁴ are H and A for each individual compound corresponds in each case to one row of table A;
Table 258: Compounds of the formulae Ia, Ib and Ic in which R¹ is OCClF₂, R⁴ is F, R² and R³ are H and A for each individual compound corresponds in each case to one row of table A;
Table 259: Compounds of the formulae Ia, Ib and Ic in which R¹ is OCClF₂, R⁴ is Cl, R² and R³ are H and A for each individual compound corresponds in each case to one row of table A;
Table 260: Compounds of the formulae Ia, Ib and Ic in which R¹ is OCClF₂, R⁴ is Br, R² and R³ are H and A for each individual compound corresponds in each case to one row of table A;
Table 261: Compounds of the formulae Ia, Ib and Ic in which R¹ is OCClF₂, R⁴ is I, R² and R³ are H and A for each individual compound corresponds in each case to one row of table A;
Table 262: Compounds of the formulae Ia, Ib and Ic in which R¹ is OCClF₂, R⁴ is CH₃, R² and R³ are H and A for each individual compound corresponds in each case to one row of table A;
Table 263: Compounds of the formulae Ia, Ib and Ic in which R¹ is OCClF₂, R⁴ is CF₃, R² and R³ are H and A for each individual compound corresponds in each case to one row of table A;
Table 264: Compounds of the formulae Ia, Ib and Ic in which R¹ is OCClF₂, R⁴ is CCl₃, R² and R³ are H and A for each individual compound corresponds in each case to one row of table A.

2-Cyanobenzene compounds Ia, in which A is a radical of the formula NR¹⁰C(O)—R⁹ in which R⁹ is aryl, aryl-C₁-C₄-alkyl, heteroaryl, heteroaryl-C₁-C₄-alkyl, heterocyclyl, heterocyclyl-C₁-C₄-alkyl, C₁-C₁₀-alkyl or C₃-C₁₀-cycloalkyl and R¹⁰ has the meanings given above may be prepared, for example, by reacting a 2-cyanobenzene sulfonamide (II) with an acyl halide (IIIa), especially an acyl chloride, an acid anhydride (IIIb) or a mixed acid anhydride in the presence of an appropriate base, see scheme 1. Appropriate bases include organic bases, for example tertiary amines, such as aliphatic amines, for example trimethylamine, triethylamine or diisopropylethylamine, cycloaliphatic tertiary amines, for example N-methylpiperidine, or aromatic amines, for example pyridine, substituted pyridines such as 2,4,6-collidine, 2,4-lutidine or 4-dimethylaminopyridine. The reaction is usually carried out in a solvent, for example an alkyl cyanide, such as acetonitrile or propionitrile. The compounds of the formula Ia can be prepared analogously to a procedure described in Bull. Chem. Soc. Jpn. 1988, 61, 3999-4004.

The reaction may also be carried out under acidic conditions, e.g. with sulfuric acid as catalyst. Usually the reaction is carried out in a suitable solvent. Suitable solvents are polar and inert under the given reaction conditions. Examples of suitable solvents include alkyl cyanides such as acetonitrile or propionitrile. The reaction is e.g. analogous to a procedure described in Tetrahedron Letters 2003, 44, 5461-5463 to which reference is made

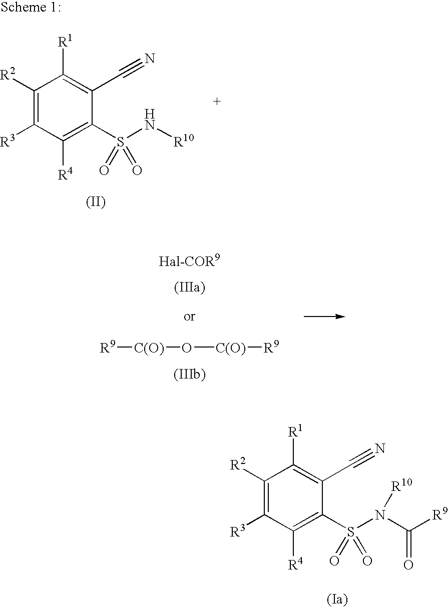

In scheme 1, the variables R¹, R², R³, R⁴ have the meanings mentioned above and in particular the meanings mentioned as being preferred and Hal is halogen, especially chlorine.

2-Cyanobenzene compounds Ia, in which A is a radical of the formula NR¹⁰C(O)—R⁹, wherein R⁹ is OR^{9a} in which R^{9a} has the meanings mentioned above and R¹⁰ has the meanings mentioned above, may be prepared by reacting a 2-cyanobenzene sulfonamide (II) with chloroformates of the formula CIC(O)OR^{9a} in the presence of suitable bases. Suitable bases include those mentioned above, e.g., triethylamine. The reaction is usually carried out in an appropriate solvent such as an alkyl cyanide, for example acetonitrile or propionitrile. The reaction can be carried out analogously to processes known from the prior art, for example in accordance with J. Med. Chem. 2004, 47, 627-5643.

2-Cyanobenzene compounds Ia, in which A is a radical of the formula NR¹⁰—C(O)—R⁹, wherein R⁹ and R¹⁰ together with the adjacent nitrogen and carbon atoms form a saturated or ethylenically unsaturated 5- to 10-membered ring as defined above and which may be substituted as described above, may be obtained by reacting a sulfonylhalide (IV), especially a sulfonylchloride, with a cyclic amide (V) in the presence of a strong base such as an organometallic base, for example an alkyllithium compound or aryllithium compound, e.g. n-butyllithium, tert-butyllithium or phenyllithium. The reaction is usually carried out in a polar organic solvent which is inert under the reaction conditions such as ether, for example, dialkyl ether or cyclic ethers such as tetrahydrofuran (THF) or dioxane, see scheme 2. In scheme 2, the variables R¹, R², R³, R⁴ have the meanings mentioned above and in particular the meanings mentioned as being preferred and Hal is halogen, especially chlorine.

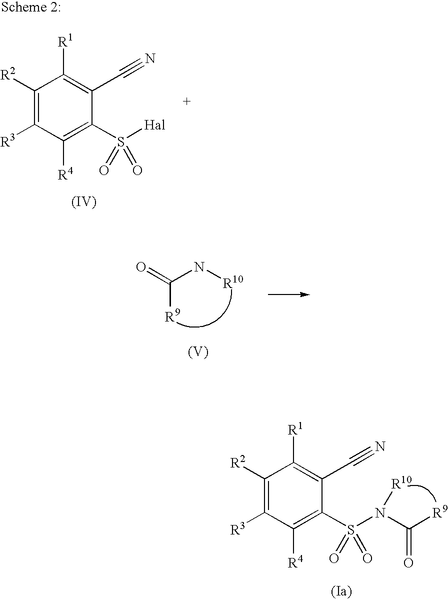

2-Cyanobenzene compounds Ia, in which A is a radical NR¹⁰—C(O)—R⁹, wherein R⁹ is a radical NR^{9b}R^{9c} in which R^{9b} and R^{9c} are as defined above and R¹⁰ has the meanings given above may be obtained by reacting a 2-cyanobenzene sulfonamide (II) with an appropriately substituted isocyanate (VI). The reaction can be performed in analogy to a process described in J. Med. Chem. 1979, 22, 321-325 (see scheme 3). In scheme 3, the variables R¹, R², R³, R⁴ have the meanings mentioned above and in particular the meanings mentioned as being preferred.

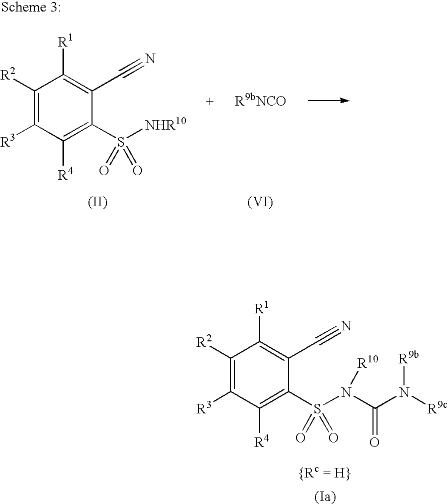

Conversion of compounds Ia in which R^{9c} is H to compounds Ia in which R^{9c} is different from H may be obtained by alkylating the compounds Ia (where R^{9c} is H and which are obtainable according to scheme 3) with suitable alkylating agents in the presence of a base. The required reaction conditions are known in the art, for example from J. March, Advanced Organic Synthesis, 3^{nd} edition, Wiley Interscience, New York, 1985, page 377.

2-Cyanobenzene compounds Ia where A is a radical NR¹⁰—C(O)—R⁹ may be converted by known methods to compounds Ia where A is a radical NR¹⁰—C(S)—R⁹ by treatment with sulfurizing agents. Examples of suitable sulfurizing agents are organophosphorus sulfides such as Lawesson reagent, organotin sulfides or phosphorus(V) sulfides, see also J. March, Advanced Organic Synthesis, 3^{rd} edition, Wiley Interscience, 1985, p. 794 and literature cited therein. The reaction can be carried out in a solvent or in substance. The temperature required for the reaction is generally above room temperature and is in particular in the range from 50 to 200° C.

2-Cyanobenzene compounds Ia, in which A is a radical of the formula N═CR⁵R⁶ where R⁵ is a radical OR^{5a} where R^{5a} has the meanings mentioned above and R⁶ has the meanings mentioned above except for OR^{6a} and NR^{6b}R^{6c} may be obtained by reacting a cyanobenzenesulfonamide (II) (where R¹⁰ is H) with an orthoester (VII) in analogy to a procedure described in Chemische Berichte 1965, 623-628, see scheme 4. In scheme 4, the variables R¹, R², R³ and R⁴ have the meanings mentioned above and in particular the meanings mentioned as being preferred.

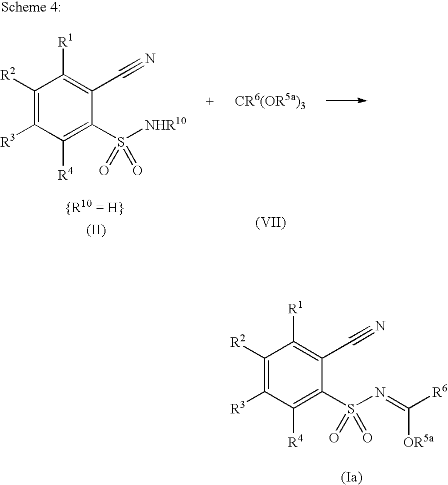

2-Cyanobenzene compounds Ia, in which A is a radical of the formula N═CR⁵R⁶ where R⁵ is a radical OR^{5a} and R⁶ is a radical OR^{6a}, where R^{5a} and R^{6a} are as defined above can be obtained by reacting a 2-cyanobenzene sulfonamide (II) (where R¹⁰ is H) with a carbonic acid orthoester (VIII) in a process similarly described in Journal of Organic Chemistry 1963, 28, 2902-2903, see scheme 5. In scheme 5 the variables R¹, R², R³R⁴, R^{5a} and R^{6a} have the meanings mentioned above and in particular the meanings mentioned as being preferred.

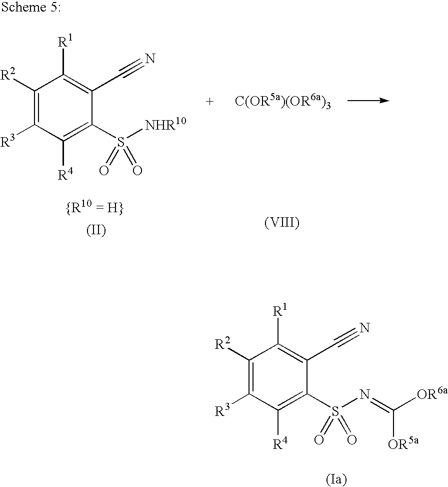

2-Cyanobenzene compounds Ia, wherein A is N═CR⁵R⁶ in which R⁵ is OR^{5a} and R⁶ is NR^{6b}R^{6c} where R^{5a}, R^{6b} and R^{6c} have the meanings mentioned above may be prepared by reacting the cyanobenzene compounds Ia (A is N═CR⁵R⁶ where R⁵ is OR^{5a} and R⁶ is OR^{6a} and which are obtainable according to scheme 5) with amines IX, in analogy to a procedure described in in J. Med. Chem. 1999, 42, 1235-1249. In scheme 6, the variables R¹, R², R³ and R⁴ have the meanings mentioned above and in particular the meanings mentioned as being preferred.

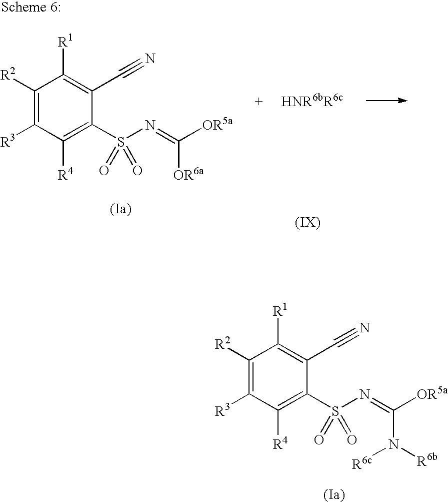

2-Cyanobenzene compounds Ia, wherein A is N═CR⁵R⁶ in which R⁵ is NR^{5b}R^{5c} where R^{5b} and R^{5c} are as defined above and R⁶ has the meanings mentioned above, except for OR^{6a} and NR^{6b}R^{6c}, may be obtained by reacting a cyanobenzene sulfonamide (II) (R¹⁰ is H) with amides (X) in analogy to J. Med. Chem. 2001, 44, 1085-1098, see scheme 7. It may be advantageous to use the amine (X) in an excess based on the amount of sulfonamide (II), e.g. the molar ratio of sulfonamide II to amine (X) is 1:1.01 to 1:3. The reaction is usually carried out in an inert organic solvent, such as alkanes, cycloalkanes or aromatic solvents, e.g. toluene. In scheme 7, the variables R¹, R², R³ and R⁴ have the meanings mentioned above and in particular the meanings mentioned as being preferred and R is C₁-C₆-alkyl, especially C₁-C₂-alkyl, in particular methyl.

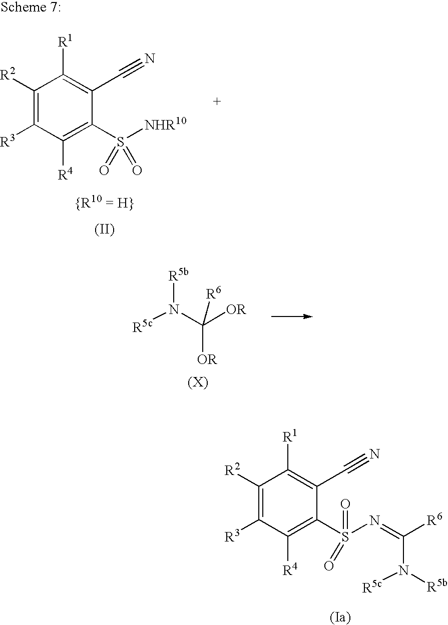

2-Cyanobenzene compounds Ia, in which A is a radical of the formula N═CR⁵R⁶ where R⁵ and R⁶ have the meanings mentioned above, except for R⁵═OR^{5a} or NR^{5b}R^{5c} and R⁶═OR^{6a} or NR^{6b}R^{6c}, may be prepared by reacting a sulfonamide (II) with ketones (XI) and a Lewis acid such as titanium(IV) chloride. The reaction is usually carried out in a polar solvent, for example C₁-C₄-alkanols such as methanol, ethanol, n-propanol or isopropanol, carboxamides such as N,N-dimethyl formamide, N,N-dimethyl acetamide or N-methylpyrrolidinone. The reaction of II with a ketone XI may be carried out analogously to the procedure described in Tetrahedron 1986, 42, 5649-5656.

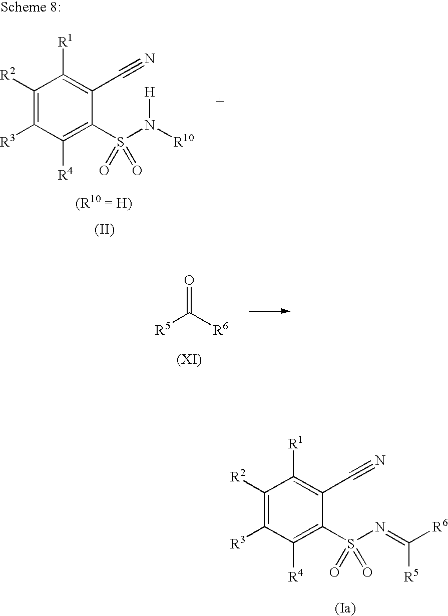

In scheme 8, the variables R¹, R², R³ and R⁴ have the meanings mentioned above and in particular the meanings mentioned as being preferred.

2-Cyanobenzene compounds Ia, wherein A is N═SR⁷R⁸ in which R⁷ and R⁸ have the meanings mentioned above, can be prepared reacting a sulfonamide (II) with sulfides (XII) and a chlorinating agent such as t-butyl-hypochlorite in the presence of a base, see scheme 9. Suitable bases are for example those bases mentioned above. The reaction is usually carried out in a polar solvent, for example C₁-C₄-alkanols such as methanol, ethanol, n-propanol or isopropanol, carboxamides such as N,N-dimethyl formamide, N,N-dimethyl acetamide or N-methylpyrrolidinone. The reaction of II with Xi can be carried out analogously to methods known from prior art, for example, in analogy to Tetrahedron 1986, 42, 5649-5656.

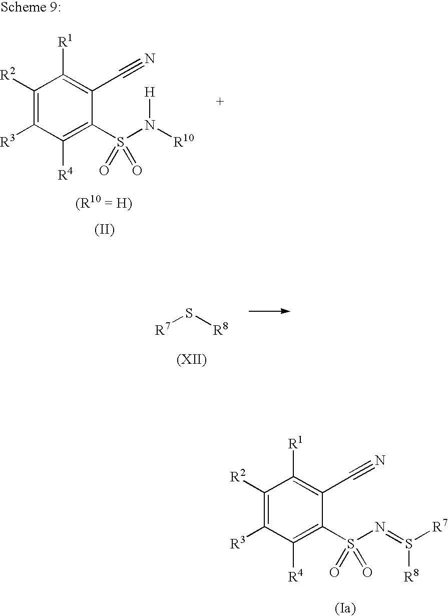

In scheme 9, the variables R¹, R², R³ and R⁴ have the meanings mentioned above and in particular the meanings mentioned as being preferred.

2-Cyanobenzene compounds Ia, wherein A is an N-bound 5-, 6- or 7-membered aromatic or non-aromatic unsaturated heterocycle as defined above, may be prepared by reacting a sulfonamide (II) with a 2,5-dialkoxytetrahydrofuran (XIII) which may be un-substituted or may carry 1, 2, 3 or 4 radicals R¹³ (where R¹³ has the meanings mentioned above), see scheme 10. The reaction is carried out in an acidic solvent such as carboxylic acids, for example C₁-C₄-carboxylic acids, e.g. acetic acid. The reaction of II with XIII can be carried out analogously to methods known from the literature, for example, as described in Synthetic Communications 1983, 13, 741-744.

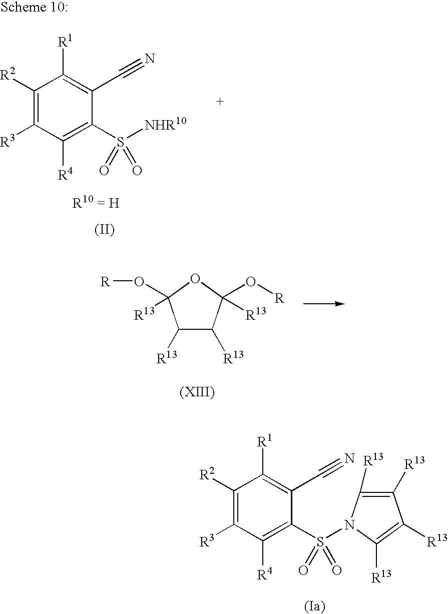

In scheme 10, the variables R¹, R², R³ and R⁴ have the meanings mentioned above and in particular the meanings mentioned as being preferred R is C₁-C₆-alkyl, especially C₁-C₂-alkyl, in particular methyl.

Alternatively, 2-cyanobenzene compounds Ia, wherein A is an N-bound 5-, 6- or 7-membered aromatic or non-aromatic unsaturated heterocycle as defined above, may be prepared by reacting a sulfonylchloride (IV) with an appropriate 5-, 6- or 7-membered aromatic or non-aromatic unsaturated N containing heterocycle (XIV) in the presence of an inorganic base, see scheme 11. Suitable bases are, for example, alkali metal or alkaline earth metal hydroxides, bicarbonates or carbonates such as lithium hydroxide, lithium bicarbonate or lithium carbonate, sodium hydroxide, sodium bicarbonate or sodium carbonate, potassium hydroxide, potassium bicarbonate or potassium carbonate, calcium hydroxide, calcium bicarbonate or calcium carbonate, or magnesium hydroxide, magnesium bicarbonate or magnesium carbonate. The reaction is carried out under phase transfer catalysis conditions. Suitable for use as phase-transfer catalysts are quaternary ammonium or phosphonium salts. Suitable compounds which may be mentioned are the following: tetraalkyl-(C₁-C₁₈)-ammonium chlorides, bromides or fluorides, N-benzyltrialkyl-(C₁-C₁₈)-ammonium chlorides, bromides or fluorides, tetraalkyl-(C₁-C₁₈)-phosphonium chlorides or bromides, tetraphenylphosphonium chloride or bromide, (phenyl)ₒ(C₁-C₁₈-alkyl)ₚ-phosphonium chlorides or bromides, where o=1 to 3, p=3 to 1 and o+p=4. The reaction of IV with XIV can be carried out analogously to methods known from the literature, for example, as described in Can. J. Chem 1985, 63, 896-902.

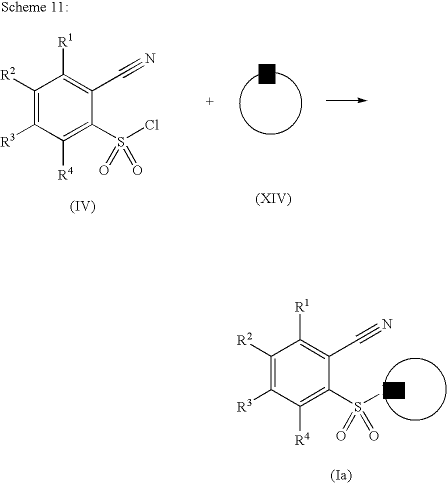

In scheme 11, the variables R¹, R², R³ and R⁴ have the meanings mentioned above and in particular the meanings mentioned as being preferred.

In a similar way, 2-cyanobenzene compounds Ia, wherein A is an optionally substituted imidazole may be prepared in analogy to a method described in Indian Journal of Heterocyclic Chemistry 2003, 13, 79-80; 2-cyanobenzene compounds Ia, wherein A is an optionally substituted pyrazole, may be prepared in analogy to a method described in Heterocycles 1988, 27, 2443-2457; or 2-cyanobenzene compounds Ia, wherein A is an optionally substituted triazole may be prepared in analogy to a method described in Bioorganic & Medicinal Chemistry 2004, 2317-2333.

The starting material of the formula II as well as the starting material of the formula IV required for preparing the compounds Ia are known from the literature, for example from WO 2005/035486 cited at the introductory part or they can be prepared in accordance with the literature cited therein.

2-Cyanobenzene compounds Ib, i.e. compounds I in which m=0, may be prepared by the processes as described in schemes 1 to 11, but using the corresponding sulfenylamide compound XV and sulfenylhalogenide compound XVI, respectively.

The sulfenylamide compounds of the formula XV, may be prepared, for example, by reacting a 2-cyanobenzenesulfenylhalide, (XVI), especially a 2-cyanobenzenesulfenylchloride, with ammonia or a primary amine (XVII) by analogy to a process described in Journal of Organic Chemistry 1977, V0. 42, No. 4, pp. 597-600 or in Journal of Medicinal Chemistry 2001, Vol. 44, No. 13, pp. 2253-2258, see Scheme 12.

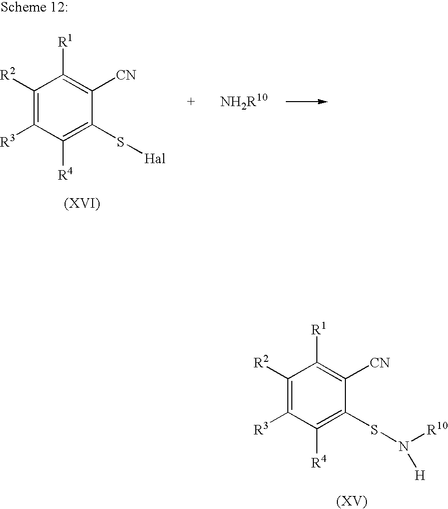

In scheme 12, the variables R¹, R², R³, R⁴ and R¹⁰ have the meanings mentioned above and in particular the meanings mentioned as being preferred and Hal is halogen, in particular chlorine. The reaction of a sulfenylhalide XVI with an amine XVII is usually carried out at a reaction temperature ranging from 0° C. to the boiling point of the solvent, preferably from 0 to 30° C.

In general, the amine XVII is employed in an at least equimolar amount, preferably at least 2-fold molar excess, based on the sulfenylhalide XVI, to bind the hydrogen halide formed. It may be advantageous to employ the primary amine XVII in an up to 6-fold molar excess, based on the sulfenylhalide XVI.

The reaction is usually carried out in the presence of a solvent. Suitable solvents are polar solvents which are inert under the reaction conditions, for example C₁-C₄-alkanols such as methanol, ethanol, n-propanol or isopropanol, dialkyl ethers such as diethyl ether, diisopropyl ether or methyl tert-butyl ether, cyclic ethers such as dioxane or tetrahydrofuran, acetonitrile, carboxamides such as N,N-dimethyl formamide, N,N-dimethyl acetamide or N-methylpyrrolidinone, water, (provided the sulfenylhalide XVI is sufficiently resistent to hydrolysis under the reaction conditions used) or a mixture thereof.

The sulfenylhalide compounds XVI may be prepared, for example by the process as described hereinafter comprising steps a) and b), see scheme 13.

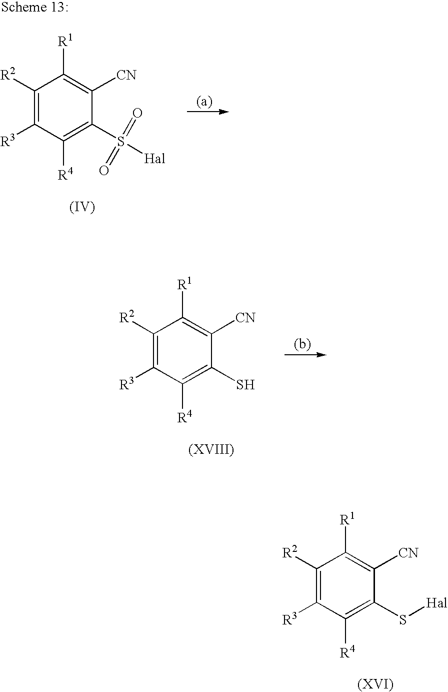

In scheme 13, the variables R¹, R², R³ and R⁴ have the meanings mentioned above and in particular the meanings mentioned as being preferred and Hal is halogen, in particular chlorine.
a) Reduction of a sulfonylhalide IV to the corresponding thiol XVIIII using a suitable reducing agent. Suitable reducing agents include (1) tris(2-carboxyethyl)phosphine in a mixture of dioxane and water as described in Synthetic Communications 2003, Vol. 33, No. 20, pp. 3503-3511; or (2) triphenylphosphine with or without the addition of iodine in an aromatic solvent like benzene or toluene as described in Bulletin of the Chemical Society of Japan 1983, Vol. 56, No. 12, pp. 3802-3812; or (3) zinc in combination with dichlorodimethylsilane, dimethylacetamide in a chlorinated hydrocarbon like dichloroethane as described in Tetrahedron Letters 1999, Vol. 40, pp. 3179-3182.
b) Direct conversion of the thiol XVIII to the sulfenylhalide XVI by oxidative halogenation, for example oxidative chlorination using N-chlorosuccinimide in a chlorinated hydrocarbon such as dichloromethane as described in the Journal of Organic Chemistry 1985, Vol. 50, No. 19 pp. 3592-3595.

2-Cyanobenzene compounds Ic, i.e. compounds I in which m=1, may be prepared by following the procedures analogous to those described in schemes 1 to 11, but using the sulfinylamide compound XIX and sulfinylhalide compound XX, respectively.

The sulfinylamide compounds of the formula XIX can be prepared, for example, by reacting a 2-cyanobenzenesulfinylhalide (XX) with ammonia or a primary amine (XVII) in analogy to a process described in Journal of Organic Chemistry 1983, Vol. 48 pp. 4803-4807, see scheme 14.

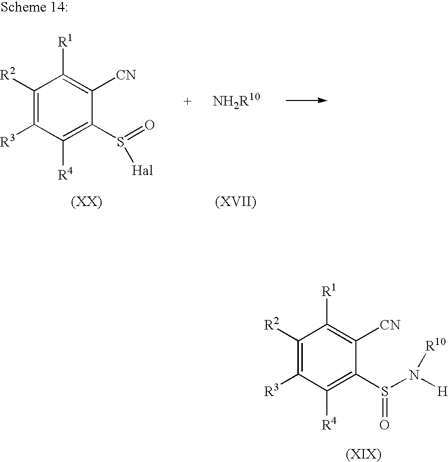

In scheme 14, the variables R¹, R², R³ and R⁴ have the meanings mentioned above and in particular the meanings mentioned as being preferred and Hal is halogen, in particular chlorine. In general, the amine XVII is employed in an at least equimolar amount, preferably at least 2-fold molar excess, based on the sulfinylhalide XX, to bind the hydrogen halide formed. It may be advantageous to employ the primary amine XVII in an up to 6-fold molar excess, based on the sulfinylhalide XX. The reaction of XVII with XX is usually carried out at a reaction temperature ranging from 0° C. to the boiling point of the solvent, preferably from 0 to 30° C.

The reaction of a sulfinylhalide XX with an amine XVII is usually carried out in the presence of a solvent. Suitable solvents are polar solvents which are inert under the reaction conditions, for example C₁-C₄-alkanols such as methanol, ethanol, n-propanol or isopropanol, dialkyl ethers such as diethyl ether, diisopropyl ether or methyl tert-butyl ether, cyclic ethers such as dioxane or tetrahydrofuran, acetonitrile, carboxamides such as N,N-dimethyl formamide, N,N-dimethyl acetamide or N-methylpyrrolidinone, water, (provided the sulfinylhalide XX is sufficiently resistent to hydrolysis under the reaction conditions used) or a mixture thereof.

The sulfinylhalide compounds XX may be prepared for example by the process as described hereinafter, (see scheme 15) comprising steps (a) and (b):

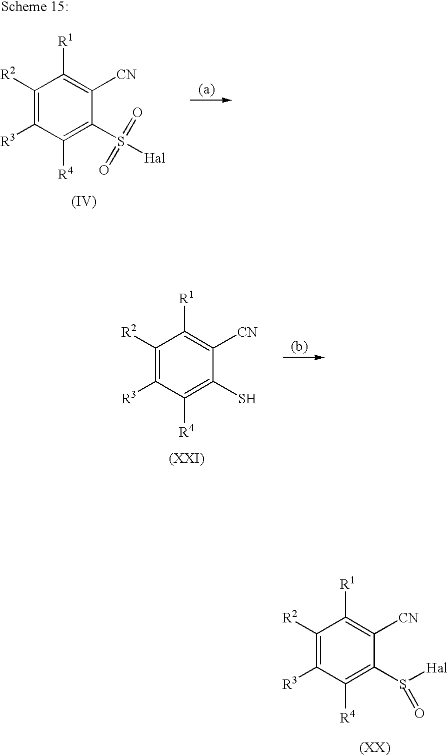

In scheme 15, the variables R¹, R², R³ and R⁴ have the meanings mentioned above and in particular the meanings mentioned as being preferred and Hal is halogen, in particular chlorine.
(a) Reduction of a sulfonylhalide IV to the corresponding thiol XXI using a suitable reducing agent as described in step a) in scheme 13.
(b) Conversion of the thiol XXI into the sulfinylhalide XX by oxidative halogenation, for example oxidative chlorination using sulfuryl chloride in acetic acid as described in Synthesis 1987, No. 1, pp. 72-73.

If individual compounds cannot be prepared via the above-described routes, they can be prepared by derivatization of other compounds I or by customary modifications of the synthesis routes described.

The reaction mixtures are worked up in the customary manner, for example by mixing with water, separating the phases, and, if appropriate, purifying the crude products by chromatography, for example on alumina or on silica gel. Some of the intermediates and end products may be obtained in the form of colorless or pale brown viscous oils which are freed or purified from volatile components under reduced pressure and at moderately elevated temperature. If the intermediates and end products are obtained as solids, they may be purified by recrystallization or digestion.

The compounds of the formula I and their salts are in particular suitable for efficiently controlling arthropodal pests such as arachnids, myriapedes and insects as well as nematodes.

In particular, they are suitable for controlling insect pests, such as insects from the order of

Lepidoptera: for example Agrotis ypsilon, Agrotis segetum, Alabama argillacea, Anticarsia gemmatalis, Argyresthia conjugella, Autographa gamma, Bupalus piniarius, Cacoecia murinana, Capua reticulana, Chematobia brumata, Choristoneura fumiferana, Choristoneura occidentalis, Cirphis unipuncta, Cydia pomonella, Dendrolimus pini, Diaphania nitidalis, Diatraea grandiosella, Earias insulana, Elasmopalpus lignosellus, Eupoecilia ambiguella, Evetria bouliana, Feltia subterranea, Galleria mellonella, Grapholitha funebrana, Grapholitha molesta, Heliothis armigera, Heliothis virescens, Heliothis zea, Hellula undalis, Hibernia defoliaria, Hyphantria cunea, Hyponomeuta malinellus, Keiferia lycopersicella, Lambdina fiscellaria, Laphygma exigua, Leucoptera coffeella, Leucoptera scitella, Lithocolletis blancardella, Lobesia botrana, Loxostege sticticalis, Lymantria dispar, Lymantria monacha, Lyonetia clerkella, Malacosoma neustria, Mamestra brassicae, Orgyia pseudotsugata, Ostrinia nubilalis, Panolis flammea, Pectinophora gossypiella, Peridroma saucia, Phalera bucephala, Phthorimaea operculella, Phyllocnistis citrella, Pieris brassicae, Plathypena scabra, Plutella xylostella, Pseudoplusia includens, Rhyacionia frustrana, Scrobipalpula absoluta, Sitotroga cerealella, Sparganothis pilleriana, Spodoptera eridania, Spodoptera frugiperda, Spodoptera littoralis, Spodoptera litura, Thaumatopoea pityocampa, Tortrix viridana, Trichoplusia ni and Zeiraphera canadensis,

Coleoptera (beetles), for example Agrilus sinuatus, Agriotes lineatus, Agriotes obscurus, Amphimallus solstitialis, Anisandrus dispar, Anthonomus grandis, Anthonomus pomorum, Atomaria linearis, Blastophagus piniperda, Blitophaga undata, Bruchus rufimanus, Bruchus pisorum, Bruchus lentis, Byctiscus betulae, Cassida nebulosa, Cerotoma trifurcata, Ceuthorrhynchus assimilis, Ceuthorrhynchus napi, Chaetocnema tibialis, Conoderus vespertinus, Crioceris asparagi, Diabrotica longicornis, Diabrotica 12-punctata, Diabrotica virgifera, Epilachna varivestis, Epitrix hirtipennis, Eutinobothrus brasiliensis, Hylobius abietis, Hypera brunneipennis, Hypera postica, Ips typographus, Lema bilineata, Lema melanopus, Leptinotarsa decemlineata, Limonius californicus, Lissorhoptrus oryzophilus, Melanotus communis, Meligethes aeneus, Melolontha hippocastani, Melolontha melolontha, Oulema oryzae, Ortiorrhynchus sulcatus, Otiorrhynchus ovatus, Phaedon cochleariae, Phyllotreta chrysocephala, Phyllophaga sp., Phyllopertha horticola, Phyllotreta nemorum, Phyllotreta striolata, Popillia japonica, Sitona lineatus and Sitophilus granaria,

Diptera, for example Aedes aegypti, Aedes vexans, Anastrepha ludens, Anopheles maculipennis, Ceratitis capitata, Chrysomya bezziana, Chrysomya hominivorax, Chrysomya macellaria, Contarinia sorghicola, Cordylobia anthropophaga, Culex pipiens, Dacus cucurbitae, Dacus oleae, Dasineura brassicae, Fannia canicularis, Gasterophilus intestinalis, Glossina morsitans, Haematobia irritans, Haplodiplosis equestris, Hylemyia platura, Hypoderma lineata, Liriomyza sativae, Liriomyza trifolii, Lucilia caprina, Lucilia cuprina, Lucilia sericata, Lycoria pectoralis, Mayetiola destructor, Musca domestica, Muscina stabulans, Oestrus ovis, Oscinella frit, Pegomya hysocyami, Phorbia antiqua, Phorbia brassicae, Phorbia coarctata, Rhagoletis cerasi, Rhagoletis pomonella, Tabanus bovinus, Tipula oleracea and Tipula paludosa,

Thysanoptera (thrips), e.g. Dichromothrips spp., Frankliniella fusca, Frankliniella occidentalis, Frankliniella tritici, Scirtothrips citri, Thrips oryzae, Thrips palmi and Thrips tabaci,

Hymenoptera e.g. Athalia rosae, Atta cephalotes, Atta sexdens, Atta texana, Hoplocampa minuta, Hoplocampa testudinea, Monomorium pharaonis, Solenopsis geminata and Solenopsis invicta,

Heteroptera, e.g. Acrosternum hilare, Blissus leucopterus, Cyrtopeltis notatus, Dysdercus cingulatus, Dysdercus intermedius, Eurygaster integriceps, Euschistus impictiventris, Leptoglossus phyllopus, Lygus lineolaris, Lygus pratensis, Nezara viridula, Piesma quadrata, Solubea insularis and Thyanta perditor,

Homoptera (in particular aphids), e.g. Acyrthosiphon onobrychis, Adelges laricis, Aphidula nasturtii, Aphis craccivora, Aphis fabae, Aphis forbesi, Aphis pomi, Aphis gossypii, Aphis grossulariae, Aphis schneideri, Aphis spiraecola, Aphis sambuci, Acyrthosiphon pisum, Aulacorthum solani, Bemisa tabaci, Bemisa argentifolii, Brachycaudus cardui, Brachycaudus helichrysi, Brachycaudus persicae, Brachycaudus prunicola, Brevicoryne brassicae, Capitophorus horni, Cerosipha gossypii, Chaetosiphon fragaefolii, Cryptomyzus ribis, Dreyfusia nordmannianae, Dreyfusia piceae, Dysaphis radicola, Dysaulacorthum pseudosolani, Dysaphis plantaginea, Dysaphis pyri, Empoasca fabae, Hyalopterus pruni, Hyperomyzus/actucae, Macrosiphum avenae, Macrosiphum euphorbiae, Macrosiphon rosae, Megoura viciae, Melanaphis pyrarius, Metopolophium dirhodum, Myzodes persicae, Myzus ascalonicus, Myzus cerasi, Myzus varians, Nasonovia ribis-nigri, Nilaparvata lugens, Pemphigus bursarius, Perkinsiella saccharicida, Phorodon humuli, Psylla mali, Psylla piri, Rhopalomyzus ascalonicus, Rhopalosiphum maidis, Rhopalosiphum padi, Rhopalosiphum insertum, Sappaphis mala, Sappaphis mali, Schizaphis graminum, Schizoneura lanuginosa, Sitobion avenae, Trialeurodes vaporariorum, Toxoptera aurantiiand, and Viteus vitifolii,

Isoptera (termites), e.g. Calotermes flavicollis, Leucotermes flavipes, Reticulitermes lucifugus und Termes natalensis,

Orthoptera, e.g. Acheta domestica, Blatta orientalis, Blattella germanica, Forficula auricularia, Gryllotalpa gryllotalpa, Locusta migratoria, Melanoplus bivittatus, Melanoplus femur-rubrum, Melanoplus mexicanus, Melanoplus sanguinipes, Melanoplus spretus, Nomadacris septemfasciata, Periplaneta americana, Schistocerca americana, Schistocerca peregrina, Stauronotus maroccanus and Tachycines asynamorus, and

Collembola (springtails), e.g. Onychiurus ssp.

They are also suitable for controlling Nematodes: plant parasitic nematodes such as root knot nematodes, Meloidogyne hapla, Meloidogyne incognita, Meloidogyne javanica, and other Meloidogyne species; cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Belonolaimus longicaudatus and other Belonolaimus species; Pine nematodes, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species.

The compounds of the formula I and their salts are also useful for controlling arachnids (Arachnoidea), such as acarians (Acarina), e.g. of the families Argasidae, Ixodidae and Sarcoptidae, such as Amblyomma americanum, Amblyomma variegatum, Argas persicus, Boophilus annulatus, Boophilus decoloratus, Boophilus microplus, Dermacentor silvarum, Hyalomma truncatum, Ixodes ricinus, Ixodes rubicundus, Ornithodorus moubata, Otobius megnini, Dermanyssus gallinae, Psoroptes ovis, Rhipicephalus appendiculatus, Rhipicephalus evertsi, Sarcoptes scabiei, and Eriophyidae spp. such as Aculus schlechtendali, Phyllocoptrata oleivora and Eriophyes sheldoni; Tarsonemidae spp. such as Phytonemus pallidus and Polyphagotarsonemus latus; Tenuipalpidae spp. such as Brevipalpus phoenicis; Tetranychidae spp. such as Tetranychus cinnabarinus, Tetranychus kanzawai, Tetranychus pacificus, Tetranychus telarius and Tetranychus urticae, Panonychus ulmi, Panonychus citri, and oligonychus pratensis.

Compounds of the formula I are particularly useful for controlling insects, preferably sucking or piercing insects such as insects from the genera Thysanoptera, Hymenoptera, Orthoptera and Homptera, in particular the following species:

Thysanoptera (thrips): Frankliniella fusca, Frankliniella occidentalis, Frankliniella tritici, Scirtothrips citri, Thrips oryzae, Thrips palmi and Thrips tabaci,

Hymenoptera: Athalia rosae, Atta cephalotes, Atta sexdens, Atta texana, Hoplocampa minuta, Hoplocampa testudinea, Monomorium pharaonis, Solenopsis geminata and Solenopsis invicta,

Orthoptera: Acheta domestica, Blatta orientalis, Blattella germanica, Forficula auricularia, Gryllotalpa gryllotalpa, Locusta migratoria, Melanoplus bivittatus, Melanoplus femur-rubrum, Melanoplus mexicanus, Melanoplus sanguinipes, Melanoplus spretus, Nomadacris septemfasciata, Periplaneta americana, Schistocerca americana, Schistocerca peregrina, Stauronotus maroccanus and Tachycines asynamorus;

Homoptera, in particular aphids: Acyrthosiphon onobrychis, Adelges laricis, Aphidula nasturtii, Aphis fabae, Aphis forbesi, Aphis pomi, Aphis gossypii, Aphis grossulariae, Aphis schneideri, Aphis spiraecola, Aphis sambuci, Acyrthosiphon pisum, Aulacorthum solani, Brachycaudus cardui, Brachycaudus helichrysi, Brachycaudus persicae, Brachycaudus prunicola, Brevicoryne brassicae, Capitophorus horni, Cerosipha gossypii, Chaetosiphon fragaefolii, Cryptomyzus ribis, Dreyfusia nordmannianae, Dreyfusia piceae, Dysaphis radicola, Dysaulacorthum pseudosolani, Dysaphis plantaginea, Dysaphis pyri, Empoasca fabae, Hyalopterus pruni, Hyperomyzus lactucae, Macrosiphum avenae, Macrosiphum euphorbiae, Macrosiphon rosae, Megoura viciae, Melanaphis pyrarius, Metopolophium dirhodum, Myzodes persicae, Myzus ascalonicus, Myzus cerasi, Myzus varians, Nasonovia ribis-nigri, Nilaparvata lugens, Pemphigus bursarius, Perkinsiella saccharicida, Phorodon humuli, Psylla mali, Psylla piri, Rhopalomyzus ascalonicus, Rhopalosiphum maidis, Rhopalosiphum padi, Rhopalosiphum insertum, Sappaphis mala, Sappaphis mali, Schizaphis graminum, Schizoneura lanuginosa, Sitobion avenae, Trialeurodes vaporariorum, Toxoptera aurantiiand, and Viteus vitifolii;

Compounds of the formula I are particularly useful for controlling insects of the orders Homoptera and Thysanoptera and more preferably for controlling aphids.

For use in a method according to the present invention, the compounds I can be converted into the customary formulations, e.g. solutions, emulsions, suspensions, dusts, powders, pastes, granules and directly sprayable solutions. The use form depends on the particular purpose and application method. Formulations and application methods are chosen to ensure in each case a fine and uniform distribution of the compound of the formula I according to the present invention.

The formulations are prepared in a known manner, e.g. by extending the active ingredient with solvents and/or carriers, if desired using surfactants, i.e. emulsifiers and dispersants and other formulation auxiliaries.

Solvents/carriers, which are suitable, are e.g.:
solvents such as water, aromatic solvents (for example Solvesso products, xylene and the like), paraffins (for example mineral fractions), alcohols (for example methanol, butanol, pentanol, benzyl alcohol), ketones (for example cyclohexanone, gamma-butyrolactone), pyrrolidones (NMP, NOP), acetates (glycol diacetate), alkyl lactates, lactones such as g-butyrolactone, glycols, fatty acid dimethylamides, fatty acids and fatty acid esters, triglycerides, oils of vegetable or animal origin and modified oils such as alkylated plant oils. In principle, solvent mixtures may also be used.
carriers such as ground natural minerals and ground synthetic minerals, such as silica gels, finely divided silicic acid, silicates, talc, kaolin, attaclay, limestone, lime, chalk, bole, loess, clay, dolomite, diatomaceous earth, calcium sulfate and magnesium sulfate, magnesium oxide, ground synthetic materials, fertilizers, such as, for example, ammonium sulfate, ammonium phosphate, ammonium nitrate, ureas and products of vegetable origin, such as cereal meal, tree bark meal, wood meal and nutshell meal, cellulose powders and other solid carriers.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignin-sulfite waste liquors and methylcellulose.

Suitable auxiliaries comprise stabilizers, buffers, antioxidants, biocides, antifoams, thickeners, antifreeze and the like.

Suitable thickeners are compounds which confer a pseudoplastic flow behavior to the formulation, i.e. high viscosity at rest and low viscosity in the agitated stage. Mention may be made, in this connection, for example, of cammercial thickeners based on polysaccharides, such as Xanthan Gum® (Kelzan® from Kelco), Rhodopol® 23 (Rhone Poulenc) or Veegum® (from R. T. Vanderbilt), or organic phyllosilicates, such as Attaclay® (from Engelhardt). Antifoam agents suitable for the dispersions according to the invention are, for example, silicone emulsions (such as, for example, Silikon® SRE, Wacker or Rhodorsil® from Rhodia), long-chain alcohols, fatty acids, organofluorine compounds and mixtures thereof. Biocides can be added to stabilize the compositions according to the invention against attack by microorganisms. Suitable biocides are, for example, based on isothiazolones such as the compounds marketed under the trademarks Proxel® from Avecia (or Arch) or Acticide® RS from Thor Chemie and Kathon® MK from Rohm & Haas. Suitable antifreeze agents are organic polyols, for example ethylene glycol, propylene glycol or glycerol. These are usually employed in amounts of not more than 10% by weight, based on the total weight of the active compound composition. If appropriate, the active compound compositions according to the invention may comprise 1 to 5% by weight of buffer, based on the total amount of the formulation prepared, to regulate the pH, the amount and type of the buffer used depending on the chemical properties of the active compound or the active compounds. Examples of buffers are alkali metal salts of weak inorganic or organic acids, such as, for example, phosphoric acid, boronic acid, acetic acid, propionic acid, citric acid, fumaric acid, tartaric acid, oxalic acid and succinic acid.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dusts can be prepared by mixing or concomitantly grinding the active substances with a solid carrier.

Granules, for example coated granules, impregnated granules and homogeneous granules, can be prepared by binding the active ingredients to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active ingredient. The active ingredients are employed in a purity of from 90% to 100%, preferably 95% to 100% (according to NMR spectrum).

The following are examples of formulations:

1. Products for Dilution with Water

### A Soluble Concentrates (SL)

10 parts by weight of a compound according to the invention are dissolved in water or in a water-soluble solvent. As an alternative, wetters or other auxiliaries are added. The active ingredient dissolves upon dilution with water.

### B Dispersible Concentrates (DC)

20 parts by weight of a compound according to the invention are dissolved in cyclohexanone with addition of a dispersant, for example polyvinylpyrrolidone. Dilution with water gives a dispersion.

### C Emulsifiable Concentrates (EC)

15 parts by weight of a compound according to the invention are dissolved in xylene with addition of calcium dodecylbenzenesulfonate and castor oil ethoxylate (in each case 5% strength). Dilution with water gives an emulsion.

### D Emulsions (EW, EO)

40 parts by weight of a compound according to the invention are dissolved in xylene with addition of calcium dodecylbenzenesulfonate and castor oil ethoxylate (in each case 5% strength). This mixture is introduced into water by means of an emulsifier (Ultraturrax) and made into a homogeneous emulsion. Dilution with water gives an emulsion.

### E Suspensions (SC, OD)

In an agitated ball mill, 20 parts by weight of a compound according to the invention are milled with addition of dispersant, wetters and water or an organic solvent to give a fine active ingredient suspension. Dilution with water gives a stable suspension of the active ingredient.

### F Water-Dispersible Granules and Water-Soluble Granules (WG, SG)

50 parts by weight of a compound according to the invention are ground finely with addition of dispersants and wetters and made into water-dispersible or water-soluble granules by means of technical appliances (for example extrusion, spray tower, fluidized bed). Dilution with water gives a stable dispersion or solution of the active ingredient.

### G Water-Dispersible Powders and Water-Soluble Powders (WP, SP)

75 parts by weight of a compound according to the invention are ground in a rotor-stator mill with addition of dispersant, wetters and silica gel. Dilution with water gives a stable dispersion or solution with the active ingredient.

### 2. Products to be Applied Undiluted

### H Dustable Powders (DP)

5 parts by weight of a compound according to the invention are ground finely and mixed intimately with 95% of finely divided kaolin. This gives a dustable product.

### I Granules (GR, FG, GG, MG)

0.5 parts by weight of a compound according to the invention is ground finely and associated with 95.5% carriers. Current methods are extrusion, spray drying or the fluidized bed. This gives granules to be applied undiluted.

### J ULV Solutions (UL)

10 parts by weight of a compound according to the invention are dissolved in an organic solvent, for example xylene. This gives a product to be applied undiluted.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active ingredient concentrations in the ready-to-use products can be varied within relatively wide ranges. In general, they are from 0.0001 to 10%, preferably from 0.01 to 1%.

The active ingredients may also be used successfully in the ultra-low-volume process (ULV), it being possible to apply formulations comprising over 95% by weight of active ingredient, or even to apply the active ingredient without additives.

In the method of this invention compounds I may be applied with other active ingredients, for example with other pesticides, insecticides, herbicides, fertilizers such as ammonium nitrate, urea, potash, and superphosphate, phytotoxicants and plant growth regulators, safeners and nematicides. These additional ingredients may be used sequentially or in combination with the above-described compositions, if appropriate also added only immediately prior to use (tank mix). For example, the plant(s) may be sprayed with a composition of this invention either before or after being treated with other active ingredients.

The following list of pesticides together with which the compounds according to the invention can be used and with which potential synergistic effects might be produced is intended to illustrate the possible combinations, but not to impose any limitation:

(1) Organo(thio)phosphates: acephate, azamethiphos, azinphos-methyl, chlorpyrifos, chlorpyrifos-methyl, chlorfenvinphos, diazinon, dichlorvos, dicrotophos, dimethoate, disulfoton, ethion, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, sulprophos, tetrachlorvinphos, terbufos, triazophos, trichlorfon;
(2) Carbamates: alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, triazamate;
(3) Pyrethroids: allethrin, bifenthrin, cyfluthrin, cyhalothrin, cyphenothrin, cypermethrin, alpha-cypermethrin, beta-cypermeth rin, zeta-cypermethri n, deltamethrin, empenthrin, esfenvalerate, etofenprox, fenpropathrin, fenvalerate, imiprothrin, lambda-cyhalothrin, permethrin, prallethrin, pyrethrin I and II, resmethrin, silafluofen, tau-fluvalinate, tefluthrin, tetramethrin, tralomethrin, transfluthrin, profluthrin, dimefluthrin;
(4) Growth regulators: a) chitin synthesis inhibitors: benzoylureas: chlorfluazuron, diflubenzuron, flucycloxuron, flufenoxuron, hexaflumuron, lufenuron, novaluron, teflubenzuron, triflumuron; buprofezin, diofenolan, hexythiazox, etoxazole, clofentazine; b) ecdysone antagonists: halofenozide, methoxyfenozide, tebufenozide, azadirachtin; c) juvenoids: pyriproxyfen, methoprene, fenoxycarb; d) lipid biosynthesis inhibitors: spirodiclofen, spiromesifen, spirotetramat;
(5) Nicotinic receptor agonists/antagonists compounds: clothianidin, dinotefuran, imidacloprid, thiamethoxam, nitenpyram, acetamiprid, thiacloprid and AKD-1022,
(6) GABA antagonist compounds: acetoprole, endosulfan, ethiprole, fipronil, vaniliprole, pyrafluprole, pyriprole, the phenylpyrazole compound of formula Γ²

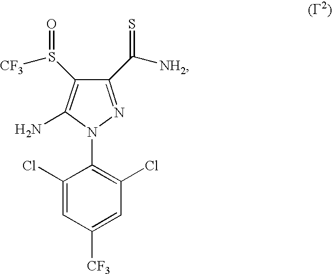

(7) Macrocyclic lactone insecticides: abamectin, emamectin, milbemectin, lepimectin, spinosad;
(8) METI I compounds: fenazaquin, pyridaben, tebufenpyrad, tolfenpyrad, flufenerim;
(9) METI II and III compounds: acequinocyl, fluacyprim, hydramethylnon;
(10) Uncoupler compounds: chlorfenapyr;
(11) Oxidative phosphorylation inhibitor compounds: cyhexatin, diafenthiuron, fenbutatin oxide, propargite;
(12) Moulting disruptor compounds: cyromazine;
(13) Mixed Function Oxidase inhibitor compounds: piperonyl butoxide;
(14) Sodium channel blocker compounds: indoxacarb, metaflumizone,
(15) Various: amitraz, benclothiaz, bifenazate, cartap, flonicamid, pyridalyl, pymetrozine, sulfur, thiocyclam, flubendiamide, cyenopyrafen, flupyrazofos, cyflumetofen, amidoflumet, pyrifluquinazon, N—R′-2,2-dihalo-1-R″cyclo-propanecarboxamide-2-(2,6-dichloro-α,α,α-tri-fluoro-p-tolyl)hydrazone or N—R′-2,2-di(R′″)propionamide-2-(2,6-dichloro-α,α,α-trifluoro-p-tolyl)-hydrazone, wherein R′ is methyl or ethyl, halo is chloro or bromo, R″ is hydrogen or methyl and R′″ is methyl or ethyl, anthranilamide compounds such as chlorantraniliprole or the compound of formula Γ⁵

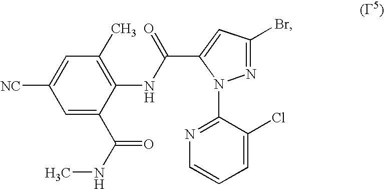

and malononitrile compounds as described in JP 2002 284608, WO 02/89579, WO 02/90320, WO 02/90321, WO 04/06677, WO 04/20399, JP 2004 99597, WO 05/68423, WO 05/68432, WO 05/63694 or WO 05/63694, especially the malononitrile compounds CF₃(CH₂)₂C(CN)₂CH₂(CF₂)₃CF₂H, CF₃(CH₂)₂C(CN)₂CH₂(CF₂)₅CF₂H, CF₃(CH₂)₂C(CN)₂(CH₂)₂C(CF₃)₂F, CF₃(CH₂)₂C(CN)₂(CH₂)₂(CF₂)₃CF₃, CF₂H(CF₂)₃CH₂C(CN)₂CH₂(CF₂)₃CF₂H, CF₃(CH₂)₂C(CN)₂CH₂(CF₂)₃CF₃, CF₃(CF₂)₂CH₂C(CN)₂CH₂(CF₂)₃CF₂H, and CF₃CF₂CH₂C(CN)₂CH₂(CF₂)₃CF₂H.

The commercially available compounds of the group (1) to (15) may be found in The Pesticide Manual, 13^{th} Edition, British Crop Protection Council (2003) among other publications.

Thioamides of formula Γ² and their preparation have been described in WO 98/28279. Lepimectin is known from Agro Project, PJB Publications Ltd, November 2004. Benclothiaz and its preparation have been described in EP-A1454621. Methidathion and Paraoxon and their preparation have been described in Farm Chemicals Handbook, Volume 88, Meister Publishing Company, 2001. Acetoprole and its preparation have been described in WO 98/28277. Metaflumizone and its preparation have been described in EP-A1 462 456. Flupyrazofos has been described in Pesticide Science 54, 1988, p. 237-243 and in U.S. Pat. No. 4,822,779. Pyrafluprole and its preparation have been described in JP 2002193709 and in WO 01/00614. Pyriprole and its preparation have been described in WO 98/45274 and in U.S. Pat. No. 6,335,357. Amidoflumet and its preparation have been described in U.S. Pat. No. 6,221,890 and in JP 21010907. Flufenerim and its preparation have been described in WO 03/007717 and in WO 03/007718. Cyflumetofen and its preparation have been described in WO 04/080180. The aminoquinazolinone compound pyrifluquinazon has been described in EP A 109 7932.

Anthranilamides such as chlorantraniliprole and the compound of formula Γ⁵ and their preparation have been described in WO 01/70671; WO 02/48137; WO 03/24222, WO 03/15518, WO 04/67528; WO 04/33468; and WO 05/118552. The malononitrile compounds CF₃(CH₂)₂C(CN)₂CH₂(CF₂)₃CF₂H, CF₃(CH₂)₂C(CN)₂CH₂(CF₂)₅CF₂H, CF₃(CH₂)₂C(CN)₂(CH₂)₂C(CF₃)₂F, CF₃(CH₂)₂C(CN)₂(CH₂)₂(CF₂)₃CF₃, CF₂H(CF₂)₃CH₂C(CN)₂CH₂(CF₂)₃CF₂H, CF₃(CH₂)₂C(CN)₂CH₂(CF₂)₃CF₃, CF₃(CF₂)₂C H₂C(CN)₂CH₂(CF₂)₃CF₂H, and CF₃CF₂CH₂C(CN)₂CH₂(CF₂)₃CF₂H have been described in WO 05/63694.

In the methods according to the invention the pests are controlled by contacting the target parasite/pest, its food supply, habitat, breeding ground or its locus with a pesticidally effective amount of compounds of formula I or with a salt thereof or with a composition containing a pesticidally effective amount of a compound of formula I or a salt thereof.

“Locus” means a habitat, breeding ground, plant, seed, soil, area, material or environment in which a pest or parasite is growing or may grow.

In general, “pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions such as desired pesticidal effect and duration, weather, target species, locus, mode of application, and the like.

The compounds of the invention can also be applied preventively to places at which occurrence of the pests is expected.

The compounds of formula I may be also used to protect growing plants from attack or infestation by pests by contacting the plant with a pesticidally effective amount of compounds of formula I. As such, “contacting” includes both direct contact (applying the compounds/compositions directly on the pest and/or plant—typically to the foliage, stem or roots of the plant) and indirect contact (applying the compounds/compositions to the locus of the pest and/or plant).

The aforementioned compositions are particularly useful for protecting crop plants against infestation of said pests or for combating these pests in infested plants.

For use in treating crop plants, the rate of application of the active ingredients of this invention may be in the range of 0.1 g to 4000 g per hectare, desirably from 25 g to 600 g per hectare, more desirably from 50 g to 500 g per hectare.

In the case of soil treatment or of application to the pests dwelling place or nest, the quantity of active ingredient ranges from 0.0001 to 500 g per 100 m², preferably from 0.001 to 20 g per 100 m².

The compounds of formula I are also suitable for the treatment of seeds in order to protect the seed from insect pest, in particular from soil-living insect pests and the resulting plant's roots and shoots against soil pests and foliar insects.

The compounds of formula I are particularly useful for the protection of the seed from soil pests and the resulting plant's roots and shoots against soil pests and foliar insects. The protection of the resulting plant's roots and shoots is preferred. More pre-ferred is the protection of resulting plant's shoots from piercing and sucking insects, wherein the protection from aphids is most preferred.

The present invention therefore comprises a method for the protection of seeds from insects, in particular from soil insects and of the seedlings' roots and shoots from insects, in particular from soil and foliar insects, said method comprising contacting the seeds before sowing and/or after pregermination with a compound of the general formula I or a salt thereof. Particularly preferred is a method, wherein the plant's roots and shoots are protected, more preferably a method, wherein the plants shoots are protected form piercing and sucking insects, most preferably aa method, wherein the plants shoots are protected from aphids.

The term seed embraces seeds and plant propagules of all kinds including but not limited to true seeds, seed pieces, suckers, corms, bulbs, fruit, tubers, grains, cuttings, cut shoots and the like and means in a preferred embodiment true seeds.

Compositions which are useful for seed treatment are e.g.:

A Soluble concentrates (SL, LS)

### D Emulsions (EW, EO, ES)

### E Suspensions (SC, OD, FS)

F Water-dispersible granules and water-soluble granules (WG, SG)
G Water-dispersible powders and water-soluble powders (WP, SP, WS)
H Dustable powders (DP, DS)

Preferred FS formulations of compounds of formula I for seed treatment usually comprise from 0.1 to 80% by weight (1 to 800 g/L) of the active ingredient, from 0.1 to 20% by weight (1 to 200 g/L) of at least one surfactant, e.g. 0.05 to 5% by weight of a wetter and from 0.5 to 15% by weight of a dispersing agent, up to 20% by weight, e.g. from 5 to 20% of an anti-freeze agent, from 0 to 15% by weight, e.g. 1 to 15% by weight of a pigment and/or a dye, from 0 to 40% by weight, e.g. 1 to 40% by weight of a binder (sticker/adhesion agent), optionally up to 5% by weight, e.g. from 0.1 to 5% by weight of a thickener, optionally from 0.1 to 2% of an anti-foam agent, and optionally a preservative such as a biocide, antioxidant or the like, e.g. in an amount from 0.01 to 1% by weight and a filler/vehicle up to 100% by weight.

Suitable pigments or dyes for seed treatment formulations are pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Binders, which are also referred to as stickers/adhesion agents are added to improve the adhesion of the active materials on the seeds after treatment. Suitable adhesives are block copolymers EO/PO surfactants but also polyvinylalcohols, polyvinylpyrrolidones, polyacrylates, polymethacrylates, polybutenes, polyisobutylenes, polystyrene, polyethyleneamines, polyethyleneamides, polyethyleneimines (Lupasol®, Polymin®), polyethers and copolymers derived from these polymers.

In the treatment of seed, the application rates of the compounds I are generally from 0.1 g to 10 kg per 100 kg of seed, preferably from 1 g to 5 kg per 100 kg of seed, in particular from 1 g to 1000 g per 100 kg of seed.

The invention therefore also relates to seed comprising a compound of the formula I or an agriculturally useful salt of I, as defined herein. The amount of the compound I or the agriculturally useful salt thereof will in general vary from 0.1 g to 10 kg per 100 kg of seed, preferably from 1 g to 5 kg per 100 kg of seed, in particular from 1 g to 1000 g per 100 kg of seed.

The compounds of the invention may also be applied against non-crop insect pests, such as ants, termites, wasps, flies, mosquitos, crickets, or cockroaches. For use against said non-crop pests, compounds of formula I are preferably used in a bait composition.

The bait can be a liquid, a solid or a semisolid preparation (e.g. a gel). Solid baits can be formed into various shapes and forms suitable to the respective application e.g. granules, blocks, sticks, disks. Liquid baits can be filled into various devices to ensure proper application, e.g. open containers, spray devices, droplet sources, or evaporation sources. Gels can be based on aqueous or oily matrices and can be formulated to particular necessities in terms of stickyness, moisture retention or aging characteristics.

The bait employed in the composition is a product which is sufficiently attractive to incite insects such as ants, termites, wasps, flies, mosquitos, crickets etc. or cockroaches to eat it. The attractiveness can be manipulated by using feeding stimulants or sex pheromones. Food stimulants are chosen, for example, but not exclusively, from animal and/or plant proteins (meat-, fish- or blood meal, insect parts, egg yolk), from fats and oils of animal and/or plant origin, or mono-, oligo- or polyorganosaccharides, especially from sucrose, lactose, fructose, dextrose, glucose, starch, pectin or even molasses or honey. Fresh or decaying parts of fruits, crops, plants, animals, insects or specific parts thereof can also serve as a feeding stimulant. Sex pheromones are known to be more insect specific. Specific pheromones are described in the literature and are known to those skilled in the art.

For use in bait compositions, the typical content of active ingredient is from 0.001 weight % to 15 weight %, desirably from 0.001 weight % to 5% weight % of active compound.

Formulations of compounds of formula I as aerosols (e.g. in spray cans), oil sprays or pump sprays are highly suitable for the non-professional user for controlling pests such as flies, fleas, ticks, mosquitos or cockroaches. Aerosol recipes are preferably composed of the active compound, solvents such as lower alcohols (e.g. methanol, ethanol, propanol, butanol), ketones (e.g. acetone, methyl ethyl ketone), paraffin hydrocarbons (e.g. kerosenes) having boiling ranges of approximately 50 to 250° C., dimethylformamide, N-methylpyrrolidone, dimethyl sulfoxide, aromatic hydrocarbons such as toluene, xylene, water, furthermore auxiliaries such as emulsifiers such as sorbitol monooleate, oleyl ethoxylate having 3-7 mol of ethylene oxide, fatty alcohol ethoxylate, perfume oils such as ethereal oils, esters of medium fatty acids with lower alcohols, aromatic carbonyl compounds, if appropriate stabilizers such as sodium benzoate, amphoteric surfactants, lower epoxides, triethyl orthoformate and, if required, propellants such as propane, butane, nitrogen, compressed air, dimethyl ether, carbon dioxide, nitrous oxide, or mixtures of these gases.

The oil spray formulations differ from the aerosol recipes in that no propellants are used.

For use in spray compositions, the content of active ingredient is from 0.001 to 80 weights %, preferably from 0.01 to 50 weight % and most preferably from 0.01 to 15 weight %.

The compounds of formula I and its respective compositions can also be used in mosquito and fumigating coils, smoke cartridges, vaporizer plates or long-term vaporizers and also in moth papers, moth pads or other heat-independent vaporizer systems.

The present invention is now illustrated in further details by the following examples.

### SYNTHESIS EXAMPLES

### Example 2

### N-Acetyl-N-ethyl-2-cyano-3-methyl-benzene-sulfonamide

1.0 g (4.6 mmol) of N-Ethyl-2-cyano-3-methyl-benzene-sulfonamide was dissolved in 50 ml of acetonitrile and 0.91 g (8.9 mmol) of acetic acid anhydride and 50 mg of (0.45 mmol) of 4-DMAP (4-dimethylaminopyridine) were added. The solution was stirred overnight at room temperature. After concentrating the reaction mixture water and methyl tert-butyl ether were added. The layers were separated and the aqueous layer was extracted three times with methyl tert-butyl ether. The combined organic layers were washed with phosphate buffer, dried over a drying agent and filtered off with suction. The filtrate was concentrated in vacuo to afford 1.07 g (90% of theory) of N-Acetyl-N-Ethyl-2-cyano-3-methyl-benzene-sulfonamide.

### Example 18

### 2-Methyl-6-(2-oxo-pyrrolidine-1-sulfonyl)benzonitrile

0.79 g (9.3 mmol) of 2-pyrrolidin-2-one were dissolved in 20 ml of dry tetrahydrofuran (THF) and cooled to −78° C. under inert atmosphere. 4.4 ml (7.0 mmol) of n-butyllithium were added slowly. Stirring was continued for 15 min at −78° C. and for 30 min at 0° C. The solution was slowly added to a solution of 1.00 g (4.6 mmol) of 2-cyano-3-methylbenzenesulfonylchloride in 20 ml of dry THF and stirring was continued until consumption of the sulfonyl chloride could be observed. The mixture was diluted by adding methyl tert-butyl ether and cooled to 0° C. Aqueous NH₄Cl was added, the layers were separated and the aqueous layer was extracted two times with methyl tert-butyl ether. The combined organic layers were washed with brine, dried over a drying agent and filtered off with suction. The filtrate was concentrated in vacuo and purified by flash chromatography (cyclohexane/ethyl acetate 4:1) to afford 0.10 g (8% of theory) of 2-methyl-6-(2-oxo-pyrrolidine-1-sulfonyl)benzonitrile.

### Example 20

### N-(2,2-Dimethylpropionoyl)-N-methyl-2-cyano-3-methyl-benzenesulfonamide

42 mg (1.05 mmol) of NaH (60% in mineral oil) were suspended in 10 ml of dry THF under an inert atmosphere. 0.21 g (1.0 mmol) of N-methyl-2-cyano-3-methyl-benzenesulfonamide in 10 ml of dry THF were added slowly at room temperature. Stirring was continued for 30 min at room temperature before 0.12 g (1.0 mmol) of pivaloyl chloride were slowly added and stirring was continued overnight at room temperature. The mixture was diluted with dichloromethane and aqueous ammonium chloride. The layers were separated and the aqueous layer was extracted three times with dichloromethane. The combined organic layers were washed water, dried over a drying agent and filtered off with suction. The filtrate was concentrated in vacuo and purified by flash chromatography (cyclohexane/ethyl acetate) to afford 0.27 g (92% of theory) of N-(2,2-dimethylpropionoyl-N-methyl-2-cyano-3-methyl-benzene-sulfonamide.

### Example 22

### N-Ethyl-N-(2-cyano-3-methyl-benzenesulfonyl)-N′-phenyl urea

1.0 g (4.5 mmol) of N-ethyl-2-cyano-3-methyl-benzene-sulfonamide was dissolved in 25 ml of dichloromethane followed by the addition of 0.8 g of phenylisocyanate and 0.6 g of N-ethyldiisopropylamine. The solution was stirred overnight at room temperature. Aqueous HCl was added, the layers were separated and the aqueous layer was extracted three times with dichloromethane. The combined organic layers were washed with water, dried over a drying agent and filtered off with suction. The filtrate was concentrated in vacuo and purified by flash chromatography (toluene/ethyl acetate) to afford 0.97 g (63% of theory) of N-Ethyl-N-(2-cyano-3-methyl-benzenesulfonyl)-N′-phenyl urea.

### Example 28

### N-(Benzyloxycarbonyl)-2-cyano-3-methyl-benzenesulfonamide

1.00 g (5.1 mmol) of 2-cyano-3-methyl-benzenesulfonamide was dissolved in 25 ml of acetone and cooled to 0° C. 0.20 g of NaOH (5.1 mmol) were added, followed by adding 0.87 g (5.1 mmol) of benzylchloroformate. The mixture was stirred at 0-5° C. for 1 h. The mixture was concentrated, diluted with water and acidified by the addition of 10% HCl. The solution was extracted three times with dichloromethane. The combined organic extracts were dried over a drying agent and filtered off with suction. The filtrate was concentrated in vacuo and purified by flash chromatography (cyclohexane/ethyl acetate) to afford 1.03 g (61% of theory) of N-(benzyloxycarbonyl)-2-cyano-3-methylbenzenesulfonamide.

### Example 29

### N-(Isopropylamino-ethoxy-methylene)-2-cyano-3-methyl-benzenesulfonamide

0.50 g (1.7 mmol) of 2-cyano-N-diethoxymethylene-3-methyl-benzenesulfonamide and 1.00 g (16.9 mmol) of isopropylamine were mixed and heated at 70° C. for 4 h. The mixture was concentrated and the residue was purified by flash chromatography on silica (cyclohexane/ethyl acetate) to yield 220 mg (42% of theory) of N-(isopropyl)aminoethoxy-methylene)-2-cyano-3-methyl-benzenesulfonamide having a melting point of 139-142° C.

### Example 32

### 2-Cyano-N-dimethoxymethylene-3-methyl-benzenesulfonamide

2.00 g (10.2 mmol) of 2-cyano-3-methyl-benzenesulfonamide and 2.08 g of tetramethoxymethane (15.3 mmol) were heated to 160° C. slowly and under stirring and under removal of methanol from the mixture. The mixture was cooled to room temperature. The residue was washed with methyl t-butyl ether and dried to afford 1.71 g (63% of theory) of 2-cyano-N-dimethoxymethylene-3-methyl-benzenesulfonamide.

### Example 34

### 2-Cyano-N-(1-methoxyethylidene)-3-methyl-benzenesulfonamide

1.00 g (5.1 mmol) of 2-cyano-3-methyl-benzenesulfonamide and 6.12 g of trimethoxyethane (51.0 mmol) were heated to reflux for 3 h. The mixture was cooled to room temperature. Excess of trimethoxyethane was removed by distillation. Cyclohexane was added. The precipitate was sucked off and dried to afford 1.12 g (87% of theory) of 2-cyano-N-(1-methoxyethylidene)-3-methyl-benzenesulfonamide.

### Example 35

### 2-Cyano-3-methyl-N-dimethylaminomethylene-benzenesulfonamide

2.00 g (10.2 mmol) of 2-cyano-3-methyl-benzenesulfonamide and 1.82 g of dimethylformamide dimethyl acetal in 25 ml of toluene were heated to reflux for 2 h. The mixture was cooled to room temperature. The brownish precipitate was sucked off and purified by chromatography to yield 0.66 g (26% of theory) of 2-cyano-3-methyl-N-dimethylaminomethylene-benzenesulfonamide having a melting point of 168-174° C.

### Example 37

### 2-Cyano-3-methyl-N-(1,2,2-trimethyl-propylidene)-benzenesulfonamide

0.70 g (3.6 mmol) of 2-cyano-3-methyl-benzenesulfonamide and 0.36 g (3.6 mmol) of 3,3-dimethyl-butan-2-one were mixed in 15 ml of 1,1,2-trichloroethane and cooled to 0° C. 0.68 g of titaniumtetrachloride, dissolved in 5 ml of dry dichloromethane, was added slowly. The reaction mixture was heated to reflux for 14 h. The reaction mixture was concentrated, the residue was dissolved in dichloromethane and washed 3 times with 5% K₂CO₃-solution. The organic layer was dried and concentrated. The residue was purified by flash chromatography on silica (cyclohexane/ethyl acetate 4:1) to yield 45 mg (5% of theory) of 2-cyano-3-methyl-N-(1,2,2-trimethyl-propylidene)-benzenesulfonamide.

### Examples 38

### 2-Cyano-3-methyl-N-(tetrahydro-thiphophen-1-ylidene)benzenesulfonamide

To a solution of 0.44 g (5.00 mmol) of tetrahydrothiophene in 10 ml of methanol were added 0.57 g tert-butylhypochlorite at −78° C. After 20 min, 0.7 ml of triethylamine and 0.98 g of 2-cyano-3-methylbenzenesulfonamide dissolved in 5 ml of methanol and 3 ml of DMF were added. Stirring was continued at −78° for 2 h and overnight at room temperature. 5% NaOH-solution was added, the layers were separated and the aqueous layer was extracted two times with dichloromethane. The combined organic extracts were dried over a drying agent and filtered off with suction. The filtrate was concentrated in vacuo and purified by chromatography on silica (toluene/ethanol 4:1) to afford 0.153 g (11% of theory) of the title compound.

### Example 41

### 2-Methyl-6-(pyrrole-1-sulfonyl)-benzonitrile

To a solution of 1.00 g (5.10 mmol) of 2-cyano-3-methyl-benzenesulfonamide in 25 ml of acetic acid was added 1.01 g (7.64 mmol) of 2,5-dimethoxy-tetrahydrofuran at room temperature while stirring. After completion of addition, the solution was heated to reflux for 1 hour. After cooling to room temperature water was added, and the solution was extracted with ethyl acetate. The combined organic extracts were dried over a drying agent and filtered off with suction. The filtrate was concentrated in vacuo and purified by chromatography on silica (cyclohexane/ethyl acetate 4:1) to afford 0.64 g (51% of theory) of 2-methyl-6-(pyrrole-1-sulfonyl)-benzonitrile.

### Example 45

### 2-Methyl-6-(3-bromo-pyrrole-1-sulfonyl)-benzonitrile

1.0 g (4.1 mmol) of 2-methyl-6-(pyrrole-1-sulfonyl)-benzonitrile were suspended in 15 ml of glacial acetic acid at room temperature. 0.22 ml (4.3 mmol) of bromine were dissolved in 15 ml of acetic acid and added slowly at room temperature during 30 min. The mixture was heated to reflux for 1.5 h and stirred overnight at room temperature. The mixture was poured on ice. The precipitate was sucked off, dissolved in ethyl acetate, dried over a drying agent and concentrated in vacuo. The residue was repetitively purified by chromatography on silica to afford 81 mg (6% of theory) of 2-methyl-6-(3-bromo-pyrrole-1-sulfonyl)-benzonitrile having a melting point of 98-100° C.

### Example 46

### 2-Methyl-6-(pyrazole-1-sulfonyl)-benzonitrile

To a solution of 0.41 g (1.90 mmol) of 2-cyano-3-methyl-benzenesulfonylchloride in 10 ml of dichloromethane was added 0.19 g (2.81 mmol) of pyrazole and 0.39 g of triethylamine at room temperature. After stirring overnight, TLC showed completion of the reaction. Ammonium chloride solution was added, the layers were separated and the aqueous layer was extracted three times with dichloromethane. The combined organic extracts were dried over a drying agent and filtered off with suction. The filtrate was concentrated in vacuo and purified by chromatography on silica (cyclohexane/ethyl acetate 1:1) to afford 0.184 g (40% of theory) of 2-methyl-6-(pyrazole-1-sulfonyl)benzonitrile having a melting point of 99-104° C.

### Example 47

### 2-Methyl-6-([1.2.4]-triazole-1-sulfonyl)-benzonitrile

To a solution of 0.41 g (1.90 mmol) of 2-cyano-3-methyl-benzenesulfonylchloride in 10 ml of dichloromethane was added 0.19 g (2.79 mmol) of [1.2.4]-triazole and 0.39 g triethylamine at room temperature. After stirring overnight, TLC showed completion of the reaction. Ammonium chloride solution was added, the layers were separated and the aqueous layer was extracted three times with dichloromethane. The combined organic extracts were dried over a drying agent and filtered off with suction. The filtrate was concentrated in vacuo and purified by chromatography on silica (cyclohexane/ethyl acetate 1:1) to afford 0.108 g (23% of theory) of 2-methyl-6-([1.2.4]-triazole-1-sulfonyl)benzonitrile having a melting point of 142-144° C.

### Example 48

### 2-Methyl-6-(imidazole-1-sulfonyl)-benzonitrile

To a solution of 0.41 g (1.90 mmol) of 2-cyano-3-methyl-benzenesulfonylchloride in 10 ml of dichloromethane was added 0.19 g (2.81 mmol) of imidazole and 0.39 g of triethylamine at room temperature. After stirring overnight, TLC showed completion of the reaction. Ammonium chloride solution was added, the layers were separated and the aqueous layer was extracted three times with dichloromethane. The combined organic extracts were dried over a drying agent and filtered off with suction. The filtrate was concentrated in vacuo and purified by chromatography on silica (cyclohexane/ethyl acetate 1:1) to afford 0.126 g (27% of theory) of 2-methyl-6-(imidazole-1-sulfonyl)benzonitrile having a melting point of 93-95° C.

The compounds of the formula I with m=2, R²=H, R³=H, R⁴=H or F listed in the following table I were prepared in an analogous manner.

TABLE I

| | | m.p. [° C.]¹⁾ or | |
|---|---|---|---|
| Example | structure | chemical shift²⁾ | RT (HPLC/MS)³⁾ |
| 1 | 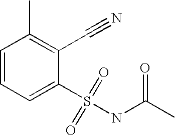 | 116-118 | |
| 2 | 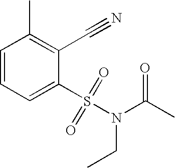 | ¹H-NMR (DMSO-d₆):1.33 (t, 3 H), 2.27 (s, 3 H),2.58 (s, 3 H), 3.98 (q, 2 H),7.85 (m, 2 H), 8.03(d, 1 H). | |
| 3 | 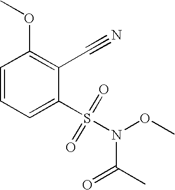 | 109-112 | |
| 4 | 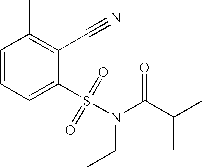 | ¹H-NMR (DMSO-d₆):0.97 (d, 6 H), 1.31 (t, 3 H),2.56 (s, 3 H), 3.02 (m,1 H), 3.98 (q, 2 H), 7.83(m, 2 H), 8.02 (d, 1 H). | |
| 5 | 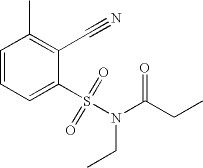 | | tᵣ = 2.86 min,m/z = 281 [m + 1] |
| 6 | 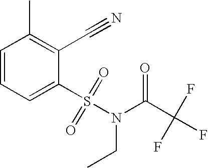 | ¹H-NMR (DMSO-d₆):1.02 (t, 3 H), 2.57 (s, 3 H),2.95 (m, 2 H), 7.78 (m,2 H), 8.02 (t, 1 H) | |
| 7 | 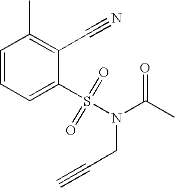 | ¹H-NMR (DMSO-d₆):2.32 (s, 3 H), 2.58 (s, 3 H),3.49 (s, 1 H), 4.73 (d, 2 H),7.86 (m, 2 H), 8.04 (t, 1 H). | |
| 8 | 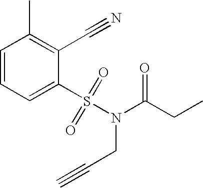 | ¹H-NMR (DMSO-d₆):0.93 (t, 3 H), 2.58 (s, 3 H),2.70 (q, 2 H), 3.51 (s, 1 H),4.76 (d, 2 H), 7.86 (m,2 H), 8.05 (m, 1 H). | |
| 9 | 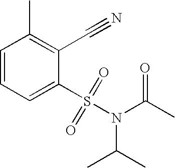 | ¹H-NMR (DMSO-d₆):1.36 (d, 6 H), 2.37 (s, 3 H),2.62 (s, 3 H), 4.24 (sept.,1 H), 7.87 (m, 2 H), 8.06(m, 1 H). | |
| 10 | 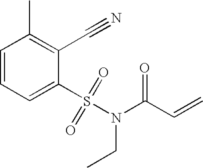 | | tᵣ = 2.97 min,m/z = 301[m + 23] |
| 11 | 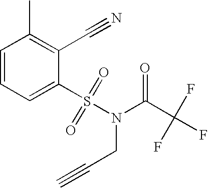 | ¹H-NMR (DMSO-d₆):2.57 (s, 3 H), 3.05 (s, 1 H),3.84 (s, 2 H), 7.77 (m,2 H), 7.87 (d, 1 H). | |
| 12 | 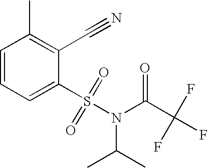 | ¹H-NMR (DMSO-d₆):1.35 (d, 6 H), 2.57 (s, 3 H),3.40 (m, 1 H), 7.78 (m,2 H), 7.88 (d, 1 H). | |
| 13 | 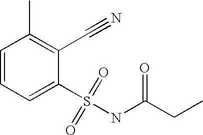 | ¹H-NMR (DMSO-d₆):0.91 (t, 3 H), 2.28 (q, 2 H),2.58 (s, 3 H), 7.82 (m,2 H), 7.99 (d, 1 H) | |
| 14 | 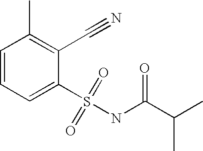 | ¹H-NMR (DMSO-d₆):0.98 (d, 6 H), 2.58 (s, 3 H),5.75 (s, 1 H), 7.82 (m,2 H), 7.98 (d, 1 H). | |
| 15 | 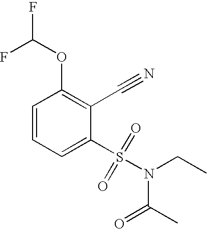 | 129-131 | |
| 16 | 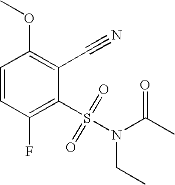 | ¹H-NMR (CDCl₃):1.35 (t, 3 H), 2.5 (s, 3 H),3.90 (q, 2 H), 4.00 (s, 3 H),7.20 (m, 1 H), 7.45 (m,1 H). | |
| 17 | 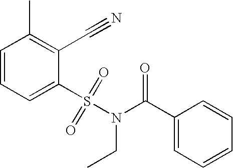 | | tᵣ = 3.39 min,m/z = 329 [m + 1] |
| 18 | 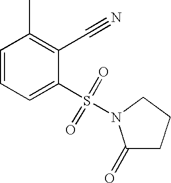 | | tᵣ = 2.38 min,m/z = 265 [m + 1] |
| 19 | 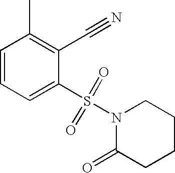 | | tᵣ = 2.55 min,m/z = 279 [m + 1] |
| 20 | 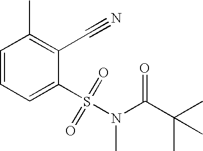 | | tᵣ = 2.92 min,m/z = 295 [m + 1] |
| 21 | 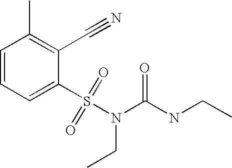 | 108-110 | |
| 22 | 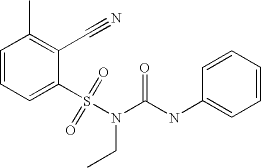 | | tᵣ = 3.54 min,m/z = 344 [m + 1] |
| 23 | 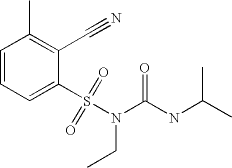 | | tᵣ = 3.18 min,m/z = 310 [m + 1] |
| 24 | 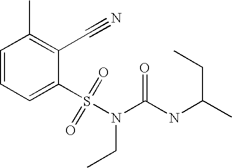 | | tᵣ = 3.33 min,m/z = 324 [m + 1] |
| 25 | 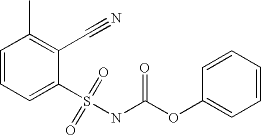 | ¹H-NMR (DMSO-d₆):6.93 (m, 2 H), 7.05 (t, 1 H),7.25 (m, 2 H), 7.50 (d,1 H), 7.58 (t, 1 H), 7.78 (d,1 H). | |
| 26 | 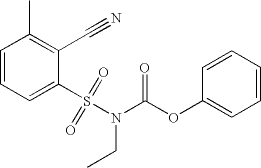 | ¹H-NMR (DMSO-d₆):1.42 (t, 3 H), 2.61 (s, 3 H),4.12 (q, 2 H), 6.98 (m,2 H), 7.26 (m, 1 H), 7.38(m, 1 H), 7.48 (m, 1 H),7.83 (m, 2 H), 8.04 (d,1 H). | |
| 27 | 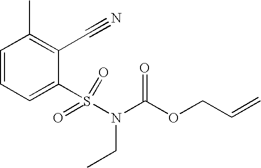 | ¹H-NMR (DMSO-d₆):1.32 (t, 3 H), 2.57 (s, 3 H),3.97 (q, 2 H), 4.55 (d, 2 H),5.13 (d, 1 H), 5.17 (d, 1 H),5.73 (m, 1 H), 7.85 (m,2 H), 8.03 (t, 1 H). | |
| 28 | 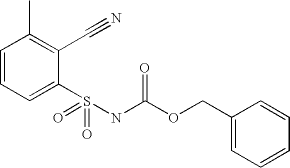 | | tᵣ = 3.06 min,m/z = 353[m + 23] |
| 29 | 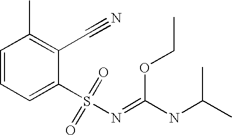 | 139-142 | |
| 30 | 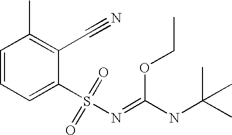 | 160-165 | |
| 31 | 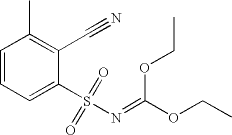 | ¹H-NMR (DMSO-d₆):1.18 (t, 6 H), 2.57 (s, 3 H),4.33 (q, 4 H), 7.78 (m,2 H), 7.90 (d, 1 H). | |
| 32 | 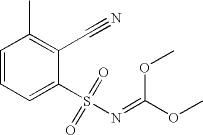 | ¹H-NMR (DMSO-d₆):2.60 (s, 3 H), 3.41 (s, 3 H),3.63 (s, 3 H), 7.85 (m,2 H), 8.07 (t, 1 H). | |
| 33 | 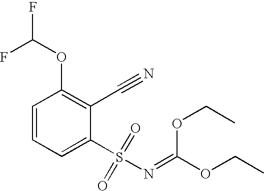 | | tᵣ = 2.89 min,m/z = 349 [m + 1] |
| 34 | 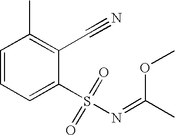 | | tᵣ = 2.64 min,m/z = 253 [m + 1] |
| 35 | 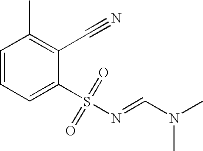 | 168-174 | |
| 36 | 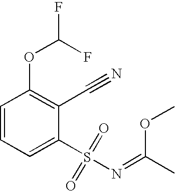 | 78-82 | |
| 37 | 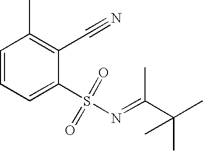 | | tᵣ = 2.67 min,m/z = 279 [m + 1] |
| 38 | 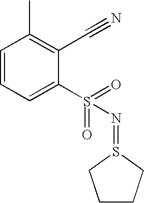 | | tᵣ = 1.95 min,m/z = 283 [m + 1] |
| 39 | 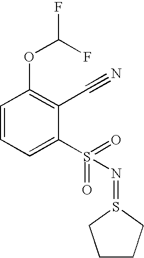 | | tᵣ = 2.00 min,m/z = 335 [m + 1] |
| 40 | 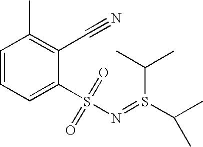 | ¹H-NMR (CDCl₃):1.24 (d, 6 H), 1.47 (d, 6 H),2.61 (s, 3 H), 7.41 (d, 1 H),7.52 (t, 1 H), 7.96 (m, 1 H) | |
| 41 | 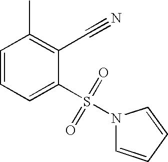 | ¹H-NMR (DMSO-d₆):2.57 (s, 3 H), 6.45 (s, 2 H),7.37 (m, 2 H), 7.90(m, 2 H), 7.97 (m, 1 H). | |
| 42 | 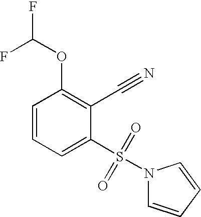 | 75-80 | |
| 43 | 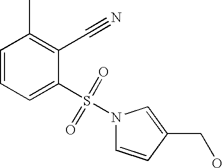 | ¹H-NMR (CDCl₃):2.65 (s, 3 H), 4.93 (s, 2 H),6.35 (s, 1 H), 7.29 (s, 2 H),7.62 (m, 2 H), 7.98 (d,1 H). | |
| 44 | 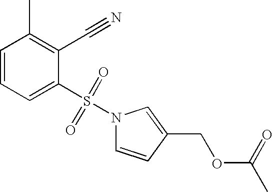 | 109-111 | |
| 45 | 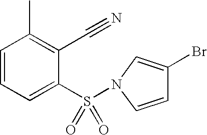 | 97-101 | |
| 46 | 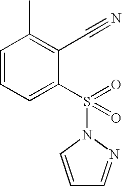 | 99-104 | |
| 47 | 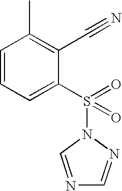 | 142-144 | |
| 48 | 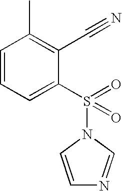 | 93-95 | |

The products were characterized by coupled High Performance Liquid Chromatography/mass spectrometry (HPLC/MS), by NMR or by their melting points.
¹⁾m.p. melting point
²⁾¹H-NMR: The signals are characterized by chemical shift (ppm) vs. tetramethylsilane, by their multiplicity and by their integral (relative number of hydrogen atoms given). The following abbreviations are used to characterize the multiplicity of the signals: m = multiplett, q = quartett, t = triplett, d = doublet and s = singulett.
³⁾RT = tᵣ retention time Analytical HPLC column: RP-18 column Chromolith Speed ROD (Merck KgaA, Germany). Elution: acetonitrile + 0.1% trifluoroacetic acid/water + 0.1% trifluoroacetic acid in a ratio of from 5:95 to 95:5 in 5 min at 40° C.

### II. Examples of Action Against Pests

The active compounds were formulated in a mixture of 50 vol.-% acetone:50 vol.-% water. A nonionic surfactant (Kinetic®) was included in the solution at a volume of 0.01% v/v.

In the following tests, the formulated solutions of the active compounds were diluted to an active ingredient concentration of 300 ppm and the diluted solutions were applied in the below mentioned tests.

The action of the compounds of the formula I against pests was demonstrated by the following experiments:

II.1 Cotton Aphid (aphis gossypii), Mixed Life Stages

Cotton plants at the cotyledon stage were infested prior to treatment by placing a heavily infested leaf from the main aphid colony on top of each cotyledon. The aphids were allowed to transfer overnight and the host leaf was removed. The infested cotyledons were then dipped and agitated in the test solution for 3 seconds and allowed to dry in a fume hood. Test plants were maintained under fluorescent lighting in a 24-hr photoperiod at 25° C. and 20-40% relative humidity. Aphid mortality on the treated plants, relative to mortality on untreated check plants, was determined after 5 days.

In this test compounds each of examples 1, 2, 3, 5, 6, 7, 8, 9, 10, 11, 12, 13, 14, 15, 16, 17, 18, 19, 21, 22, 23, 24, 25, 26, 27, 28, 31, 32, 33, 34, 35, 36, 37, 38, 39, 40, 41, 42, 43, 44, 45, 48 at 300 ppm provided at least 86% mortality of cotton aphid (Aphis gossypii, mixed life stages) in comparison with untreated controls.

II.2 Green Peach Aphid (Myzus persicae), Mixed Life Stages

Bell pepper plants at the first true-leaf stage were infested prior to treatment by placing heavily infested leaves from the main aphid colony on top of the treatment plants. The aphids were allowed to transfer overnight to accomplish an infestation of 30-40 aphids per plant and the host leaves were removed. The infested leaves of the test plants were then dipped and agitated in the test solution for 3 seconds and allowed to dry in a fume hood. Test plants were maintained under fluorescent lighting in a 24-hr photoperiod at 25° C. and 20-40% relative humidity. Aphid mortality on the treated plants, relative to mortality on untreated check plants, was determined after 5 days.

In this test compounds each of examples 1, 2, 3, 5, 6, 7, 8, 9, 11, 12, 13, 14, 15, 16, 21, 22, 23, 27, 28, 31, 33, 34, 35, 36, 37, 38, 39, 40, 41, 42, 43, 44, 45, 48 at 300 ppm provided at least 86% mortality of green peach aphidin comparison with untreated controls.

11.3 Bean Aphid (Aphis fabae)

Nasturtium plants grown in Metro mix in the 1^{st} leaf-pair stage (variety ‘Mixed Jewel’) were infested with approximately 2-30 laboratory-reared aphids by placing infested cut plants on top of the test plants. The cut plants were removed after 24 hr. Each plant was dipped into the test solution to provide complete coverage of the foliage, stem, protruding seed surface and surrounding cube surface and allowed to dry in the fume hood. The treated plants were kept at about 25° C. with continuous fluorescent light. Aphid mortality is determined after 3 days.

In this test compounds each of examples 2, 5, 6, 7, 8, 11, 12, 13, 14, 15, 17, 18, 21, 23, 25, 26, 27, 30, 31, 32, 33, 35, 36, 37, 38, 41, 42, 44 at 300 ppm provided at least 86% mortality of bean aphid in comparison with untreated controls.

We claim:

1-28. (canceled)

29. A method of combating animal pests comprising,
contacting the animal pests, their habit, breeding ground, food supply, plant, seed, soil, area, material or environment in which the animal pests are growing or may grow, or the materials, plants, seeds, soils, surfaces or spaces to be protected from animal attack or infestation with a pesticidally effective amount of at least one cyanobenzene compound of the formula I and/or at least one agriculturally acceptable salt thereof: 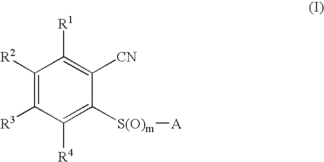
wherein
m is 0, 1 or 2;
A is a radical of the formulae
N═CR⁵R⁶, N═SR⁷R⁸, NR¹⁰—C(═X)—R⁹,
where X is O or S,
or A is a N-bound 5-, 6- or 7-membered heterocycle, which is ethylenically unsaturated or aromatic, and which additionally may contain 1, 2, or 3 further heteroatoms or heteroatom groups, selected from O, S, SO, SO₂, N, and NR¹², and/or 1, 2 or 3 carbonyl groups as ring members and which may carry 1, 2, 3 or 4 radicals R¹³,
R¹ is hydrogen, nitro, cyano, azido, amino, halogen, sulfenylamino, sulfinylamino, sulfonylamino, C(═O)R¹⁴, C₁-C₆-alkyl, C₂-C₆-alkenyl, C₂-C₆-alkynyl, C₃-C₈-cycloalkyl, C₁-C₆-alkoxy, (C₁-C₆-alkyl)amino, di(C₁-C₆-alkyl)amino, C₁-C₆-alkylthio, C₁-C₆-alkylsulfinyl or C₁-C₆-alkylsulfonyl, wherein the ten last-mentioned radicals may be unsubstituted, partially or fully halogenated and/or may carry 1, 2 or 3 radicals, selected from the group consisting of cyano, nitro, amino, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, (C₁-C₄-alkoxy)carbonyl, (C₁-C₄-alkyl)amino, di(C₁-C₄-alkyl)amino, C₃-C₈-cycloalkyl and phenyl, it being possible for phenyl to be unsubstituted, partially or fully halogenated and/or to carry 1, 2 or 3 substituents, independently of one another selected from the group consisting of C₁-C₄-alkyl, C₁-C₄-haloalkyl, C₁-C₄-alkoxy and C₁-C₄-haloalkoxy;
R², R³ and R⁴ are independently of one another selected from the group consisting of hydrogen, halogen, cyano, azido, nitro, C₁-C₆-alkyl, C₃-C₈-cycloalkyl, C₁-C₄-haloalkyl, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, C₂-C₆-alkenyl, C₂-C₆-alkynyl, amino, (C₁-C₄-alkyl)amino, di(C₁-C₄-alkyl)amino, sulfonylamino, sulfinylamino, sulfenylamino and C(═O)—R¹⁵;
R⁵ is H, OR^{5a}, NR^{5b}R^{5c}, aryl, aryl-C₁-C₄-alkyl, heteroaryl, heteroaryl-C₁-C₄-alkyl, heterocyclyl, heterocyclyl-C₁-C₄-alkyl, C₁-C₁₀-alkyl, C₃-C₁₀-cycloalkyl, C₁-C₁₀-alkylthio, C₁-C₁₀-alkylsulfonyl or C₁-C₁₀-alkylsulfinyl, wherein the five last-mentioned radicals may be unsubstituted, partially or fully halogenated and/or may carry 1, 2 or 3 radicals, independently of one another each selected from the group consisting of cyano, nitro, amino, C₁-C₁₀-alkoxy, C₁-C₁₀-alkylthio, C₁-C₁₀-alkylsulfinyl, C₁-C₁₀-alkylsulfonyl, C₁-C₁₀-haloalkoxy, C₁-C₁₀-haloalkylthio, C₁-C₁₀-alkoxycarbonyl, (C₁-C₁₀-alkyl)amino, di-(C₁-C₁₀-alkyl)amino, C₃-C₁₀-cycloalkyl and phenyl, it being possible for phenyl to be unsubstituted, partially or fully halogenated and/or to carry 1, 2 or 3 substituents, independently of one another selected from the group consisting of C₁-C₄-alkyl, C₁-C₄-haloalkyl, C₁-C₄-alkoxy and C₁-C₄-haloalkoxy;
wherein
R^{5a} is C₁-C₁₀-alkyl, C₃-C₁₀-cycloalkyl, C₂-C₁₀-alkenyl, C₂-C₁₀-alkynyl, aryl, aryl-C₁-C₄-alkyl, heteroaryl or heteroaryl-C₁-C₄-alkyl, heterocyclyl or heterocyclyl-C₁-C₄-alkyl;
R^{5b}, R^{5c}, independently from each other, are hydrogen, C₁-C₁₀-alkyl, C₃-C₁₀-cycloalkyl, C₂-C₁₀-alkenyl, C₂-C₁₀-alkynyl, aryl, aryl-C₁-C₄-alkyl, heteroaryl and heteroaryl-C₁-C₄-alkyl, heterocyclyl or heterocyclyl-C₁-C₄-alkyl;
wherein C₁-C₁₀-alkyl, C₃-C₁₀-cycloalkyl, C₂-C₁₀-alkenyl and C₂-C₁₀-alkynyl in R^{5a}, R^{5b} and R^{5c} may be unsubstituted, partially or fully halogenated and/or may carry 1, 2 or 3 radicals, selected from the group consisting of cyano, nitro, amino, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, (C₁-C₄-alkoxy)carbonyl, (C₁-C₄-alkyl)amino, di(C₁-C₄-alkyl)amino and C₃-C₈-cycloalkyl;
wherein the heteroaryl moiety in heteroaryl and heteroaryl-C₁-C₄-alkyl of R⁵, R^{5a}, R^{5b} and R^{5c} is 5- or 6 membered and contains 1, 2, 3 or 4 heteroatoms and/or heteroatom groups, selected from the group consisting of O, S, SO, SO₂, N and NR^{a}, as ring members, R^{a} being hydrogen or C₁-C₆-alkyl;
wherein the heterocyclyl moiety in heterocyclyl and heterocyclyl-C₁-C₄-alkyl of R⁵, R^{5a}, R^{5b} and R^{5c} is 3- to 7 membered, is saturated or partly unsaturated and contains 1, 2, 3 or 4 heteroatoms and/or heteroatom groups, selected from the group consisting of O, S, SO, SO₂, N and NR^{b}, as ring members, R^{b} being hydrogen or C₁-C₆-alkyl, and additionally may contain 1, 2 or 3 CO groups as ring members;
and wherein the carbon atoms of aryl, hetaryl, and heterocyclyl moieties in R⁵, R^{5a}, R^{5b} and R^{5c} may be unsubstituted or may carry 1, 2 or 3 substituents, independently of one another selected from the group consisting of halogen, C₁-C₄-alkyl, C₁-C₄-haloalkyl, C₁-C₄-alkoxy and C₁-C₄-haloalkoxy;
R⁶ independently has one of the meanings given for R⁵;
R⁷, R⁸, independently from each other, are selected from the group consisting of aryl, aryl-C₁-C₄-alkyl, heteroaryl, heteroaryl-C₁-C₄-alkyl, heterocyclyl, heterocyclyl-C₁-C₄-alkyl, C₁-C₁₀-alkyl, C₃-C₁₀-cycloalkyl, C₂-C₁₀-alkenyl and C₂-C₁₀-alkynyl, wherein the four last-mentioned radicals may be unsubstituted, partially or fully halogenated and/or may carry 1, 2 or 3 radicals, independently of one another each selected from the group consisting of cyano, nitro, amino, C₁-C₁₀-alkoxy, C₁-C₁₀-alkylthio, C₁-C₁₀-alkylsulfinyl, C₁-C₁₀-alkylsulfonyl, C₁-C₁₀-haloalkoxy, C₁-C₁₀-haloalkylthio, C₁-C₁₀-alkoxycarbonyl, (C₁-C₁₀-alkyl)amino, di-(C₁-C₁₀-alkyl)amino, C₃-C₁₀-cycloalkyl and phenyl, it being possible for phenyl to be unsubstituted, partially or fully halogenated and/or to carry 1, 2 or 3 substituents, independently of one another selected from the group consisting of C₁-C₄-alkyl, C₁-C₄-haloalkyl, C₁-C₄-alkoxy and C₁-C₄-haloalkoxy; or
R⁷ and R⁸ together with the sulfur atom they are bound to form a saturated or ethylenically unsaturated 5- to 10-membered ring, optionally substituted by 1, 2, 3 or 4 radicals selected from the group consisting of C₁-C₅-alkyl and halogen, wherein the ring may contain, in addition to the sulfur atom, 1, 2 or 3 heteroatoms and/or heteroatom-containing groups as ring members selected from the group consisting of nitrogen, oxygen, sulfur, CO, SO, SO₂ and N—R¹⁷;
wherein the heteroaryl moiety in heteroaryl and heteroaryl-C₁-C₄-alkyl of R⁷ and R⁸ is 5- or 6 membered and contains 1, 2, 3 or 4 heteroatoms and/or heteroatom groups, selected from the group consisting of O, S, SO, SO₂, N and NRC, as ring members, R^{c} being hydrogen or C₁-C₆-alkyl,
wherein the heterocyclyl moiety in heterocyclyl and heterocyclyl-C₁-C₄-alkyl of R⁷ and R⁸ is 3- to 7-membered, is saturated or partly unsaturated and contains 1, 2, 3 or 4 heteroatoms and/or heteroatom groups, selected from the group consisting of O, S, SO, SO₂, N and NR^{d}, as ring members, R^{d} being hydrogen or C₁-C₆-alkyl, and additionally may contain 1, 2 or 3 CO groups as ring members,
and wherein the carbon atoms of aryl, hetaryl, and heterocyclyl in R⁷ and R⁸ may be unsubstituted or may carry 1, 2 or 3 substituents, independently of one another selected from the group consisting of halogen, C₁-C₄-alkyl, C₁-C₄-haloalkyl, C₁-C₄-alkoxy and C₁-C₄-haloalkoxy;
R⁹ is selected from the group consisting of hydrogen, OR^{9a}, NR^{9b}R^{9c}, aryl, aryl-C₁-C₄-alkyl, heteroaryl, heteroaryl-C₁-C₄-alkyl, heterocyclyl, heterocyclyl-C₁-C₄-alkyl, C₁-C₁₀-alkyl, C₂-C₁₀-alkenyl, C₂-C₁₀-alkynyl and C₃-C₁₀-cycloalkyl, wherein the four last-mentioned radicals may be unsubstituted, partially or fully halogenated and/or may carry 1, 2 or 3 radicals, independently of one another each selected from the group consisting of cyano, nitro, amino, C₁-C₁₀-alkoxy, C₁-C₁₀-alkylthio, C₁-C₁₀-alkylsulfinyl, C₁-C₁₀-alkylsulfonyl, C₁-C₁₀-haloalkoxy, C₁-C₁₀-haloalkylthio, C₁-C₁₀-alkoxycarbonyl, (C₁-C₁₀-alkyl)amino, di-(C₁-C₁₀-alkyl)amino, C₃-C₁₀-cycloalkyl and phenyl, it being possible for phenyl to be unsubstituted, partially or fully halogenated and/or to carry 1, 2 or 3 substituents, independently of one another selected from the group consisting of C₁-C₄-alkyl, C₁-C₄-haloalkyl, C₁-C₄-alkoxy and C₁-C₄-haloalkoxy,
and wherein
R^{9a} is C₁-C₁₀-alkyl, C₃-C₁₀-cycloalkyl, C₂-C₁₀-alkenyl, C₂-C₁₀-alkynyl, aryl, aryl-C₁-C₄-alkyl, heteroaryl or heteroaryl-C₁-C₄-alkyl, heterocyclyl or heterocyclyl-C₁-C₄-alkyl,
R^{9b}, R^{9c}, independently from each other, are selected from the group consisting of hydrogen, C₁-C₁₀-alkyl, C₃-C₁₀-cycloalkyl, C₂-C₁₀-alkenyl, C₂-C₁₀-alkynyl, aryl, aryl-C₁-C₄-alkyl, heteroaryl and heteroaryl-C₁-C₄-alkyl, heterocyclyl and heterocyclyl-C₁-C₄-alkyl,
wherein C₁-C₁₀-alkyl, C₃-C₁₀-cycloalkyl, C₂-C₁₀-alkenyl and C₂-C₁₀-alkynyl in R^{9a}, R^{9b} and R^{9c} may be unsubstituted, partially or fully halogenated and/or may carry 1, 2 or 3 radicals, selected from the group consisting of cyano, nitro, amino, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, (C₁-C₄-alkoxy)carbonyl, (C₁-C₄-alkyl)amino, di(C₁-C₄-alkyl)amino and C₃-C₈-cycloalkyl,
wherein the heteroaryl moiety in heteroaryl and heteroaryl-C₁-C₄-alkyl of R⁹, R^{9a}, R^{9b} and R^{9c} is 5- or 6-membered and contains 1, 2, 3 or 4 heteroatoms and/or heteroatom groups, selected from the group consisting of O, S, SO, SO₂, N and NR^{e}, as ring members, R^{e} being hydrogen or C₁-C₆-alkyl,
wherein the heterocyclyl moiety in heterocyclyl and heterocyclyl-C₁-C₄-alkyl of R⁹, R^{9a}, R^{9b} and R^{9c} is 3- to 7-membered, is saturated or partly unsaturated and contains 1, 2, 3 or 4 heteroatoms and/or heteroatom groups, selected from the group consisting of O, S, SO, SO₂, N and NR^{f}, as ring members, R^{f} being hydrogen or C₁-C₆-alkyl, and additionally may contain 1, 2 or 3 CO groups as ring members,
and wherein the carbon atoms of aryl, hetaryl and heterocyclyl moieties in R⁹, R^{9a}, R^{9b} and R^{9c} may be unsubstituted or may carry 1, 2 or 3 substituents, independently of one another selected from the group consisting of halogen, C₁-C₄-alkyl, C₁-C₄-haloalkyl, C₁-C₄-alkoxy and C₁-C₄-haloalkoxy;
R¹⁰ is selected from the group consisting of hydrogen, C(═O)—R¹⁶, C₁-C₁₀-alkyl, C₂-C₁₀-alkenyl, C₂-C₁₀-alkynyl, C₁-C₁₀-alkoxy and C₃-C₁₀-cycloalkyl, wherein the five last-mentioned radicals may be unsubstituted, partially or fully halogenated and/or may carry 1, 2 or 3 radicals, independently of one another each selected from the group consisting of cyano, nitro, amino, C₁-C₁₀-alkoxy, C₁-C₁₀-alkylthio, C₁-C₁₀-alkylsulfinyl, C₁-C₁₀-alkylsulfonyl, C₁-C₁₀-haloalkoxy, C₁-C₁₀-haloalkylthio, C₁-C₁₀-alkoxycarbonyl, (C₁-C₁₀-alkyl)amino, di-(C₁-C₁₀-alkyl)amino, C₃-C₁₀-cycloalkyl and phenyl, it being possible for phenyl to be unsubstituted, partially or fully halogenated and/or to carry 1, 2 or 3 substituents, independently of one another selected from the group consisting of C₁-C₄-alkyl, C₁-C₄-haloalkyl, C₁-C₄-alkoxy and C₁-C₄-haloalkoxy; or
R⁹ and R¹⁰ together with the adjacent nitrogen and carbon atoms form a saturated or ethylenically unsaturated 5 to 10-membered ring, optionally substituted by 1, 2, 3 or 4 radicals selected from C₁-C₅-alkyl and halogen, wherein the ring may contain, in addition to the nitrogen and carbon ring members, 1, 2 or 3 heteroatoms and/or heteroatom groups as ring members selected from the group consisting of nitrogen, oxygen, sulfur, CO, SO, SO₂ and N—R¹⁷;
R¹² is hydrogen, C₁-C₆-alkyl, C₁-C₄-alkoxy-C₁-C₄-alkyl or C₁-C₆-alkoxy;
each R¹³ independently is selected from the group consisting of halogen, cyano, nitro, sulfenylamino, sulfinylamino, sulfonylamino, aryl, aryl-C₁-C₄-alkyl, heteroaryl, heteroaryl-C₁-C₄-alkyl, heterocyclyl, heterocyclyl-C₁-C₄-alkyl, C₁-C₆-alkyl, C₃-C₈-cycloalkyl, C₁-C₆-alkoxy, C₁-C₆-alkoxycarbonyl, C₁-C₆-alkylcarbonyl, (C₁-C₆-alkyl)amino, di(C₁-C₆-alkyl)amino, C₁-C₆-alkylthio, C₁-C₆-alkylsulfinyl and C₁-C₆-alkylsulfonyl, wherein the ten last-mentioned radicals may be unsubstituted, partially or fully halogenated and/or may carry 1, 2 or 3 radicals, selected from the group consisting of cyano, nitro, amino, OH, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, C₁-C₄-alkylcarbonyloxy, (C₁-C₄-alkoxy)carbonyl, (C₁-C₄-alkyl)amino, di(C₁-C₄-alkyl)amino, C₃-C₈-cycloalkyl and phenyl, it being possible for phenyl to be unsubstituted, partially or fully halogenated and/or to carry 1, 2 or 3 substituents, independently of one another selected from the group consisting of C₁-C₄-alkyl, C₁-C₄-haloalkyl, C₁-C₄-alkoxy and C₁-C₄-haloalkoxy,
wherein the heteroaryl moiety in heteroaryl and heteroaryl-C₁-C₄-alkyl of R¹³ is 5- or 6 membered and contains 1, 2, 3 or 4 heteroatoms and/or heteroatom groups, selected from the group consisting of O, S, SO, SO₂, N and NR^{g}, as ring members, R^{g} being hydrogen or C₁-C₆-alkyl,
wherein the heterocyclyl moiety in heterocyclyl and heterocyclyl-C₁-C₄-alkyl of R¹³ is 3- to 7-membered and contains 1, 2, 3 or 4 heteroatoms and/or heteroatom groups, selected from O, S, SO, SO₂, N or NR^{h}, as ring members, R^{h} being hydrogen or C₁-C₆-alkyl, and additionally may contain 1, 2 or 3 CO groups as ring members,
and wherein the carbon atoms of aryl, hetaryl, and heterocyclyl in R¹³ may be unsubstituted or may carry 1, 2 or 3 substituents, independently of one another selected from the group consisting of halogen, C₁-C₄-alkyl, C₁-C₄-haloalkyl, C₁-C₄-alkoxy and C₁-C₄-haloalkoxy;
R¹⁴ and R¹⁵, independently of one another, are selected from the group consisting of hydrogen, hydroxy, C₁-C₆-alkoxy, amino, C₁-C₄-alkylamino, di(C₁-C₄-alkyl)amino, aryl, aryl-C₁-C₄-alkyl, and C₁-C₄-alkyl, where the alkyl moiety in the two last-mentioned radicals and the aryl moiety in aryl or aryl-C₁-C₄-alkyl may be partially or fully halogenated,
3- to 7-membered heteroaryl or heteroaryl-C₁-C₄-alkyl, wherein the heteroaryl ring contains as ring members 1, 2 or 3 heteroatoms and/or heteroatom groups, selected from the group consisting of nitrogen, oxygen, sulfur, SO, SO₂ and N—Rⁿ, wherein Rⁿ is hydrogen or C₁-C₄-alkyl,
3- to 7-membered heterocyclyl or heterocyclyl-C₁-C₄-alkyl, wherein the heterocyclic ring is saturated or partly unsaturated and contains 1, 2 or 3 heteroatoms and/or heteroatom groups, selected from the group consisting of nitrogen, oxygen, sulfur, group SO, SO₂ and N—R^{o}, wherein R^{o} is hydrogen or C₁-C₄-alkyl,
and wherein the carbon atoms of the heterocyclic rings may be unsubstituted or substituted by 1 or 2 radicals are halogen or C₁-C₄-alkyl;
R¹⁶ is C₁-C₆-alkyl, aryl, aryl-C₁-C₄-alkyl,
5- to 7-membered heteroaryl or heteroaryl-C₁-C₄-alkyl, wherein the heteroaryl ring contains as ring members 1, 2 or 3 heteroatoms and/or heteroatom groups, selected from the group consisting of nitrogen, oxygen, sulfur, SO, SO₂ and N—R^{k}, wherein R^{k} is hydrogen or C₁-C₄-alkyl,
3- to 7-membered heterocyclyl or heterocyclyl-C₁-C₄-alkyl, wherein the heterocyclic ring is saturated or partly unsaturated and contains as ring members 1, 2 or 3 heteroatoms and/or heteroatom groups, selected from the group consisting of nitrogen, oxygen, sulfur, SO, SO₂ and N—R^{m}, wherein R^{m} is hydrogen or C₁-C₄-alkyl,
and wherein the carbon atoms of the heterocyclic rings may be unsubstituted or substituted by 1 or 2 radicals selected from halogen or C₁-C₄-alkyl;
or
R¹⁶ and R⁹ together with the adjacent nitrogen and carbon atoms form a saturated or ethylenically unsaturated 5 to 10-membered ring, where the ring may be partially or fully halogenated and/or may be substituted by 1, 2 or 3 substituents selected from the group consisting of C₁-C₅-alkyl and C₁-C₅-haloalkyl, wherein the ring may contain, in addition to the nitrogen and carbon ring members, 1, 2 or 3 heteroatoms and/or heteroatom groups as ring members selected from the group consisting of nitrogen, oxygen, sulfur, SO, SO₂ and N—R¹⁷;
each R¹⁷ independently is hydrogen, heteroaryl, heteroaryl-C₁-C₄-alkyl or C₁-C₆-alkyl, where the alkyl moiety in the two last-mentioned radicals may be partially or fully halogenated;
and/or the agriculturally acceptable salts thereof.

30. The method of claim 29, wherein m is 2.

31. The method of claim 29, wherein R¹ is different from hydrogen.

32. The method of claim 29, wherein R¹ is selected from the group consisting of halogen, C₁-C₄-alkyl, C₁-C₄-haloalkyl, C₁-C₄-alkoxy and C₁-C₄-haloalkoxy.

33. The method of claim 29, wherein two of the radicals R², R³ and R⁴ are hydrogen and the remaining radical of R², R³ or R⁴ is selected from the group consisting of halogen, C₁-C₄-alkyl, and C₁-C₄-haloalkyl.

34. The method of claim 29, wherein all of the radicals R², R³ and R⁴ are hydrogen.

35. The method of claim 29, wherein A is a radical N═CR⁵R⁶.

36. The method of claim 35, wherein
R⁵ is C₁-C₆-alkyl or a radical OR^{5a}, and
R⁶ is a radical OR^{6a}
wherein R^{5a} and R^{6a} have, independently of each other, one of the meanings given above for R^{5a}.

37. The method of claim 36, wherein R^{5a} and R^{6a} are, independently of each other, selected from the group consisting of C₁-C₆-alkyl, C₂-C₆-alkenyl, C₂-C₆-alkynyl, C₁-C₄-alkoxy-C₁-C₄-alkyl and C₁-C₄-haloalkyl.

38. The method of claim 35, wherein
R⁵ is C₁-C₆-alkyl, which may be unsubstituted, partially or fully halogenated and/or may carry 1, 2 or 3 radicals, selected from the group consisting of cyano, nitro, amino, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, (C₁-C₄-alkoxy)carbonyl, (C₁-C₄-alkyl)amino, di(C₁-C₄-alkyl)amino and C₃-C₈-cycloalkyl, or is phenyl or phenyl-C₁-C₄-alkyl, wherein the phenyl group in the two last-mentioned radicals may be unsubstituted or may carry 1, 2 or 3 substituents, independently of one another selected from the group consisting of halogen, C₁-C₄-alkyl, C₁-C₄-haloalkyl, C₁-C₄-alkoxy and C₁-C₄-haloalkoxy; and
R⁶ is hydrogen, C₁-C₆-alkyl, which may be unsubstituted, partially or fully halogenated and/or may carry 1, 2 or 3 radicals, selected from the group consisting of cyano, nitro, amino, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, (C₁-C₄-alkoxy)carbonyl, (C₁-C₄-alkyl)amino, di(C₁-C₄-alkyl)amino and C₃-C₈-cycloalkyl, or is phenyl or phenyl-C₁-C₄-alkyl, wherein the phenyl group in the two last-mentioned radicals may be unsubstituted or may carry 1, 2 or 3 substituents, independently of one another selected from the group consisting of halogen, C₁-C₄-alkyl, C₁-C₄-haloalkyl, C₁-C₄-alkoxy and C₁-C₄-haloalkoxy.

39. The method of claim 38, wherein
R⁵ is selected from the group consisting of C₁-C₆-alkyl, C₁-C₄-alkoxy-C₁-C₄-alkyl, C₁-C₄-haloalkyl and phenyl-C₁-C₄-alkyl, wherein phenyl may be unsubstituted, partially or fully halogenated and/or carry 1, 2 or 3 substituents, independently of one another selected from the group consisting of C₁-C₄-alkyl, C₁-C₄-haloalkyl, C₁-C₄-alkoxy and C₁-C₄-haloalkoxy; and
R⁶ is selected from the group consisting of hydrogen, C₁-C₆-alkyl, C₁-C₄-alkoxy-C₁-C₄-alkyl, C₁-C₄-haloalkyl and phenyl-C₁-C₄-alkyl, wherein phenyl may be unsubstituted, partially or fully halogenated and/or carry 1, 2 or 3 substituents, independently of one another selected from the group consisting of C₁-C₄-alkyl, C₁-C₄-haloalkyl, C₁-C₄-alkoxy and C₁-C₄-haloalkoxy.

40. The method of claim 35, wherein
R⁵ is a radical NR^{5b}R^{5c}, and
R⁶ is hydrogen, C₁-C₆-alkyl, which may be unsubstituted, partially or fully halogenated and/or may carry 1, 2 or 3 radicals, selected from the group consisting of cyano, nitro, amino, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, (C₁-C₄-alkoxy)carbonyl, (C₁-C₄-alkyl)amino, di(C₁-C₄-alkyl)amino and C₃-C₈-cycloalkyl, or is phenyl or phenyl-C₁-C₄-alkyl, wherein the phenyl group in the two last-mentioned radicals may be unsubstituted or may carry 1, 2 or 3 substituents, independently of one another selected from the group consisting of halogen, C₁-C₄-alkyl, C₁-C₄-haloalkyl, C₁-C₄-alkoxy and C₁-C₄-haloalkoxy, or a radical OR^{6a},
wherein R^{6a} has one of the meanings given above for R^{5a} and
R^{5b} and R^{5c} are as defined above.

41. The method of claim 29, wherein A is a radical N═SR⁷R⁸.

42. The method of claim 41, wherein R⁷ and R⁸ are, independently of each other, selected from C₁-C₆-alkyl, C₂-C₆-alkenyl, C₂-C₆-alkynyl, C₁-C₄-alkoxy-C₁-C₄-alkyl, C₁-C₄-haloalkyl, phenyl and phenyl-C₁-C₄-alkyl, wherein phenyl in the last two radicals may be unsubstituted, partially or fully halogenated and/or carry 1, 2 or 3 substituents, independently of one another selected from the group consisting of C₁-C₄-alkyl, C₁-C₄-haloalkyl, C₁-C₄-alkoxy and C₁-C₄-haloalkoxy,
or R⁷ and R⁸ together form a moiety (CH₂)ₖ, wherein k is 4, 5 or 6 and wherein 1, 2, 3 or 4 hydrogen atoms may be replaced by C₁-C₄-alkyl or halogen and wherein 1 or 2 non-adjacent CH₂ moieties may be replaced by a carbonyl group, a heteroatom or a heteroatom group, selected from O, S, SO₂ and N—R^{#} with R^{#} being H or C₁-C₄-alkyl.

43. The method of claim 29, wherein A is a radical of the formula NR¹⁰—C(═O)—R⁹ wherein
R¹⁰ is selected from the group consisting of hydrogen, C₁-C₆-alkyl, C₂-C₆-alkenyl, C₂-C₆-alkynyl, C₁-C₆-alkoxy, C₁-C₄-alkoxy-C₁-C₄-alkyl, C₁-C₄-haloalkyl and acetyl; and
R⁹ is selected from the group consisting of C₁-C₆-alkyl, C₂-C₆-alkenyl, C₂-C₆-alkynyl, C₁-C₄-alkoxy-C₁-C₄-alkyl, C₁-C₄-haloalkyl, phenyl and phenyl-C₁-C₄-alkyl, wherein phenyl in the last to radicals may be unsubstituted, partially or fully halogenated and/or carry 1, 2 or 3 substituents, independently of one another selected from the group consisting of C₁-C₄-alkyl, C₁-C₄-haloalkyl, C₁-C₄-alkoxy and C₁-C₄-haloalkoxy, or
R¹⁰ and R⁹ together form a moiety of the (CH₂)ₚ, wherein p is 3, 4 or 5 and wherein 1, 2, 3 or 4 hydrogen atoms may be replaced by C₁-C₄-alkyl or halogen and wherein 1 or 2 non-adjacent CH₂ moieties may be replaced by a carbonyl group, a heteroatom or a heteroatom group, selected from O, S, SO₂ and N—R^{#} with R^{#} being H or C₁-C₄-alkyl.

44. The method of claim 43, wherein
R¹⁰ is selected from the group consisting of hydrogen, C₁-C₄-alkyl, C₂-C₆-alkynyl and C₁-C₄-alkoxy; and
R⁹ is selected from the group consisting of C₁-C₄-alkyl, C₂-C₆-alkenyl, C₁-C₄-haloalkyl and phenyl, or
R¹⁰ and R⁹ together form a moiety of the (CH₂)ₚ, wherein p is 3 or 4.

45. The method of claim 43, wherein
R⁹ is C₁-C₆-alkyl or C₁-C₄-haloalkyl, and
R¹⁰ is hydrogen or C₁-C₆-alkyl.

46. The method of claim 29, wherein A is a radical of the formula NR¹⁰—C(═O)—R⁹ wherein
R¹⁰ is selected from the group consisting of hydrogen, C₁-C₆-alkyl, C₂-C₆-alkenyl, C₂-C₆-alkynyl, C₁-C₆-alkoxy, C₁-C₄-alkoxy-C₁-C₄-alkyl and C₁-C₄-haloalkyl; and
R⁹ is OR^{9a} or a radical NR^{9b}R^{9c}.

47. The method of claim 29, wherein A is a N-bound 5-membered aromatic heterocycle, which additionally may contain 1, 2, or 3 nitrogen atoms as ring members and which may carry 1, 2, 3 or 4 radicals R¹³.

48. The method of claim 47, wherein A is 1-pyrrolyl, 1-pyrazolyl, 1-imidazolyl or [1,2,4]-triazol-1-yl, where the heterocycle may be unsubstituted or may carry 1, 2 or 3 substituents selected from the group consisting of halogen and C₁-C₄-alkyl, wherein C₁-C₄-alkyl may be unsubstituted or may be substituted by hydroxy or acetyloxy.

49. A method of protecting crops from attack or infestation by animal pests comprising,
contacting a crop with a pesticidally effective amount of at least one compound of the formula I and/or at least one salt thereof: 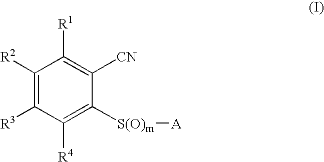
wherein
m is 0, 1 or 2;
A is a radical of the formulae
N═CR⁵R⁶, N═SR⁷R⁸, NR¹⁰—C(═X)—R⁹,
where X is O or S,
or A is a N-bound 5-, 6- or 7-membered heterocycle, which is ethylenically unsaturated or aromatic, and which additionally may contain 1, 2, or 3 further heteroatoms or heteroatom groups, selected from the group consisting of O, S, SO, SO₂, N, and NR¹², and/or 1, 2 or 3 carbonyl groups as ring members and which may carry 1, 2, 3 or 4 radicals R¹³,
R¹ is hydrogen, nitro, cyano, azido, amino, halogen, sulfenylamino, sulfinylamino, sulfonylamino, C(═O)R¹⁴, C₄-C₆-alkyl, C₂-C₆-alkenyl, C₂-C₆-alkynyl, C₃-C₈-cycloalkyl, C₁-C₆-alkoxy, (C₁-C₆-alkyl)amino, di(C₁-C₆-alkyl)amino, C₁-C₆-alkylthio, C₁-C₆-alkylsulfinyl or C₁-C₆-alkylsulfonyl, wherein the ten last-mentioned radicals may be unsubstituted, partially or fully halogenated and/or may carry 1, 2 or 3 radicals, selected from the group consisting of cyano, nitro, amino, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, (C₁-C₄-alkoxy)carbonyl, (C₁-C₄-alkyl)amino, di(C₁-C₄-alkyl)amino, C₃-C₈-cycloalkyl and phenyl, it being possible for phenyl to be unsubstituted, partially or fully halogenated and/or to carry 1, 2 or 3 substituents, independently of one another selected from the group consisting of C₁-C₄-alkyl, C₁-C₄-haloalkyl, C₁-C₄-alkoxy and C₁-C₄-haloalkoxy;
R², R³ and R⁴ are independently of one another selected from the group consisting of hydrogen, halogen, cyano, azido, nitro, C₁-C₆-alkyl, C₃-C₈-cycloalkyl, C₁-C₄-haloalkyl, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, C₂-C₆-alkenyl, C₂-C₆-alkynyl, amino, (C₁-C₄-alkyl)amino, di(C₁-C₄-alkyl)amino, sulfonylamino, sulfinylamino, sulfenylamino and C(═O)—R¹⁵;
R⁵ is H, OR^{5a}, NR^{5b}R^{5c}, aryl, aryl-C₁-C₄-alkyl, heteroaryl, heteroaryl-C₁-C₄-alkyl, heterocyclyl, heterocyclyl-C₁-C₄-alkyl, C₁-C₁₀-alkyl, C₃-C₁₀-cycloalkyl, C₁-C₁₀-alkylthio, C₁-C₁₀-alkylsulfonyl, or C₁-C₁₀-alkylsulfinyl, wherein the five last-mentioned radicals may be unsubstituted, partially or fully halogenated and/or may carry 1, 2 or 3 radicals, independently of one another each selected from the group consisting of cyano, nitro, amino, C₁-C₁₀-alkoxy, C₁-C₁₀-alkylthio, C₁-C₁₀-alkylsulfinyl, C₁-C₁₀-alkylsulfonyl, C₁-C₁₀-haloalkoxy, C₁-C₁₀-haloalkylthio, C₁-C₁₀-alkoxycarbonyl, (C₁-C₁₀-alkyl)amino, di-(C₁-C₁₀-alkyl)amino, C₃-C₁₀-cycloalkyl and phenyl, it being possible for phenyl to be unsubstituted, partially or fully halogenated and/or to carry 1, 2 or 3 substituents, independently of one another selected from the group consisting of C₁-C₄-alkyl, C₁-C₄-haloalkyl, C₁-C₄-alkoxy and C₁-C₄-haloalkoxy;
wherein
R^{5a} is C₁-C₁₀-alkyl, C₃-C₁₀-cycloalkyl, C₂-C₁₀-alkenyl, C₂-C₁₀-alkynyl, aryl, aryl-C₁-C₄-alkyl, heteroaryl or heteroaryl-C₁-C₄-alkyl, heterocyclyl or heterocyclyl-C₁-C₄-alkyl;
R^{5b}, R^{5c}, independently from each other, are hydrogen, C₁-C₁₀-alkyl, C₃-C₁₀-cycloalkyl, C₂-C₁₀-alkenyl, C₂-C₁₀-alkynyl, aryl, aryl-C₁-C₄-alkyl, heteroaryl and heteroaryl-C₁-C₄-alkyl, heterocyclyl or heterocyclyl-C₁-C₄-alkyl;
wherein C₁-C₁₀-alkyl, C₃-C₁₀-cycloalkyl, C₂-C₁₀-alkenyl and C₂-C₁₀-alkynyl in R^{5a}, R^{5b} and R^{5c} may be unsubstituted, partially or fully halogenated and/or may carry 1, 2 or 3 radicals, selected from the group consisting of cyano, nitro, amino, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, (C₁-C₄-alkoxy)carbonyl, (C₁-C₄-alkyl)amino, di(C₁-C₄-alkyl)amino and C₃-C₈-cycloalkyl;
wherein the heteroaryl moiety in heteroaryl and heteroaryl-C₁-C₄-alkyl of R⁵, R^{5a}, R^{5b} and R^{5c} is 5- or 6 membered and contains 1, 2, 3 or 4 heteroatoms and/or heteroatom groups, selected from the group consisting of O, S, SO, SO₂, N and NR^{a}, as ring members, R^{a} being hydrogen or C₁-C₆-alkyl;
wherein the heterocyclyl moiety in heterocyclyl and heterocyclyl-C₁-C₄-alkyl of R⁵, R^{5a}, R^{5b} and R^{5c} is 3- to 7 membered, is saturated or partly unsaturated and contains 1, 2, 3 or 4 heteroatoms and/or heteroatom groups, selected from the group consisting of O, S, SO, SO₂, N and NR^{b}, as ring members, R^{b} being hydrogen or C₁-C₆-alkyl, and additionally may contain 1, 2 or 3 CO groups as ring members;
and wherein the carbon atoms of aryl, hetaryl, and heterocyclyl moieties in R⁵, R^{5a}, R^{5b} and R^{5c} may be unsubstituted or may carry 1, 2 or 3 substituents, independently of one another selected from the group consisting of halogen, C₁-C₄-alkyl, C₁-C₄-haloalkyl, C₁-C₄-alkoxy and C₁-C₄-haloalkoxy;
R⁶ independently has one of the meanings given for R⁵;
R⁷, R⁸, independently from each other, are selected from the group consisting of aryl, aryl-C₁-C₄-alkyl, heteroaryl, heteroaryl-C₁-C₄-alkyl, heterocyclyl, heterocyclyl-C₁-C₄-alkyl, C₁-C₁₀-alkyl, C₃-C₁₀-cycloalkyl, C₂-C₁₀-alkenyl and C₂-C₁₀-alkynyl, wherein the four last-mentioned radicals may be unsubstituted, partially or fully halogenated and/or may carry 1, 2 or 3 radicals, independently of one another each selected from the group consisting of cyano, nitro, amino, C₁-C₁₀-alkoxy, C₁-C₁₀-alkylthio, C₁-C₁₀-alkylsulfinyl, C₁-C₁₀-alkylsulfonyl, C₁-C₁₀-haloalkoxy, C₁-C₁₀-haloalkylthio, C₁-C₁₀-alkoxycarbonyl, (C₁-C₁₀-alkyl)amino, di-(C₁-C₁₀-alkyl)amino, C₃-C₁₀-cycloalkyl and phenyl, it being possible for phenyl to be unsubstituted, partially or fully halogenated and/or to carry 1, 2 or 3 substituents, independently of one another selected from the group consisting of C₁-C₄-alkyl, C₁-C₄-haloalkyl, C₁-C₄-alkoxy and C₁-C₄-haloalkoxy; or
R⁷ and R⁸ together with the sulfur atom they are bound to form a saturated or ethylenically unsaturated 5- to 10-membered ring, optionally substituted by 1, 2, 3 or 4 radicals selected from the group consisting of C₁-C₅-alkyl and halogen, wherein the ring may contain, in addition to the sulfur atom, 1, 2 or 3 heteroatoms and/or heteroatom-containing groups as ring members selected from the group consisting of nitrogen, oxygen, sulfur, CO, SO, SO₂ and N—R¹⁷;
wherein the heteroaryl moiety in heteroaryl and heteroaryl-C₁-C₄-alkyl of R⁷ and R⁸ is 5- or 6 membered and contains 1, 2, 3 or 4 heteroatoms and/or heteroatom groups, selected from the group consisting of O, S, SO, SO₂, N and NR^{c}, as ring members, R^{c} being hydrogen or C₁-C₆-alkyl,
wherein the heterocyclyl moiety in heterocyclyl and heterocyclyl-C₁-C₄-alkyl of R⁷ and R⁸ is 3- to 7-membered, is saturated or partly unsaturated and contains 1, 2, 3 or 4 heteroatoms and/or heteroatom groups, selected from the group consisting of O, S, SO, SO₂, N and NR^{d}, as ring members, R^{d} being hydrogen or C₁-C₆-alkyl, and additionally may contain 1, 2 or 3 CO groups as ring members,
and wherein the carbon atoms of aryl, hetaryl, and heterocyclyl in R⁷ and R⁸ may be unsubstituted or may carry 1, 2 or 3 substituents, independently of one another selected from the group consisting of halogen, C₁-C₄-alkyl, C₁-C₄-haloalkyl, C₁-C₄-alkoxy and C₁-C₄-haloalkoxy;
R⁹ is selected from the group consisting of hydrogen, OR^{9a}, NR^{9b}R^{9c}, aryl, aryl-C₁-C₄-alkyl, heteroaryl, heteroaryl-C₁-C₄-alkyl, heterocyclyl, heterocyclyl-C₁-C₄-alkyl, C₁-C₁₀-alkyl, C₂-C₁₀-alkenyl, C₂-C₁₀-alkynyl and C₃-C₁₀-cycloalkyl, wherein the four last-mentioned radicals may be unsubstituted, partially or fully halogenated and/or may carry 1, 2 or 3 radicals, independently of one another each selected from the group consisting of cyano, nitro, amino, C₁-C₁₀-alkoxy, C₁-C₁₀-alkylthio, C₁-C₁₀-alkylsulfinyl, C₁-C₁₀-alkylsulfonyl, C₁-C₁₀-haloalkoxy, C₁-C₁₀-haloalkylthio, C₁-C₁₀-alkoxycarbonyl, (C₁-C₁₀-alkyl)amino, di-(C₁-C₁₀ alkyl)amino, C₃-C₁₀-cycloalkyl and phenyl, it being possible for phenyl to be unsubstituted, partially or fully halogenated and/or to carry 1, 2 or 3 substituents, independently of one another selected from the group consisting of C₁-C₄-alkyl, C₁-C₄-haloalkyl, C₁-C₄-alkoxy and C₁-C₄-haloalkoxy,
and wherein
R^{9a} is C₁-C₁₀-alkyl, C₃-C₁₀-cycloalkyl, C₂-C₁₀-alkenyl, C₂-C₁₀-alkynyl, aryl, aryl-C₁-C₄-alkyl, heteroaryl or heteroaryl-C₁-C₄-alkyl, heterocyclyl or heterocyclyl-C₁-C₄-alkyl,
R^{9b}, R^{9c}, independently from each other, are selected from the group consisting of hydrogen, C₁-C₁₀-alkyl, C₃-C₁₀-cycloalkyl, C₂-C₁₀-alkenyl, C₂-C₁₀-alkynyl, aryl, aryl-C₁-C₄-alkyl, heteroaryl and heteroaryl-C₁-C₄-alkyl, heterocyclyl and heterocyclyl-C₁-C₄-alkyl,
wherein C₁-C₁₀-alkyl, C₃-C₁₀-cycloalkyl, C₂-C₁₀-alkenyl and C₂-C₁₀-alkynyl in R^{9a}, R^{9b} and R^{9c} may be unsubstituted, partially or fully halogenated and/or may carry 1, 2 or 3 radicals, selected from the group consisting of cyano, nitro, amino, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, (C₁-C₄-alkoxy)carbonyl, (C₁-C₄-alkyl)amino, di(C₁-C₄-alkyl)amino and C₃-C₈-cycloalkyl,
wherein the heteroaryl moiety in heteroaryl and heteroaryl-C₁-C₄-alkyl of R⁹, R^{9a}, R^{9b} and R^{9c} is 5- or 6-membered and contains 1, 2, 3 or 4 heteroatoms and/or heteroatom groups, selected from the group consisting of O, S, SO, SO₂, N and NR^{e}, as ring members, R^{e} being hydrogen or C₁-C₆-alkyl,
wherein the heterocyclyl moiety in heterocyclyl and heterocyclyl-C₁-C₄-alkyl of R⁹, R^{9a}, R^{9b} and R^{9c} is 3- to 7-membered, is saturated or partly unsaturated and contains 1, 2, 3 or 4 heteroatoms and/or heteroatom groups, selected from the group consisting of O, S, SO, SO₂, N and NR^{f}, as ring members, R^{f} being hydrogen or C₁-C₆-alkyl, and additionally may contain 1, 2 or 3 CO groups as ring members,
and wherein the carbon atoms of aryl, hetaryl and heterocyclyl moieties in R⁹, R^{9a}, R^{9b} and R^{9c} may be unsubstituted or may carry 1, 2 or 3 substituents, independently of one another selected from the group consisting of halogen, C₁-C₄-alkyl, C₁-C₄-haloalkyl, C₁-C₄-alkoxy and C₁-C₄-haloalkoxy;
R¹⁰ is selected from the group consisting of hydrogen, C(═O)—R¹⁶, C₁-C₁₀-alkyl, C₂-C₁₀-alkenyl, C₂-C₁₀-alkynyl, C₁-C₁₀-alkoxy and C₃-C₁₀-cycloalkyl, wherein the five last-mentioned radicals may be unsubstituted, partially or fully halogenated and/or may carry 1, 2 or 3 radicals, independently of one another each selected from the group consisting of cyano, nitro, amino, C₁-C₁₀-alkoxy, C₁-C₁₀-alkylthio, C₁-C₁₀-alkylsulfinyl, C₁-C₁₀-alkylsulfonyl, C₁-C₁₀-haloalkoxy, C₁-C₁₀-haloalkylthio, C₁-C₁₀-alkoxycarbonyl, (C₁-C₁₀-alkyl)amino, di-(C₁-C₁₀-alkyl)amino, C₃-C₁₀-cycloalkyl and phenyl, it being possible for phenyl to be unsubstituted, partially or fully halogenated and/or to carry 1, 2 or 3 substituents, independently of one another selected from the group consisting of C₁-C₄-alkyl, C₁-C₄-haloalkyl, C₁-C₄-alkoxy and C₁-C₄-haloalkoxy; or
R⁹ and R¹⁰ together with the adjacent nitrogen and carbon atoms form a saturated or ethylenically unsaturated 5 to 10-membered ring, optionally substituted by 1, 2, 3 or 4 radicals selected from C₁-C₅-alkyl and halogen, wherein the ring may contain, in addition to the nitrogen and carbon ring members, 1, 2 or 3 heteroatoms and/or heteroatom groups as ring members selected from the group consisting of nitrogen, oxygen, sulfur, CO, SO, SO₂ and N—R¹⁷;
R¹² is hydrogen, C₁-C₆-alkyl, C₁-C₄-alkoxy-C₁-C₄-alkyl or C₁-C₆-alkoxy;
each R¹³ independently is selected from the group consisting of halogen, cyano, nitro, sulfenylamino, sulfinylamino, sulfonylamino, aryl, aryl-C₁-C₄-alkyl, heteroaryl, heteroaryl-C₁-C₄-alkyl, heterocyclyl, heterocyclyl-C₁-C₄-alkyl, C₁-C₆-alkyl, C₃-C₈-cycloalkyl, C₁-C₆-alkoxy, C₁-C₆-alkoxycarbonyl, C₁-C₆-alkylcarbonyl, (C₁-C₆-alkyl)amino, di(C₁-C₆-alkyl)amino, C₁-C₆-alkylthio, C₁-C₆-alkylsulfinyl and C₁-C₆-alkylsulfonyl, wherein the ten last-mentioned radicals may be unsubstituted, partially or fully halogenated and/or may carry 1, 2 or 3 radicals, selected from the group consisting of cyano, nitro, amino, OH, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, C₁-C₄-alkylcarbonyloxy, (C₁-C₄-alkoxy)carbonyl, (C₁-C₄-alkyl)amino, di(C₁-C₄-alkyl)amino, C₃-C₈-cycloalkyl and phenyl, it being possible for phenyl to be unsubstituted, partially or fully halogenated and/or to carry 1, 2 or 3 substituents, independently of one another selected from the group consisting of C₁-C₄-alkyl, C₁-C₄-haloalkyl, C₁-C₄-alkoxy and C₁-C₄-haloalkoxy,
wherein the heteroaryl moiety in heteroaryl and heteroaryl-C₁-C₄-alkyl of R¹³ is 5- or 6 membered and contains 1, 2, 3 or 4 heteroatoms and/or heteroatom groups, selected from the group consisting of O, S, SO, SO₂, N and NR^{g}, as ring members, R^{g} being hydrogen or C₁-C₆-alkyl,
wherein the heterocyclyl moiety in heterocyclyl and heterocyclyl-C₁-C₄-alkyl of R¹³ is 3- to 7-membered and contains 1, 2, 3 or 4 heteroatoms and/or heteroatom groups, selected from O, S, SO, SO₂, N or NR^{h}, as ring members, R^{h} being hydrogen or C₁-C₆-alkyl, and additionally may contain 1, 2 or 3 CO groups as ring members,
and wherein the carbon atoms of aryl, hetaryl, and heterocyclyl in R¹³ may be unsubstituted or may carry 1, 2 or 3 substituents, independently of one another selected from the group consisting of halogen, C₁-C₄-alkyl, C₁-C₄-haloalkyl, C₁-C₄-alkoxy and C₁-C₄-haloalkoxy;
R¹⁴ and R⁵, independently of one another, are selected from the group consisting of hydrogen, hydroxy, C₁-C₆-alkoxy, amino, C₁-C₄-alkylamino, di(C₁-C₄-alkyl)amino, aryl, aryl-C₁-C₄-alkyl, and C₁-C₄-alkyl, where the alkyl moiety in the two last-mentioned radicals and the aryl moiety in aryl or aryl-C₁-C₄-alkyl may be partially or fully halogenated,
3- to 7-membered heteroaryl or heteroaryl-C₁-C₄-alkyl, wherein the heteroaryl ring contains as ring members 1, 2 or 3 heteroatoms and/or heteroatom groups, selected from the group consisting of nitrogen, oxygen, sulfur, SO, SO₂ and N—Rⁿ, wherein Rⁿ is hydrogen or C₁-C₄-alkyl,
3- to 7-membered heterocyclyl or heterocyclyl-C₁-C₄-alkyl, wherein the heterocyclic ring is saturated or partly unsaturated and contains 1, 2 or 3 heteroatoms and/or heteroatom groups, selected from the group consisting of nitrogen, oxygen, sulfur, group SO, SO₂ and N—R^{o}, wherein R^{o} is hydrogen or C₁-C₄-alkyl,
and wherein the carbon atoms of the heterocyclic rings may be unsubstituted or substituted by 1 or 2 radicals selected from halogen or C₁-C₄-alkyl;
R¹⁶ is C₁-C₆-alkyl, aryl, aryl-C₁-C₄-alkyl,
5- to 7-membered heteroaryl or heteroaryl-C₁-C₄-alkyl, wherein the heteroaryl ring contains as ring members 1, 2 or 3 heteroatoms and/or heteroatom groups, selected from the group consisting of nitrogen, oxygen, sulfur, SO, SO₂ and N—R^{k}, wherein R^{k} is hydrogen or C₁-C₄-alkyl,
3- to 7-membered heterocyclyl or heterocyclyl-C₁-C₄-alkyl, wherein the heterocyclic ring is saturated or partly unsaturated and contains as ring members 1, 2 or 3 heteroatoms and/or heteroatom groups, selected from the group consisting of nitrogen, oxygen, sulfur, SO, SO₂ and N—R^{m}, wherein R^{m} is hydrogen or C₁-C₄-alkyl,
and wherein the carbon atoms of the heterocyclic rings may be unsubstituted or substituted by 1 or 2 radicals selected from the group consisting of halogen and C₁-C₄-alkyl;
or
R¹⁶ and R⁹ together with the adjacent nitrogen and carbon atoms form a saturated or ethylenically unsaturated 5 to 10-membered ring, where the ring may be partially or fully halogenated and/or may be substituted by 1, 2 or 3 substituents selected from the group consisting of C₁-C₅-alkyl and C₁-C₅-haloalkyl, wherein the ring may contain, in addition to the nitrogen and carbon ring members, 1, 2 or 3 heteroatoms and/or heteroatom groups as ring members selected from the group consisting of nitrogen, oxygen, sulfur, SO, SO₂ and N—R¹⁷;
each R¹⁷ independently is hydrogen, heteroaryl, heteroaryl-C₁-C₄-alkyl or C₁-C₆-alkyl, where the alkyl moiety in the two last-mentioned radicals may be partially or fully halogenated;
and/or the agriculturally acceptable salts thereof.

50. A method for protecting seeds from soil insects and of the seedlings' roots and shoots from insects comprising,
contacting the seeds before sowing and/or after pregermination with a compound of the formula I and/or at least one agriculturally acceptable salt thereof in pesticidally effective amounts: 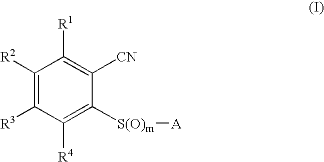
wherein
m is 0, 1 or 2;
A is a radical of the formulae
N═CR⁵R⁶, N═SR⁷R⁸, NR¹⁰—C(═X)—R⁹,
where X is O or S,
or A is a N-bound 5-, 6- or 7-membered heterocycle, which is ethylenically unsaturated or aromatic, and which additionally may contain 1, 2, or 3 further heteroatoms or heteroatom groups, selected from O, S, SO, SO₂, N, and NR¹² and/or 1, 2 or 3 carbonyl groups as ring members and which may carry 1, 2, 3 or 4 radicals R¹³,
R¹ is hydrogen, nitro, cyano, azido, amino, halogen, sulfenylamino, sulfinylamino, sulfonylamino, C(═O)R¹⁴, C₁-C₆-alkyl, C₂-C₆-alkenyl, C₂-C₆-alkynyl, C₃-C₈-cycloalkyl, C₁-C₆-alkoxy, (C₁-C₆-alkyl)amino, di(C₁-C₆-alkyl)amino, C₁-C₆-alkylthio, C₁-C₆-alkylsulfinyl or C₁-C₆-alkylsulfonyl, wherein the ten last-mentioned radicals may be unsubstituted, partially or fully halogenated and/or may carry 1, 2 or 3 radicals, selected from the group consisting of cyano, nitro, amino, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, (C₁-C₄-alkoxy)carbonyl, (C₁-C₄-alkyl)amino, di(C₁-C₄-alkyl)amino, C₃-C₈-cycloalkyl and phenyl, it being possible for phenyl to be unsubstituted, partially or fully halogenated and/or to carry 1, 2 or 3 substituents, independently of one another selected from the group consisting of C₁-C₄-alkyl, C₁-C₄-haloalkyl, C₁-C₄-alkoxy and C₁-C₄-haloalkoxy;
R², R³ and R⁴ are independently of one another selected from the group consisting of hydrogen, halogen, cyano, azido, nitro, C₁-C₆-alkyl, C₃-C₈-cycloalkyl, C₁-C₄-haloalkyl, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, C₂-C₆-alkenyl, C₂-C₆-alkynyl, amino, (C₁-C₄-alkyl)amino, di(C₁-C₄-alkyl)amino, sulfonylamino, sulfinylamino, sulfenylamino and C(═O)—R¹⁵;
R⁵ is H, OR^{5a}, NR^{5b}R^{5c}, aryl, aryl-C₁-C₄-alkyl, heteroaryl, heteroaryl-C₁-C₄-alkyl, heterocyclyl, heterocyclyl-C₁-C₄-alkyl, C₁-C₁₀-alkyl, C₃-C₁₀-cycloalkyl, C₁-C₁₀-alkylthio, C₁-C₁₀-alkylsulfonyl, C₁-C₁₀-alkylsulfinyl, wherein the five last-mentioned radicals may be unsubstituted, partially or fully halogenated and/or may carry 1, 2 or 3 radicals, independently of one another each selected from the group consisting of cyano, nitro, amino, C₁-C₁₀-alkoxy, C₁-C₁₀-alkylthio, C₁-C₁₀-alkylsulfinyl, C₁-C₁₀-alkylsulfonyl, C₁-C₁₀-haloalkoxy, C₁-C₁₀-haloalkylthio, C₁-C₁₀-alkoxycarbonyl, (C₁-C₁₀-alkyl)amino, di-(C₁-C₁₀-alkyl)amino, C₃-C₁₀-cycloalkyl and phenyl, it being possible for phenyl to be unsubstituted, partially or fully halogenated and/or to carry 1, 2 or 3 substituents, independently of one another selected from the group consisting of C₁-C₄-alkyl, C₁-C₄-haloalkyl, C₁-C₄-alkoxy and C₁-C₄-haloalkoxy;
wherein
R^{5a} is C₁-C₁₀-alkyl, C₃-C₁₀-cycloalkyl, C₂-C₁₀-alkenyl, C₂-C₁₀-alkynyl, aryl, aryl-C₁-C₄-alkyl, heteroaryl or heteroaryl-C₁-C₄-alkyl, heterocyclyl or heterocyclyl-C₁-C₄-alkyl;
R^{5b}, R^{5c}, independently from each other, are selected from hydrogen, C₁-C₁₀-alkyl, C₃-C₁₀-cycloalkyl, C₂-C₁₀-alkenyl, C₂-C₁₀-alkynyl, aryl, aryl-C₁-C₄-alkyl, heteroaryl and heteroaryl-C₁-C₄-alkyl, heterocyclyl or heterocyclyl-C₁-C₄-alkyl;
wherein C₁-C₁₀-alkyl, C₃-C₁₀-cycloalkyl, C₂-C₁₀-alkenyl and C₂-C₁₀-alkynyl in R⁵, R^{5b} and R^{5c} may be unsubstituted, partially or fully halogenated and/or may carry 1, 2 or 3 radicals, selected from the group consisting of cyano, nitro, amino, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, (C₁-C₄-alkoxy)carbonyl, (C₁-C₄-alkyl)amino, di(C₁-C₄-alkyl)amino and C₃-C₈-cycloalkyl;
wherein the heteroaryl moiety in heteroaryl and heteroaryl-C₁-C₄-alkyl of R⁵, R^{5a}, R^{5b} and R^{5c} is 5- or 6 membered and contains 1, 2, 3 or 4 heteroatoms and/or heteroatom groups, selected from O, S, SO, SO₂, N or NR^{a}, as ring members, R^{a} being hydrogen or C₁-C₆-alkyl;
wherein the heterocyclyl moiety in heterocyclyl and heterocyclyl-C₁-C₄-alkyl of R⁵, R^{5a}, R^{5b} and R^{5c} is 3- to 7 membered, is saturated or partly unsaturated and contains 1, 2, 3 or 4 heteroatoms and/or heteroatom groups, selected from O, S, SO, SO₂, N or NR^{b}, as ring members, R^{b} being hydrogen or C₁-C₆-alkyl, and additionally may contain 1, 2 or 3 CO groups as ring members;
and wherein the carbon atoms of aryl, hetaryl, and heterocyclyl moieties in R⁵, R^{5a}, R^{5b} and R^{5c} may be unsubstituted or may carry 1, 2 or 3 substituents, independently of one another selected from the group consisting of halogen, C₁-C₄-alkyl, C₁-C₄-haloalkyl, C₁-C₄-alkoxy and C₁-C₄-haloalkoxy;
R⁶ independently has one of the meanings given for R⁵;
R⁷, R⁸, independently from each other, are selected from aryl, aryl-C₁-C₄-alkyl, heteroaryl, heteroaryl-C₁-C₄-alkyl, heterocyclyl, heterocyclyl-C₁-C₄-alkyl, C₁-C₁₀-alkyl, C₃-C₁₀-cycloalkyl, C₂-C₁₀-alkenyl and C₂-C₁₀-alkynyl, wherein the four last-mentioned radicals may be unsubstituted, partially or fully halogenated and/or may carry 1, 2 or 3 radicals, independently of one another each selected from the group consisting of cyano, nitro, amino, C₁-C₁₀-alkoxy, C₁-C₁₀-alkylthio, C₁-C₁₀-alkylsulfinyl, C₁-C₁₀-alkylsulfonyl, C₁-C₁₀-haloalkoxy, C₁-C₁₀-haloalkylthio, C₁-C₁₀-alkoxycarbonyl, (C₁-C₁₀-alkyl)amino, di-(C₁-C₁₀-alkyl)amino, C₃-C₁₀-cycloalkyl and phenyl, it being possible for phenyl to be unsubstituted, partially or fully halogenated and/or to carry 1, 2 or 3 substituents, independently of one another selected from the group consisting of C₁-C₄-alkyl, C₁-C₄-haloalkyl, C₁-C₄-alkoxy and C₁-C₄-haloalkoxy; or
R⁷ and R⁸ together with the sulfur atom they are bound to form a saturated or ethylenically unsaturated 5- to 10-membered ring, optionally substituted by 1, 2, 3 or 4 radicals selected from C₁-C₅-alkyl and halogen, wherein the ring may contain, in addition to the sulfur atom, 1, 2 or 3 heteroatoms and/or heteroatom-containing groups as ring members selected from the group consisting of nitrogen, oxygen, sulfur, CO, SO, SO₂ and N—R¹⁷;
wherein the heteroaryl moiety in heteroaryl and heteroaryl-C₁-C₄-alkyl of R⁷ and R⁸ is 5- or 6 membered and contains 1, 2, 3 or 4 heteroatoms and/or heteroatom groups, selected from O, S, SO, SO₂, N or NR^{c}, as ring members, R^{c} being hydrogen or C₁-C₆-alkyl,
wherein the heterocyclyl moiety in heterocyclyl and heterocyclyl-C₁-C₄-alkyl of R⁷ and R⁸ is 3- to 7-membered, is saturated or partly unsaturated and contains 1, 2, 3 or 4 heteroatoms and/or heteroatom groups, selected from O, S, SO, SO₂, N or NR^{d}, as ring members, R^{d} being hydrogen or C₁-C₆-alkyl, and additionally may contain 1, 2 or 3 CO groups as ring members,
and wherein the carbon atoms of aryl, hetaryl, and heterocyclyl in R⁷ and R⁸ may be unsubstituted or may carry 1, 2 or 3 substituents, independently of one another selected from the group consisting of halogen, C₁-C₄-alkyl, C₁-C₄-haloalkyl, C₁-C₄-alkoxy and C₁-C₄-haloalkoxy;
R⁹ is selected from the group consisting of hydrogen, OR^{9a}, NR^{9b} bR^{9c}, aryl, aryl-C₁-C₄-alkyl, heteroaryl, heteroaryl-C₁-C₄-alkyl, heterocyclyl, heterocyclyl-C₁-C₄-alkyl, C₁-C₁₀-alkyl, C₂-C₁₀-alkenyl, C₂-C₁₀-alkynyl and C₃-C₁₀-cycloalkyl, wherein the four last-mentioned radicals may be unsubstituted, partially or fully halogenated and/or may carry 1, 2 or 3 radicals, independently of one another each selected from the group consisting of cyano, nitro, amino, C₁-C₁₀-alkoxy, C₁-C₁₀-alkylthio, C₁-C₁₀-alkylsulfinyl, C₁-C₁₀-alkylsulfonyl, C₁-C₁₀-haloalkoxy, C₁-C₁₀-haloalkylthio, C₁-C₁₀-alkoxycarbonyl, (C₁-C₁₀-alkyl)amino, di-(C₁-C₁₀ alkyl)amino, C₃-C₁₀-cycloalkyl and phenyl, it being possible for phenyl to be unsubstituted, partially or fully halogenated and/or to carry 1, 2 or 3 substituents, independently of one another selected from the group consisting of C₁-C₄-alkyl, C₁-C₄-haloalkyl, C₁-C₄-alkoxy and C₁-C₄-haloalkoxy,
and wherein
R^{9a} is C₁-C₁₀-alkyl, C₃-C₁₀-cycloalkyl, C₂-C₁₀-alkenyl, C₂-C₁₀-alkynyl, aryl, aryl-C₁-C₄-alkyl, heteroaryl or heteroaryl-C₁-C₄-alkyl, heterocyclyl or heterocyclyl-C₁-C₄-alkyl,
R^{9b}, R^{9c}, independently from each other, are selected from hydrogen, C₁-C₁₀-alkyl, C₃-C₁₀-cycloalkyl, C₂-C₁₀-alkenyl, C₂-C₁₀-alkynyl, aryl, aryl-C₁-C₄-alkyl, heteroaryl and heteroaryl-C₁-C₄-alkyl, heterocyclyl or heterocyclyl-C₁-C₄-alkyl,
wherein C₁-C₁₀-alkyl, C₃-C₁₀-cycloalkyl, C₂-C₁₀-alkenyl and C₂-C₁₀-alkynyl in R^{9a}, R^{9b} and R^{9c} may be unsubstituted, partially or fully halogenated and/or may carry 1, 2 or 3 radicals, selected from the group consisting of cyano, nitro, amino, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, (C₁-C₄-alkoxy)carbonyl, (C₁-C₄-alkyl)amino, di(C₁-C₄-alkyl)amino and C₃-C₈-cycloalkyl,
wherein the heteroaryl moiety in heteroaryl and heteroaryl-C₁-C₄-alkyl of R⁹, R^{9a}, R^{9b} and R^{9c} is 5- or 6-membered and contains 1, 2, 3 or 4 heteroatoms and/or heteroatom groups, selected from O, S, SO, SO₂, N or NR^{e}, as ring members, R^{e} being hydrogen or C₁-C₆-alkyl,
wherein the heterocyclyl moiety in heterocyclyl and heterocyclyl-C₁-C₄-alkyl of R⁹, R^{9a}, R^{9b} and R^{9c} is 3- to 7-membered, is saturated or partly unsaturated and contains 1, 2, 3 or 4 heteroatoms and/or heteroatom groups, selected from O, S, SO, SO₂, N or NR^{f}, as ring members, R^{f} being hydrogen or C₁-C₆-alkyl, and additionally may contain 1, 2 or 3 CO groups as ring members,
and wherein the carbon atoms of aryl, hetaryl and heterocyclyl moieties in R⁹, R^{9a}, R^{9b} and R^{9c} may be unsubstituted or may carry 1, 2 or 3 substituents, independently of one another selected from the group consisting of halogen, C₁-C₄-alkyl, C₁-C₄-haloalkyl, C₁-C₄-alkoxy and C₁-C₄-haloalkoxy;
R¹⁰ is selected from the group consisting of hydrogen, C(═O)—R¹⁶, C₁-C₁₀-alkyl, C₂-C₁₀-alkenyl, C₂-C₁₀-alkynyl, C₁-C₁₀-alkoxy and C₃-C₁₀-cycloalkyl, wherein the five last-mentioned radicals may be unsubstituted, partially or fully halogenated and/or may carry 1, 2 or 3 radicals, independently of one another each selected from the group consisting of cyano, nitro, amino, C₁-C₁₀-alkoxy, C₁-C₁₀-alkylthio, C₁-C₁₀-alkylsulfinyl, C₁-C₁₀-alkylsulfonyl, C₁-C₁₀-haloalkoxy, C₁-C₁₀-haloalkylthio, C₁-C₁₀-alkoxycarbonyl, (C₁-C₁₀-alkyl)amino, di-(C₁-C₁₀-alkyl)amino, C₃-C₁₀-cycloalkyl and phenyl, it being possible for phenyl to be unsubstituted, partially or fully halogenated and/or to carry 1, 2 or 3 substituents, independently of one another selected from the group consisting of C₁-C₄-alkyl, C₁-C₄-haloalkyl, C₁-C₄-alkoxy and C₁-C₄-haloalkoxy; or
R⁹ and R¹⁰ together with the adjacent nitrogen and carbon atoms form a saturated or ethylenically unsaturated 5 to 10-membered ring, optionally substituted by 1, 2, 3 or 4 radicals selected from C₁-C₅-alkyl and halogen, wherein the ring may contain, in addition to the nitrogen and carbon ring members, 1, 2 or 3 heteroatoms and/or heteroatom groups as ring members selected from the group consisting of nitrogen, oxygen, sulfur, CO, SO, SO₂ and N—R¹⁷;
R¹² is hydrogen, C₁-C₆-alkyl, C₁-C₄-alkoxy-C₁-C₄-alkyl or C₁-C₆-alkoxy;
each R¹³ independently is selected from halogen, cyano, nitro, sulfenylamino, sulfinylamino, sulfonylamino, aryl, aryl-C₁-C₄-alkyl, heteroaryl, heteroaryl-C₁-C₄-alkyl, heterocyclyl, heterocyclyl-C₁-C₄-alkyl, C₁-C₆-alkyl, C₃-C₈-cycloalkyl, C₁-C₆-alkoxy, C₁-C₆-alkoxycarbonyl, C₁-C₆-alkylcarbonyl, (C₁-C₆-alkyl)amino, di(C₁-C₆-alkyl)amino, C₁-C₆-alkylthio, C₁-C₆-alkylsulfinyl or C₁-C₆-alkylsulfonyl, wherein the ten last-mentioned radicals may be unsubstituted, partially or fully halogenated and/or may carry 1, 2 or 3 radicals, selected from the group consisting of cyano, nitro, amino, OH, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, C₁-C₄-alkylcarbonyloxy, (C₁-C₄-alkoxy)carbonyl, (C₁-C₄-alkyl)amino, di(C₁-C₄-alkyl)amino, C₃-C₈-cycloalkyl and phenyl, it being possible for phenyl to be unsubstituted, partially or fully halogenated and/or to carry 1, 2 or 3 substituents, independently of one another selected from the group consisting of C₁-C₄-alkyl, C₁-C₄-haloalkyl, C₁-C₄-alkoxy and C₁-C₄-haloalkoxy,
wherein the heteroaryl moiety in heteroaryl and heteroaryl-C₁-C₄-alkyl of R¹³ is 5- or 6 membered and contains 1, 2, 3 or 4 heteroatoms and/or heteroatom groups, selected from O, S, SO, SO₂, N or NR^{g}, as ring members, R^{g} being hydrogen or C₁-C₆-alkyl,
wherein the heterocyclyl moiety in heterocyclyl and heterocyclyl-C₁-C₄-alkyl of R¹³ is 3- to 7-membered and contains 1, 2, 3 or 4 heteroatoms and/or heteroatom groups, selected from O, S, SO, SO₂, N or NR^{h}, as ring members, R^{h} being hydrogen or C₁-C₆-alkyl, and additionally may contain 1, 2 or 3 CO groups as ring members,
and wherein the carbon atoms of aryl, hetaryl, and heterocyclyl in R¹³ may be unsubstituted or may carry 1, 2 or 3 substituents, independently of one another selected from the group consisting of halogen, C₁-C₄-alkyl, C₁-C₄-haloalkyl, C₁-C₄-alkoxy and C₁-C₄-haloalkoxy;
R¹⁴ and R⁵, independently of one another, are selected from the group consisting of hydrogen, hydroxy, C₁-C₆-alkoxy, amino, C₁-C₄-alkylamino, di(C₁-C₄-alkyl)amino, aryl, aryl-C₁-C₄-alkyl, C₁-C₄-alkyl, where the alkyl moiety in the two last-mentioned radicals and the aryl moiety in aryl or aryl-C₁-C₄-alkyl may be partially or fully halogenated,
3- to 7-membered heteroaryl or heteroaryl-C₁-C₄-alkyl, wherein the heteroaryl ring contains as ring members 1, 2 or 3 heteroatoms and/or heteroatom groups, selected from the group consisting of nitrogen, oxygen, sulfur, SO, SO₂ and N—Rⁿ, wherein Rⁿ is hydrogen or C₁-C₄-alkyl,
3- to 7-membered heterocyclyl or heterocyclyl-C₁-C₄-alkyl, wherein the heterocyclic ring is saturated or partly unsaturated and contains 1, 2 or 3 heteroatoms and/or heteroatom groups, selected from the group consisting of nitrogen, oxygen, sulfur, group SO, SO₂ and N—R^{o}, wherein R^{o} is hydrogen or C₁-C₄-alkyl,
and wherein the carbon atoms of the heterocyclic rings may be unsubstituted or substituted by 1 or 2 radicals selected from halogen or C₁-C₄-alkyl;
R¹⁶ is C₁-C₆-alkyl, aryl, aryl-C₁-C₄-alkyl,
5- to 7-membered heteroaryl or heteroaryl-C₁-C₄-alkyl, wherein the heteroaryl ring contains as ring members 1, 2 or 3 heteroatoms and/or heteroatom groups, selected from the group consisting of nitrogen, oxygen, sulfur, SO, SO₂ and N—R^{k}, wherein R^{k} is hydrogen or C₁-C₄-alkyl,
3- to 7-membered heterocyclyl or heterocyclyl-C₁-C₄-alkyl, wherein the heterocyclic ring is saturated or partly unsaturated and contains as ring members 1, 2 or 3 heteroatoms and/or heteroatom groups, selected from the group consisting of nitrogen, oxygen, sulfur, SO, SO₂ and N—R^{m}, wherein R^{m} is hydrogen or C₁-C₄-alkyl,
and wherein the carbon atoms of the heterocyclic rings may be unsubstituted or substituted by 1 or 2 radicals selected from halogen or C₁-C₄-alkyl;
or
R¹⁶ and R⁹ together with the adjacent nitrogen and carbon atoms form a saturated or ethylenically unsaturated 5 to 10-membered ring, where the ring may be partially or fully halogenated and/or may be substituted by 1, 2 or 3 substituents selected from C₁-C₅-alkyl and C₁-C₅-haloalkyl, wherein the ring may contain, in addition to the nitrogen and carbon ring members, 1, 2 or 3 heteroatoms and/or heteroatom groups as ring members selected from the group consisting of nitrogen, oxygen, sulfur, SO, SO₂ and N—R¹⁷;
each R¹⁷ independently is hydrogen, heteroaryl, heteroaryl-C₁-C₄-alkyl or C₁-C₆-alkyl, where the alkyl moiety in the two last-mentioned radicals may be partially or fully halogenated;
and/or the agriculturally acceptable salts thereof.

51. The method of claim 50, wherein the compound of formula I is applied in an amount of from 0.1 g to 10 kg per 100 kg of seeds.

52. The method of claim 50, wherein the resulting plant's shoots are protected from aphids.

53. A seed comprising a compound of the formula I or an agriculturally useful salt thereof in an amount of from 0.1 g to 10 kg per 100 kg of seed: 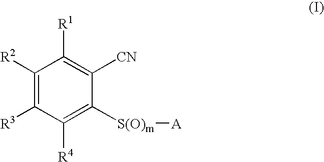
wherein
m is 0, 1 or 2;
A is a radical of the formulae
N═CR⁵R⁶, N═SR⁷R⁸, NR¹⁰—C(═X)—R⁹,
where X is O or S,
or A is a N-bound 5-, 6- or 7-membered heterocycle, which is ethylenically unsaturated or aromatic, and which additionally may contain 1, 2, or 3 further heteroatoms or heteroatom groups, selected from O, S, SO, SO₂, N, and NR¹² and/or 1, 2 or 3 carbonyl groups as ring members and which may carry 1, 2, 3 or 4 radicals R¹³,
R¹ is hydrogen, nitro, cyano, azido, amino, halogen, sulfenylamino, sulfinylamino, sulfonyl amino, C(═O)R¹⁴, C₁-C₆-alkyl, C₂-C₆-alkenyl, C₂-C₆-alkynyl, C₃-C₈-cycloalkyl, C₁-C₆-alkoxy, (C₁-C₆-alkyl)amino, di(C₁-C₆-alkyl)amino, C₁-C₆-alkylthio, C₁-C₆-alkylsulfinyl or C₁-C₆-alkylsulfonyl, wherein the ten last-mentioned radicals may be unsubstituted, partially or fully halogenated and/or may carry 1, 2 or 3 radicals, selected from the group consisting of cyano, nitro, amino, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, (C₁-C₄-alkoxy)carbonyl, (C₁-C₄-alkyl)amino, di(C₁-C₄-alkyl)amino, C₃-C₈-cycloalkyl and phenyl, it being possible for phenyl to be unsubstituted, partially or fully halogenated and/or to carry 1, 2 or 3 substituents, independently of one another selected from the group consisting of C₁-C₄-alkyl, C₁-C₄-haloalkyl, C₁-C₄-alkoxy and C₁-C₄-haloalkoxy;
R², R³ and R⁴ are independently of one another selected from the group consisting of hydrogen, halogen, cyano, azido, nitro, C₁-C₆-alkyl, C₃-C₈-cycloalkyl, C₁-C₄-haloalkyl, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, C₂-C₆-alkenyl, C₂-C₆-alkynyl, amino, (C₁-C₄-alkyl)amino, di(C₁-C₄-alkyl)amino, sulfonylamino, sulfinylamino, sulfenylamino and C(═O)—R¹⁵;
R⁵ is H, OR^{5a}, NR^{5b}R^{5c}, aryl, aryl-C₁-C₄-alkyl, heteroaryl, heteroaryl-C₁-C₄-alkyl, heterocyclyl, heterocyclyl-C₁-C₄-alkyl, C₁-C₁₀-alkyl, C₃-C₁₀-cycloalkyl, C₁-C₁₀-alkylthio, C₁-C₁₀-alkylsulfonyl, C₁-C₁₀-alkylsulfinyl, wherein the five last-mentioned radicals may be unsubstituted, partially or fully halogenated and/or may carry 1, 2 or 3 radicals, independently of one another each selected from the group consisting of cyano, nitro, amino, C₁-C₁₀-alkoxy, C₁-C₁₀-alkylthio, C₁-C₁₀-alkylsulfinyl, C₁-C₁₀-alkylsulfonyl, C₁-C₁₀-haloalkoxy, C₁-C₁₀-haloalkylthio, C₁-C₁₀-alkoxycarbonyl, (C₁-C₁₀-alkyl)amino, di-(C₁-C₁₀-alkyl)amino, C₃-C₁₀-cycloalkyl and phenyl, it being possible for phenyl to be unsubstituted, partially or fully halogenated and/or to carry 1, 2 or 3 substituents, independently of one another selected from the group consisting of C₁-C₄-alkyl, C₁-C₄-haloalkyl, C₁-C₄-alkoxy and C₁-C₄-haloalkoxy;
wherein
R^{5a} is C₁-C₁₀-alkyl, C₃-C₁₀-cycloalkyl, C₂-C₁₀-alkenyl, C₂-C₁₀-alkynyl, aryl, aryl-C₁-C₄-alkyl, heteroaryl or heteroaryl-C₁-C₄-alkyl, heterocyclyl or heterocyclyl-C₁-C₄-alkyl;
R^{5b}, R^{5c}, independently from each other, are selected from hydrogen, C₁-C₁₀-alkyl, C₃-C₁₀-cycloalkyl, C₂-C₁₀-alkenyl, C₂-C₁₀-alkynyl, aryl, aryl-C₁-C₄-alkyl, heteroaryl and heteroaryl-C₁-C₄-alkyl, heterocyclyl or heterocyclyl-C₁-C₄-alkyl;
wherein C₁-C₁₀-alkyl, C₃-C₁₀-cycloalkyl, C₂-C₁₀-alkenyl and C₂-C₁₀-alkynyl in R^{5a}, R^{5b} and R^{5c} may be unsubstituted, partially or fully halogenated and/or may carry 1, 2 or 3 radicals, selected from the group consisting of cyano, nitro, amino, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, (C₁-C₄-alkoxy)carbonyl, (C₁-C₄-alkyl)amino, di(C₁-C₄-alkyl)amino and C₃-C₈-cycloalkyl;
wherein the heteroaryl moiety in heteroaryl and heteroaryl-C₁-C₄-alkyl of R⁵, R^{5a}, R^{5b} and R^{5c} is 5- or 6 membered and contains 1, 2, 3 or 4 heteroatoms and/or heteroatom groups, selected from O, S, SO, SO₂, N or NR^{a}, as ring members, R^{a} being hydrogen or C₁-C₆-alkyl;
wherein the heterocyclyl moiety in heterocyclyl and heterocyclyl-C₁-C₄-alkyl of R⁵, R^{5a}, R^{5b} and R^{5c} is 3- to 7 membered, is saturated or partly unsaturated and contains 1, 2, 3 or 4 heteroatoms and/or heteroatom groups, selected from O, S, SO, SO₂, N or NR^{b}, as ring members, R^{b} being hydrogen or C₁-C₆-alkyl, and additionally may contain 1, 2 or 3 CO groups as ring members;
and wherein the carbon atoms of aryl, hetaryl, and heterocyclyl moieties in R⁵, R^{5a}, R^{5b} and R^{5c} may be unsubstituted or may carry 1, 2 or 3 substituents, independently of one another selected from the group consisting of halogen, C₁-C₄-alkyl, C₁-C₄-haloalkyl, C₁-C₄-alkoxy and C₁-C₄-haloalkoxy;
R⁶ independently has one of the meanings given for R⁵;
R⁷, R⁸, independently from each other, are selected from aryl, aryl-C₁-C₄-alkyl, heteroaryl, heteroaryl-C₁-C₄-alkyl, heterocyclyl, heterocyclyl-C₁-C₄-alkyl, C₁-C₁₀-alkyl, C₃-C₁₀-cycloalkyl, C₂-C₁₀-alkenyl and C₂-C₁₀-alkynyl, wherein the four last-mentioned radicals may be unsubstituted, partially or fully halogenated and/or may carry 1, 2 or 3 radicals, independently of one another each selected from the group consisting of cyano, nitro, amino, C₁-C₁₀-alkoxy, C₁-C₁₀-alkylthio, C₁-C₁₀-alkylsulfinyl, C₁-C₁₀-alkylsulfonyl, C₁-C₁₀-haloalkoxy, C₁-C₁₀-haloalkylthio, C₁-C₁₀-alkoxycarbonyl, (C₁-C₁₀-alkyl)amino, di-(C₁-C₁₀-alkyl)amino, C₃-C₁₀-cycloalkyl and phenyl, it being possible for phenyl to be unsubstituted, partially or fully halogenated and/or to carry 1, 2 or 3 substituents, independently of one another selected from the group consisting of C₁-C₄-alkyl, C₁-C₄-haloalkyl, C₁-C₄-alkoxy and C₁-C₄-haloalkoxy; or
R⁷ and R⁸ together with the sulfur atom they are bound to form a saturated or ethylenically unsaturated 5- to 10-membered ring, optionally substituted by 1, 2, 3 or 4 radicals selected from C₁-C₅-alkyl and halogen, wherein the ring may contain, in addition to the sulfur atom, 1, 2 or 3 heteroatoms and/or heteroatom-containing groups as ring members selected from the group consisting of nitrogen, oxygen, sulfur, CO, SO, SO₂ and N—R¹⁷;
wherein the heteroaryl moiety in heteroaryl and heteroaryl-C₁-C₄-alkyl of R⁷ and R⁸ is 5- or 6 membered and contains 1, 2, 3 or 4 heteroatoms and/or heteroatom groups, selected from O, S, SO, SO₂, N or NR^{c}, as ring members, R^{c} being hydrogen or C₁-C₆-alkyl,
wherein the heterocyclyl moiety in heterocyclyl and heterocyclyl-C₁-C₄-alkyl of R⁷ and R⁸ is 3- to 7-membered, is saturated or partly unsaturated and contains 1, 2, 3 or 4 heteroatoms and/or heteroatom groups, selected from O, S, SO, SO₂, N or NR^{d}, as ring members, R^{d} being hydrogen or C₁-C₆-alkyl, and additionally may contain 1, 2 or 3 CO groups as ring members,
and wherein the carbon atoms of aryl, hetaryl, and heterocyclyl in R⁷ and R⁸ may be unsubstituted or may carry 1, 2 or 3 substituents, independently of one another selected from the group consisting of halogen, C₁-C₄-alkyl, C₁-C₄-haloalkyl, C₁-C₄-alkoxy and C₁-C₄-haloalkoxy;
R⁹ is selected from the group consisting of hydrogen, OR^{9a}, NR^{9b}R^{9c}, aryl, aryl-C₁-C₄-alkyl, heteroaryl, heteroaryl-C₁-C₄-alkyl, heterocyclyl, heterocyclyl-C₁-C₄-alkyl, C₁-C₁₀-alkyl, C₂-C₁₀-alkenyl, C₂-C₁₀-alkynyl and C₃-C₁₀-cycloalkyl, wherein the four last-mentioned radicals may be unsubstituted, partially or fully halogenated and/or may carry 1, 2 or 3 radicals, independently of one another each selected from the group consisting of cyano, nitro, amino, C₁-C₁₀-alkoxy, C₁-C₁₀-alkylthio, C₁-C₁₀-alkylsulfinyl, C₁-C₁₀-alkylsulfonyl, C₁-C₁₀-haloalkoxy, C₁-C₁₀-haloalkylthio, C₁-C₁₀-alkoxycarbonyl, (C₁-C₁₀-alkyl)amino, di-(C₁-C₁₀-alkyl)amino, C₃-C₁₀-cycloalkyl and phenyl, it being possible for phenyl to be unsubstituted, partially or fully halogenated and/or to carry 1, 2 or 3 substituents, independently of one another selected from the group consisting of C₁-C₄-alkyl, C₁-C₄-haloalkyl, C₁-C₄-alkoxy and C₁-C₄-haloalkoxy,
and wherein
R^{9a} is C₁-C₁₀-alkyl, C₃-C₁₀-cycloalkyl, C₂-C₁₀-alkenyl, C₂-C₁₀-alkynyl, aryl, aryl-C₁-C₄-alkyl, heteroaryl or heteroaryl-C₁-C₄-alkyl, heterocyclyl or heterocyclyl-C₁-C₄-alkyl,
R^{9b}, R^{9c}, independently from each other, are selected from hydrogen, C₁-C₁₀-alkyl, C₃-C₁₀-cycloalkyl, C₂-C₁₀-alkenyl, C₂-C₁₀-alkynyl, aryl, aryl-C₁-C₄-alkyl, heteroaryl and heteroaryl-C₁-C₄-alkyl, heterocyclyl or heterocyclyl-C₁-C₄-alkyl,
wherein C₁-C₁₀-alkyl, C₃-C₁₀-cycloalkyl, C₂-C₁₀-alkenyl and C₂-C₁₀-alkynyl in R^{9a}, R^{9b} and R^{9c} may be unsubstituted, partially or fully halogenated and/or may carry 1, 2 or 3 radicals, selected from the group consisting of cyano, nitro, amino, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, (C₁-C₄-alkoxy)carbonyl, (C₁-C₄-alkyl)amino, di(C₁-C₄-alkyl)amino and C₃-C₈-cycloalkyl,
wherein the heteroaryl moiety in heteroaryl and heteroaryl-C₁-C₄-alkyl of R⁹, R^{9a}, R^{9b} and R^{9c} is 5- or 6-membered and contains 1, 2, 3 or 4 heteroatoms and/or heteroatom groups, selected from O, S, SO, SO₂, N or NR^{e}, as ring members, R^{e} being hydrogen or C₁-C₆-alkyl,
wherein the heterocyclyl moiety in heterocyclyl and heterocyclyl-C₁-C₄-alkyl of R⁹, R^{9a}, R^{9b} and R^{9c} is 3- to 7-membered, is saturated or partly unsaturated and contains 1, 2, 3 or 4 heteroatoms and/or heteroatom groups, selected from O, S, SO, SO₂, N or NR^{f}, as ring members, R^{f} being hydrogen or C₁-C₆-alkyl, and additionally may contain 1, 2 or 3 CO groups as ring members,
and wherein the carbon atoms of aryl, hetaryl and heterocyclyl moieties in R⁹, R^{9a}, R^{9b} and R^{9c} may be unsubstituted or may carry 1, 2 or 3 substituents, independently of one another selected from the group consisting of halogen, C₁-C₄-alkyl, C₁-C₄-haloalkyl, C₁-C₄-alkoxy and C₁-C₄-haloalkoxy;
R¹⁰ is selected from the group consisting of hydrogen, C(═O)—R¹⁶, C₁-C₁₀-alkyl, C₂-C₁₀-alkenyl, C₂-C₁₀-alkynyl, C₁-C₁₀-alkoxy and C₃-C₁₀-cycloalkyl, wherein the five last-mentioned radicals may be unsubstituted, partially or fully halogenated and/or may carry 1, 2 or 3 radicals, independently of one another each selected from the group consisting of cyano, nitro, amino, C₁-C₁₀-alkoxy, C₁-C₁₀-alkylthio, C₁-C₁₀-alkylsulfinyl, C₁-C₁₀-alkylsulfonyl, C₁-C₁₀-haloalkoxy, C₁-C₁₀-haloalkylthio, C₁-C₁₀-alkoxycarbonyl, (C₁-C₁₀-alkyl)amino, di-(C₁-C₁₀-alkyl)amino, C₃-C₁₀-cycloalkyl and phenyl, it being possible for phenyl to be unsubstituted, partially or fully halogenated and/or to carry 1, 2 or 3 substituents, independently of one another selected from the group consisting of C₁-C₄-alkyl, C₁-C₄-haloalkyl, C₁-C₄-alkoxy and C₁-C₄-haloalkoxy; or
R⁹ and R¹⁰ together with the adjacent nitrogen and carbon atoms form a saturated or ethylenically unsaturated 5 to 10-membered ring, optionally substituted by 1, 2, 3 or 4 radicals selected from C₁-C₅-alkyl and halogen, wherein the ring may contain, in addition to the nitrogen and carbon ring members, 1, 2 or 3 heteroatoms and/or heteroatom groups as ring members selected from the group consisting of nitrogen, oxygen, sulfur, CO, SO, SO₂ and N—R¹⁷;
R¹² is hydrogen, C₁-C₆-alkyl, C₁-C₄-alkoxy-C₁-C₄-alkyl or C₁-C₆-alkoxy;
each R¹³ independently is selected from halogen, cyano, nitro, sulfenylamino, sulfinylamino, sulfonylamino, aryl, aryl-C₁-C₄-alkyl, heteroaryl, heteroaryl-C₁-C₄-alkyl, heterocyclyl, heterocyclyl-C₁-C₄-alkyl, C₁-C₆-alkyl, C₃-C₈-cycloalkyl, C₁-C₆-alkoxy, C₁-C₆-alkoxycarbonyl, C₁-C₆-alkylcarbonyl, (C₁-C₆-alkyl)amino, di(C₁-C₆-alkyl)amino, C₁-C₆-alkylthio, C₁-C₆-alkylsulfinyl or C₁-C₆-alkylsulfonyl, wherein the ten last-mentioned radicals may be unsubstituted, partially or fully halogenated and/or may carry 1, 2 or 3 radicals, selected from the group consisting of cyano, nitro, amino, OH, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, C₁-C₄-alkylcarbonyloxy, (C₁-C₄-alkoxy)carbonyl, (C₁-C₄-alkyl)amino, di(C₁-C₄-alkyl)amino, C₃-C₈-cycloalkyl and phenyl, it being possible for phenyl to be unsubstituted, partially or fully halogenated and/or to carry 1, 2 or 3 substituents, independently of one another selected from the group consisting of C₁-C₄-alkyl, C₁-C₄-haloalkyl, C₁-C₄-alkoxy and C₁-C₄-haloalkoxy,
wherein the heteroaryl moiety in heteroaryl and heteroaryl-C₁-C₄-alkyl of R¹³ is 5- or 6 membered and contains 1, 2, 3 or 4 heteroatoms and/or heteroatom groups, selected from O, S, SO, SO₂, N or NR^{g}, as ring members, R^{g} being hydrogen or C₁-C₆-alkyl,
wherein the heterocyclyl moiety in heterocyclyl and heterocyclyl-C₁-C₄-alkyl of R¹³ is 3- to 7-membered and contains 1, 2, 3 or 4 heteroatoms and/or heteroatom groups, selected from O, S, SO, SO₂, N or NR^{h}, as ring members, R^{h} being hydrogen or C₁-C₆-alkyl, and additionally may contain 1, 2 or 3 CO groups as ring members,
and wherein the carbon atoms of aryl, hetaryl, and heterocyclyl in R¹³ may be unsubstituted or may carry 1, 2 or 3 substituents, independently of one another selected from the group consisting of halogen, C₁-C₄-alkyl, C₁-C₄-haloalkyl, C₁-C₄-alkoxy and C₁-C₄-haloalkoxy;
R¹⁴ and R¹⁵, independently of one another, are selected from the group consisting of hydrogen, hydroxy, C₁-C₆-alkoxy, amino, C₁-C₄-alkylamino, di(C₁-C₄-alkyl)amino, aryl, aryl-C₁-C₄-alkyl, C₁-C₄-alkyl, where the alkyl moiety in the two last-mentioned radicals and the aryl moiety in aryl or aryl-C₁-C₄-alkyl may be partially or fully halogenated,
3- to 7-membered heteroaryl or heteroaryl-C₁-C₄-alkyl, wherein the heteroaryl ring contains as ring members 1, 2 or 3 heteroatoms and/or heteroatom groups, selected from the group consisting of nitrogen, oxygen, sulfur, SO, SO₂ and N—Rⁿ, wherein Rⁿ is hydrogen or C₁-C₄-alkyl,
3- to 7-membered heterocyclyl or heterocyclyl-C₁-C₄-alkyl, wherein the heterocyclic ring is saturated or partly unsaturated and contains 1, 2 or 3 heteroatoms and/or heteroatom groups, selected from the group consisting of nitrogen, oxygen, sulfur, group SO, SO₂ and N—R^{o}, wherein R^{o} is hydrogen or C₁-C₄-alkyl,
and wherein the carbon atoms of the heterocyclic rings may be unsubstituted or substituted by 1 or 2 radicals selected from halogen or C₁-C₄-alkyl;
R¹⁶ is C₁-C₆-alkyl, aryl, aryl-C₁-C₄-alkyl,
5- to 7-membered heteroaryl or heteroaryl-C₁-C₄-alkyl, wherein the heteroaryl ring contains as ring members 1, 2 or 3 heteroatoms and/or heteroatom groups, selected from the group consisting of nitrogen, oxygen, sulfur, SO, SO₂ and N—R^{k}, wherein R^{k} is hydrogen or C₁-C₄-alkyl,
3- to 7-membered heterocyclyl or heterocyclyl-C₁-C₄-alkyl, wherein the heterocyclic ring is saturated or partly unsaturated and contains as ring members 1, 2 or 3 heteroatoms and/or heteroatom groups, selected from the group consisting of nitrogen, oxygen, sulfur, SO, SO₂ and N—R^{m}, wherein R^{m} is hydrogen or C₁-C₄-alkyl,
and wherein the carbon atoms of the heterocyclic rings may be unsubstituted or substituted by 1 or 2 radicals selected from halogen or C₁-C₄-alkyl;
or
R¹⁶ and R⁹ together with the adjacent nitrogen and carbon atoms form a saturated or ethylenically unsaturated 5 to 10-membered ring, where the ring may be partially or fully halogenated and/or may be substituted by 1, 2 or 3 substituents selected from C₁-C₅-alkyl and C₁-C₅-haloalkyl, wherein the ring may contain, in addition to the nitrogen and carbon ring members, 1, 2 or 3 heteroatoms and/or heteroatom groups as ring members selected from the group consisting of nitrogen, oxygen, sulfur, SO, SO₂ and N—R¹⁷;
each R¹⁷ independently is hydrogen, heteroaryl, heteroaryl-C₁-C₄-alkyl or C₁-C₆-alkyl, where the alkyl moiety in the two last-mentioned radicals may be partially or fully halogenated;
and/or the agriculturally acceptable salts thereof.

54. Cyanobenzene compounds of the formula (I) 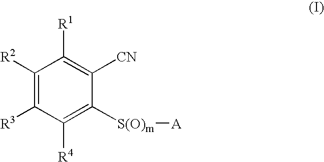
wherein
m is 0, 1 or 2;
A is a radical of the formulae
N═CR⁵R⁶, N═SR⁷R⁸, NR¹⁰—C(═X)—R⁹,
where X is O or S,
or A is a N-bound 5-, 6- or 7-membered heterocycle, which is ethylenically unsaturated or aromatic, and which additionally may contain 1, 2, or 3 further heteroatoms or heteroatom groups, selected from O, S, SO, SO₂, N, and NR¹², and/or 1, 2 or 3 carbonyl groups as ring members and which may carry 1, 2, 3 or 4 radicals R¹³,
R¹ is hydrogen, nitro, cyano, azido, amino, halogen, sulfenylamino, sulfinylamino, sulfonylamino, C(═O)R¹⁴, C₁-C₆-alkyl, C₂-C₆-alkenyl, C₂-C₆-alkynyl, C₃-C₈-cycloalkyl, C₁-C₆-alkoxy, (C₁-C₆-alkyl)amino, di(C₁-C₆-alkyl)amino, C₁-C₆-alkylthio, C₁-C₆-alkylsulfinyl or C₁-C₆-alkylsulfonyl, wherein the ten last-mentioned radicals may be unsubstituted, partially or fully halogenated and/or may carry 1, 2 or 3 radicals, selected from the group consisting of cyano, nitro, amino, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, (C₁-C₄-alkoxy)carbonyl, (C₁-C₄-alkyl)amino, di(C₁-C₄-alkyl)amino, C₃-C₈-cycloalkyl and phenyl, it being possible for phenyl to be unsubstituted, partially or fully halogenated and/or to carry 1, 2 or 3 substituents, independently of one another selected from the group consisting of C₁-C₄-alkyl, C₁-C₄-haloalkyl, C₁-C₄-alkoxy and C₁-C₄-haloalkoxy;
R², R³ and R⁴ are independently of one another selected from the group consisting of hydrogen, halogen, cyano, azido, nitro, C₁-C₆-alkyl, C₃-C₈-cycloalkyl, C₁-C₄-haloalkyl, C₁-C₄-alkoxy, C₁-C₄-alkylthio, C₁-C₄-alkylsulfinyl, C₁-C₄-alkylsulfonyl, C₁-C₄-haloalkoxy, C₁-C₄-haloalkylthio, C₂-C₆-alkenyl, C₂-C₆-alkynyl, amino, (C₁-C₄-alkyl)amino, di(C₁-C₄-alkyl)amino, sulfonylamino, sulfinylamino, sulfenylamino and C(═O)—R¹⁵;
R¹⁶ is C₁-C₆-alkyl, aryl, aryl-C₁-C₄-alkyl,
5- to 7-membered heteroaryl or heteroaryl-C₁-C₄-alkyl, wherein the heteroaryl ring contains as ring members 1, 2 or 3 heteroatoms and/or heteroatom groups, selected from the group consisting of nitrogen, oxygen, sulfur, SO, SO₂ and N—R^{k}, wherein R^{k} is hydrogen or C₁-C₄-alkyl,
3- to 7-membered heterocyclyl or heterocyclyl-C₁-C₄-alkyl, wherein the heterocyclic ring is saturated or partly unsaturated and contains as ring members 1, 2 or 3 heteroatoms and/or heteroatom groups, selected from the group consisting of nitrogen, oxygen, sulfur, SO, SO₂ and N—R^{m}, wherein R^{m} is hydrogen or C₁-C₄-alkyl,
and wherein the carbon atoms of the heterocyclic rings may be unsubstituted or substituted by 1 or 2 radicals selected from halogen or C₁-C₄-alkyl;
or
R¹⁶ and R⁹ together with the adjacent nitrogen and carbon atoms form a saturated or ethylenically unsaturated 5 or 6 membered ring, where the ring may be partially or fully halogenated and/or may be substituted by 1, 2 or 3 substituents selected from C₁-C₅-alkyl and C₁-C₅-haloalkyl, wherein the ring may contain, in addition to the nitrogen and carbon ring members, 1, 2 or 3 heteroatoms and/or heteroatom groups as ring members selected from the group consisting of nitrogen, oxygen, sulfur, SO, SO₂ and N—R¹⁷;
and agriculturally acceptable salts thereof,
except for compounds wherein R¹ is H, NO₂ and NH₂ if R² is H, R³ is H, C₁ or CO₂CH₃, R⁴ is H, and A is a radical N═CH—N(CH₃)₂ and also
except for compounds wherein R¹ is H, R² is H or Cl, R³ is H, R⁴ is H and A is an optionally substituted pyridazin-6-on-1-yl-radical or an optionally substituted imidazolin-5-on-1-yl radical, and also
except for compounds wherein R¹ is H, R² is H, R³ is H, R⁴ is H and A is a radical N═C(O-ethyl)₂. and also
except for compounds wherein A is a radical of the formula 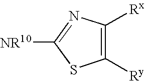
wherein R^{x} and R^{y} are, independently of each other, hydrogen or C₁-C₅-alkyl and R¹⁰ is H or C₁-C₁₀-alkyl.

55. An agricultural composition comprising at least one compound of claim 54 and at least one inert liquid and/or solid agriculturally acceptable carrier.
